SEQUENCE LISTING (322 sequences)

SEQ ID NO: 1 - DNA, 33 bp, Artificial Sequence
  Description of Artificial Sequence Synthetic oligonucleotide
  Description of Combined DNA/RNA Molecule Synthetic oligonucleotide
  modified_base (1)..(10): a, c, t, g, unknown or other
  misc_feature (1)..(10): This region may encompass 2-10, 3-8, or 4-7 non-uracil nucleobases, wherein some positions may be absent
  modified_base (12)..(21): a, c, t, g, unknown or other
  misc_feature (12)..(21): This region may encompass 2-10, 3-8, or 4-7 non-uracil nucleobases, wherein some positions may be absent
  modified_base (23)..(32): a, c, t, g, unknown or other
  misc_feature (23)..(32): This region may encompass 2-10, 3-8, or 4-7 non-uracil nucleobases, wherein some positions may be absent
  See specification as filed for detailed description of substitutions and preferred embodiments
nnnnnnnnnnunnnnnnnnnnunnnnnnnnnnu

SEQ ID NO: 2 - DNA, 33 bp, Artificial Sequence; identical to SEQ ID NO: 1
  Description of Artificial Sequence Synthetic oligonucleotide
  Description of Combined DNA/RNA Molecule Synthetic oligonucleotide
  modified_base (1)..(10): a, c, t, g, unknown or other
  misc_feature (1)..(10): This region may encompass 2-10, 3-8, or 4-7 non-uracil nucleobases, wherein some positions may be absent
  modified_base (12)..(21): a, c, t, g, unknown or other
  misc_feature (12)..(21): This region may encompass 2-10, 3-8, or 4-7 non-uracil nucleobases, wherein some positions may be absent
  modified_base (23)..(32): a, c, t, g, unknown or other
  misc_feature (23)..(32): This region may encompass 2-10, 3-8, or 4-7 non-uracil nucleobases, wherein some positions may be absent
  See specification as filed for detailed description of substitutions and preferred embodiments

SEQ ID NO: 3 - DNA, 44 bp, Artificial Sequence
  Description of Artificial Sequence Synthetic oligonucleotide
  Description of Combined DNA/RNA Molecule Synthetic oligonucleotide
  modified_base (1)..(10): a, c, t, g, unknown or other
  misc_feature (1)..(10): This region may encompass 2-10, 3-8, or 4-7 non-uracil nucleobases, wherein some positions may be absent
  modified_base (12)..(21): a, c, t, g, unknown or other
  misc_feature (12)..(21): This region may encompass 2-10, 3-8, or 4-7 non-uracil nucleobases, wherein some positions may be absent
  modified_base (23)..(32): a, c, t, g, unknown or other
  misc_feature (23)..(32): This region may encompass 2-10, 3-8, or 4-7 non-uracil nucleobases, wherein some positions may be absent
  modified_base (34)..(43): a, c, t, g, unknown or other
  misc_feature (34)..(43): This region may encompass 2-10, 3-8, or 4-7 non-uracil nucleobases, wherein some positions may be absent
  See specification as filed for detailed description of substitutions and preferred embodiments
nnnnnnnnnnunnnnnnnnnnunnnnnnnnnnunnnnnnnnnnu

SEQ ID NO: 4 - DNA, 44 bp, Artificial Sequence; identical to SEQ ID NO: 3
  Description of Artificial Sequence Synthetic oligonucleotide
  Description of Combined DNA/RNA Molecule Synthetic oligonucleotide
  modified_base (1)..(10): a, c, t, g, unknown or other
  misc_feature (1)..(10): This region may encompass 2-10, 3-8, or 4-7 non-uracil nucleobases, wherein some positions may be absent
  modified_base (12)..(21): a, c, t, g, unknown or other
  misc_feature (12)..(21): This region may encompass 2-10, 3-8, or 4-7 non-uracil nucleobases, wherein some positions may be absent
  modified_base (23)..(32): a, c, t, g, unknown or other
  misc_feature (23)..(32): This region may encompass 2-10, 3-8, or 4-7 non-uracil nucleobases, wherein some positions may be absent
  modified_base (34)..(43): a, c, t, g, unknown or other
  misc_feature (34)..(43): This region may encompass 2-10, 3-8, or 4-7 non-uracil nucleobases, wherein some positions may be absent
  See specification as filed for detailed description of substitutions and preferred embodiments

SEQ ID NO: 5 - DNA, 20 bp, Artificial Sequence
  Description of Artificial Sequence Synthetic oligonucleotide
gccgtactctcaactcacat

SEQ ID NO: 6 - DNA, 20 bp, Artificial Sequence
  Description of Artificial Sequence Synthetic oligonucleotide
ggacttgtatgctgactgct

SEQ ID NO: 7 - DNA, 20 bp, Artificial Sequence
  Description of Artificial Sequence Synthetic oligonucleotide
gactcgtcagcatcaagact

SEQ ID NO: 8 - DNA, 20 bp, Artificial Sequence
  Description of Artificial Sequence Synthetic oligonucleotide
gttctatccaactgcggtct

SEQ ID NO: 9 - DNA, 20 bp, Artificial Sequence
  Description of Artificial Sequence Synthetic oligonucleotide
gttgggtgggtggtgtgtgt

SEQ ID NO: 10 - DNA, 140 bp, Artificial Sequence
  Description of Artificial Sequence Synthetic polynucleotide
atctgaagcggagtccacacaacaagatcgggttaattgaatgctcagatggtgcgccgtctgtcctctctcttgtcctctacggatcagtagccagagattgttgctactaggctccgagaagagactcgaagcaggat

SEQ ID NO: 11 - DNA, 20 bp, Artificial Sequence
  Description of Artificial Sequence Synthetic oligonucleotide
agaccaggctacgatacaac

SEQ ID NO: 12 - DNA, 20 bp, Artificial Sequence
  Description of Artificial Sequence Synthetic oligonucleotide
agaccgtagggcaatgattc

SEQ ID NO: 13 - DNA, 20 bp, Artificial Sequence
  Description of Artificial Sequence Synthetic oligonucleotide
atgcttgatgagtgacctgg

SEQ ID NO: 14 - DNA, 20 bp, Artificial Sequence
  Description of Artificial Sequence Synthetic oligonucleotide
agcaggtagtgatacttggc

SEQ ID NO: 15 - DNA, 20 bp, Artificial Sequence
  Description of Artificial Sequence Synthetic oligonucleotide
accacacaccacccaccaca

SEQ ID NO: 16 - DNA, 180 bp, Artificial Sequence
  Description of Artificial Sequence Synthetic polynucleotide
gccgtactctcaactcacatatctgaagcggagtccacacaacaagatcgggttaattgaatgctcagatggtgcgccgtctgtcctctctcttgtcctctacggatcagtagccagagattgttgctactaggctccgagaagagactcgaagcaggatagaccaggctacgatacaac

SEQ ID NO: 17 - DNA, 180 bp, Artificial Sequence
  Description of Artificial Sequence Synthetic polynucleotide
ggacttgtatgctgactgctatctgaagcggagtccacacaacaagatcgggttaattgaatgctcagatggtgcgccgtctgtcctctctcttgtcctctacggatcagtagccagagattgttgctactaggctccgagaagagactcgaagcaggatagaccgtagggcaatgattc

SEQ ID NO: 18 - DNA, 180 bp, Artificial Sequence
  Description of Artificial Sequence Synthetic polynucleotide
gactcgtcagcatcaagactatctgaagcggagtccacacaacaagatcgggttaattgaatgctcagatggtgcgccgtctgtcctctctcttgtcctctacggatcagtagccagagattgttgctactaggctccgagaagagactcgaagcaggatatgcttgatgagtgacctgg

SEQ ID NO: 19 - DNA, 180 bp, Artificial Sequence
  Description of Artificial Sequence Synthetic polynucleotide
gttctatccaactgcggtctatctgaagcggagtccacacaacaagatcgggttaattgaatgctcagatggtgcgccgtctgtcctctctcttgtcctctacggatcagtagccagagattgttgctactaggctccgagaagagactcgaagcaggatagcaggtagtgatacttggc

SEQ ID NO: 20 - DNA, 180 bp, Artificial Sequence
  Description of Artificial Sequence Synthetic polynucleotide
gttgggtgggtggtgtgtgtatctgaagcggagtccacacaacaagatcgggttaattgaatgctcagatggtgcgccgtctgtcctctctcttgtcctctacggatcagtagccagagattgttgctactaggctccgagaagagactcgaagcaggataccacacaccacccaccaca

SEQ ID NO: 21 - DNA, 20 bp, Artificial Sequence
  Description of Artificial Sequence Synthetic oligonucleotide
  Description of Combined DNA/RNA Molecule Synthetic oligonucleotide
gccgtactctcaactcacau

SEQ ID NO: 22 - DNA, 20 bp, Artificial Sequence
  Description of Artificial Sequence Synthetic oligonucleotide
  Description of Combined DNA/RNA Molecule Synthetic oligonucleotide
gccgtactcucaactcacau

SEQ ID NO: 23 - DNA, 20 bp, Artificial Sequence
  Description of Artificial Sequence Synthetic oligonucleotide
  Description of Combined DNA/RNA Molecule Synthetic oligonucleotide
gccguactcucaacucacau

SEQ ID NO: 24 - DNA, 20 bp, Artificial Sequence
  Description of Artificial Sequence Synthetic oligonucleotide
gttgtatcgtagcctggtct

SEQ ID NO: 25 - DNA, 20 bp, Artificial Sequence
  Description of Artificial Sequence Synthetic oligonucleotide
  Description of Combined DNA/RNA Molecule Synthetic oligonucleotide
gttgtatcgtagcctggtcu

SEQ ID NO: 26 - DNA, 20 bp, Artificial Sequence
  Description of Artificial Sequence Synthetic oligonucleotide
  Description of Combined DNA/RNA Molecule Synthetic oligonucleotide
gttgtatcguagcctggtcu

SEQ ID NO: 27 - DNA, 20 bp, Artificial Sequence
  Description of Artificial Sequence Synthetic oligonucleotide
  Description of Combined DNA/RNA Molecule Synthetic oligonucleotide
gttguatcguagccuggtcu

SEQ ID NO: 28 - DNA, 20 bp, Artificial Sequence
  Description of Artificial Sequence Synthetic oligonucleotide
  Description of Combined DNA/RNA Molecule Synthetic oligonucleotide
ggacttgtatgctgactgcu

SEQ ID NO: 29 - DNA, 20 bp, Artificial Sequence
  Description of Artificial Sequence Synthetic oligonucleotide
  Description of Combined DNA/RNA Molecule Synthetic oligonucleotide
ggacttgtaugctgactgcu

SEQ ID NO: 30 - DNA, 20 bp, Artificial Sequence
  Description of Artificial Sequence Synthetic oligonucleotide
  Description of Combined DNA/RNA Molecule Synthetic oligonucleotide
ggactugtatgcugactgcu

SEQ ID NO: 31 - DNA, 20 bp, Artificial Sequence
  Description of Artificial Sequence Synthetic oligonucleotide
  Description of Combined DNA/RNA Molecule Synthetic oligonucleotide
ggacutgtaugcugacugcu

SEQ ID NO: 32 - DNA, 20 bp, Artificial Sequence
  Description of Artificial Sequence Synthetic oligonucleotide
gaatcattgccctacggtct

SEQ ID NO: 33 - DNA, 20 bp, Artificial Sequence
  Description of Artificial Sequence Synthetic oligonucleotide
  Description of Combined DNA/RNA Molecule Synthetic oligonucleotide
gaatcattgccctacggtcu

SEQ ID NO: 34 - DNA, 20 bp, Artificial Sequence
  Description of Artificial Sequence Synthetic oligonucleotide
  Description of Combined DNA/RNA Molecule Synthetic oligonucleotide
gaatcautgcccuacggtcu

SEQ ID NO: 35 - DNA, 20 bp, Artificial Sequence
  Description of Artificial Sequence Synthetic oligonucleotide
  Description of Combined DNA/RNA Molecule Synthetic oligonucleotide
gaaucatugcccuacggucu

SEQ ID NO: 36 - DNA, 20 bp, Artificial Sequence
  Description of Artificial Sequence Synthetic oligonucleotide
  Description of Combined DNA/RNA Molecule Synthetic oligonucleotide
gactcgtcagcatcaagacu

SEQ ID NO: 37 - DNA, 20 bp, Artificial Sequence
  Description of Artificial Sequence Synthetic oligonucleotide
  Description of Combined DNA/RNA Molecule Synthetic oligonucleotide
gactcgucagcaucaagacu

SEQ ID NO: 38 - DNA, 20 bp, Artificial Sequence
  Description of Artificial Sequence Synthetic oligonucleotide
ccaggtcactcatcaagcat

SEQ ID NO: 39 - DNA, 20 bp, Artificial Sequence
  Description of Artificial Sequence Synthetic oligonucleotide
  Description of Combined DNA/RNA Molecule Synthetic oligonucleotide
ccaggtcactcatcaagcau

SEQ ID NO: 40 - DNA, 20 bp, Artificial Sequence
  Description of Artificial Sequence Synthetic oligonucleotide
  Description of Combined DNA/RNA Molecule Synthetic oligonucleotide
ccaggucactcaucaagcau

SEQ ID NO: 41 - DNA, 20 bp, Artificial Sequence
  Description of Artificial Sequence Synthetic oligonucleotide
  Description of Combined DNA/RNA Molecule Synthetic oligonucleotide
gttctatccaactgcggtcu

SEQ ID NO: 42 - DNA, 20 bp, Artificial Sequence
  Description of Artificial Sequence Synthetic oligonucleotide
  Description of Combined DNA/RNA Molecule Synthetic oligonucleotide
gttctauccaacugcggtcu

SEQ ID NO: 43 - DNA, 20 bp, Artificial Sequence
  Description of Artificial Sequence Synthetic oligonucleotide
gccaagtatcactacctgct

SEQ ID NO: 44 - DNA, 20 bp, Artificial Sequence
  Description of Artificial Sequence Synthetic oligonucleotide
  Description of Combined DNA/RNA Molecule Synthetic oligonucleotide
gccaagtatcactacctgcu

SEQ ID NO: 45 - DNA, 20 bp, Artificial Sequence
  Description of Artificial Sequence Synthetic oligonucleotide
  Description of Combined DNA/RNA Molecule Synthetic oligonucleotide
gccaaguatcacuacctgcu

SEQ ID NO: 46 - DNA, 20 bp, Artificial Sequence
  Description of Artificial Sequence Synthetic oligonucleotide
tgtggtgggtggtgtgtggt

SEQ ID NO: 47 - DNA, 20 bp, Artificial Sequence
  Description of Artificial Sequence Synthetic oligonucleotide
  Description of Combined DNA/RNA Molecule Synthetic oligonucleotide
tgtggtgggtggtgtgtggu

SEQ ID NO: 48 - DNA, 20 bp, Artificial Sequence
  Description of Artificial Sequence Synthetic oligonucleotide
  Description of Combined DNA/RNA Molecule Synthetic oligonucleotide
tgtggtggguggtgtgtggu

SEQ ID NO: 49 - DNA, 20 bp, Artificial Sequence
  Description of Artificial Sequence Synthetic oligonucleotide
  Description of Combined DNA/RNA Molecule Synthetic oligonucleotide
tgtggugggtggugtgtggu

SEQ ID NO: 50 - DNA, 20 bp, Artificial Sequence
  Description of Artificial Sequence Synthetic oligonucleotide
  Description of Combined DNA/RNA Molecule Synthetic oligonucleotide
tgtgguggguggtgugtggu

SEQ ID NO: 51 - DNA, 20 bp, Artificial Sequence
  Description of Artificial Sequence Synthetic oligonucleotide
  Description of Combined DNA/RNA Molecule Synthetic oligonucleotide
tgugguggguggugtguggu

SEQ ID NO: 52 - DNA, 20 bp, Artificial Sequence
  Description of Artificial Sequence Synthetic oligonucleotide
  Description of Combined DNA/RNA Molecule Synthetic oligonucleotide
gttgggtgggtggtgtgtgu

SEQ ID NO: 53 - DNA, 20 bp, Artificial Sequence
  Description of Artificial Sequence Synthetic oligonucleotide
  Description of Combined DNA/RNA Molecule Synthetic oligonucleotide
gttgggtggguggtgtgtgu

SEQ ID NO: 54 - DNA, 20 bp, Artificial Sequence
  Description of Artificial Sequence Synthetic oligonucleotide
  Description of Combined DNA/RNA Molecule Synthetic oligonucleotide
gttgggugggtggugtgtgu

SEQ ID NO: 55 - DNA, 20 bp, Artificial Sequence
  Description of Artificial Sequence Synthetic oligonucleotide
  Description of Combined DNA/RNA Molecule Synthetic oligonucleotide
gtuggguggguggtgugtgu

SEQ ID NO: 56 - DNA, 340 bp, Artificial Sequence
  Description of Artificial Sequence Synthetic polynucleotide
atctgaagcggagtccacacaacaagatcgggttaattgaatgctcagatggtgcgccgtctgtcctctctcttgtcctctccactatctccactacgccttaggaccagcggtgagctatgtctgaggtcacctacggacccggtttatgtgagcaaccagaattcgccttgactcgtcagcacgttattaaagctattgctatgtttgcggcggacacttatcctggtgtcgtactttcctcgtgaaatcgcgttctgagctggatccctctgagccttacggatcagtagccagagattgttgctactaggctccgagaagagactcgaagcaggat

SEQ ID NO: 57 - DNA, 700 bp, Artificial Sequence
  Description of Artificial Sequence Synthetic polynucleotide
atctgaagcggagtccacacaacaagatcgggttaattgaatgctcagatggtgcgccgtctgtcctctctcttgtcctctccactatctccactacgccttaggaccagcggtgagctatgtctgaggtcacctacggacccggtttatgtgagcaaccagaattcgccttgactcgtcagcacgttattaaagctattgctatgtttgcggcggacacttatcctggtgtcgtactttcctcgtgaaatcgcgttctgcgttgtcttgggtagctatatggactagtgtatacgatcctttgaggagctagaggcggtcactgaactcctcttaatcctgacacaatatggaggtggctggctcgaatcgaccaacctgcgccgcctgagccgacctacggactactcgtgcagtgagctctttatttattagttcgcgctacctttgatacagggcaaacatactcaagctttgtattcactcgccgtcccttcaacgccgcctatgttaacgagattaatgcaaccttaggtccgcagcacacgcataagggattggtgtatgtgggcgcccaaccacaatgcgtgcgcagttagaaagcaattaagtgggactggagctggatccctctgagccttacggatcagtagccagagattgttgctactaggctccgagaagagactcgaagcaggat

SEQ ID NO: 58 - DNA, 38 bp, Artificial Sequence
  Description of Artificial Sequence Synthetic oligonucleotide
gccgtactctcaactcacatatctgaagcggagtccac

SEQ ID NO: 59 - DNA, 41 bp, Artificial Sequence
  Description of Artificial Sequence Synthetic oligonucleotide
gttgtatcgtagcctggtctatcctgcttcgagtctcttct

SEQ ID NO: 60 - DNA, 38 bp, Artificial Sequence
  Description of Artificial Sequence Synthetic oligonucleotide
ggacttgctatgctacgtgtatctgaagcggagtccac

SEQ ID NO: 61 - DNA, 41 bp, Artificial Sequence
  Description of Artificial Sequence Synthetic oligonucleotide
gaatcattgccctacggtctatcctgcttcgagtctcttct

SEQ ID NO: 62 - DNA, 380 bp, Artificial Sequence
  Description of Artificial Sequence Synthetic polynucleotide
gccgtactctcaactcacatatctgaagcggagtccacacaacaagatcgggttaattgaatgctcagatggtgcgccgtctgtcctctctcttgtcctctccactatctccactacgccttaggaccagcggtgagctatgtctgaggtcacctacggacccggtttatgtgagcaaccagaattcgccttgactcgtcagcacgttattaaagctattgctatgtttgcggcggacacttatcctggtgtcgtactttcctcgtgaaatcgcgttctgagctggatccctctgagccttacggatcagtagccagagattgttgctactaggctccgagaagagactcgaagcaggatagaccaggctacgatacaac

SEQ ID NO: 63 - DNA, 380 bp, Artificial Sequence
  Description of Artificial Sequence Synthetic polynucleotide
ggacttgctatgctacgtgtatctgaagcggagtccacacaacaagatcgggttaattgaatgctcagatggtgcgccgtctgtcctctctcttgtcctctccactatctccactacgccttaggaccagcggtgagctatgtctgaggtcacctacggacccggtttatgtgagcaaccagaattcgccttgactcgtcagcacgttattaaagctattgctatgtttgcggcggacacttatcctggtgtcgtactttcctcgtgaaatcgcgttctgagctggatccctctgagccttacggatcagtagccagagattgttgctactaggctccgagaagagactcgaagcaggatagaccgtagggcaatgattc

SEQ ID NO: 64 - DNA, 740 bp, Artificial Sequence
  Description of Artificial Sequence Synthetic polynucleotide
gccgtactctcaactcacatatctgaagcggagtccacacaacaagatcgggttaattgaatgctcagatggtgcgccgtctgtcctctctcttgtcctctccactatctccactacgccttaggaccagcggtgagctatgtctgaggtcacctacggacccggtttatgtgagcaaccagaattcgccttgactcgtcagcacgttattaaagctattgctatgtttgcggcggacacttatcctggtgtcgtactttcctcgtgaaatcgcgttctgcgttgtcttgggtagctatatggactagtgtatacgatcctttgaggagctagaggcggtcactgaactcctcttaatcctgacacaatatggaggtggctggctcgaatcgaccaacctgcgccgcctgagccgacctacggactactcgtgcagtgagctctttatttattagttcgcgctacctttgatacagggcaaacatactcaagctttgtattcactcgccgtcccttcaacgccgcctatgttaacgagattaatgcaaccttaggtccgcagcacacgcataagggattggtgtatgtgggcgcccaaccacaatgcgtgcgcagttagaaagcaattaagtgggactggagctggatccctctgagccttacggatcagtagccagagattgttgctactaggctccgagaagagactcgaagcaggatagaccaggctacgatacaac

SEQ ID NO: 65 - DNA, 740 bp, Artificial Sequence
  Description of Artificial Sequence Synthetic polynucleotide
ggacttgctatgctacgtgtatctgaagcggagtccacacaacaagatcgggttaattgaatgctcagatggtgcgccgtctgtcctctctcttgtcctctccactatctccactacgccttaggaccagcggtgagctatgtctgaggtcacctacggacccggtttatgtgagcaaccagaattcgccttgactcgtcagcacgttattaaagctattgctatgtttgcggcggacacttatcctggtgtcgtactttcctcgtgaaatcgcgttctgcgttgtcttgggtagctatatggactagtgtatacgatcctttgaggagctagaggcggtcactgaactcctcttaatcctgacacaatatggaggtggctggctcgaatcgaccaacctgcgccgcctgagccgacctacggactactcgtgcagtgagctctttatttattagttcgcgctacctttgatacagggcaaacatactcaagctttgtattcactcgccgtcccttcaacgccgcctatgttaacgagattaatgcaaccttaggtccgcagcacacgcataagggattggtgtatgtgggcgcccaaccacaatgcgtgcgcagttagaaagcaattaagtgggactggagctggatccctctgagccttacggatcagtagccagagattgttgctactaggctccgagaagagactcgaagcaggatagaccgtagggcaatgattc

SEQ ID NO: 66 - DNA, 20 bp, Artificial Sequence
  Description of Artificial Sequence Synthetic oligonucleotide
  Description of Combined DNA/RNA Molecule Synthetic oligonucleotide
gccguactcucaacucacau

SEQ ID NO: 67 - DNA, 20 bp, Artificial Sequence
  Description of Artificial Sequence Synthetic oligonucleotide
  Description of Combined DNA/RNA Molecule Synthetic oligonucleotide
gttguatcguagccuggtcu

SEQ ID NO: 68 - DNA, 20 bp, Artificial Sequence
  Description of Artificial Sequence Synthetic oligonucleotide
  Description of Combined DNA/RNA Molecule Synthetic oligonucleotide
ggacutgcuatgcuacgugu

SEQ ID NO: 69 - DNA, 20 bp, Artificial Sequence
  Description of Artificial Sequence Synthetic oligonucleotide
  Description of Combined DNA/RNA Molecule Synthetic oligonucleotide
gaaucatugcccuacggucu

SEQ ID NO: 70 - DNA, 380 bp, Artificial Sequence
  Description of Artificial Sequence Synthetic oligonucleotide
  Description of Combined DNA/RNA Molecule Synthetic oligonucleotide
gccguactcucaacucacauatctgaagcggagtccacacaacaagatcgggttaattgaatgctcagatggtgcgccgtctgtcctctctcttgtcctctccactatctccactacgccttaggaccagcggtgagctatgtctgaggtcacctacggacccggtttatgtgagcaaccagaattcgccttgactcgtcagcacgttattaaagctattgctatgtttgcggcggacacttatcctggtgtcgtactttcctcgtgaaatcgcgttctgagctggatccctctgagccttacggatcagtagccagagattgttgctactaggctccgagaagagactcgaagcaggatagaccaggctacgatacaac

SEQ ID NO: 71 - DNA, 380 bp, Artificial Sequence
  Description of Artificial Sequence Synthetic oligonucleotide
  Description of Combined DNA/RNA Molecule Synthetic oligonucleotide
ggacutgcuatgcuacguguatctgaagcggagtccacacaacaagatcgggttaattgaatgctcagatggtgcgccgtctgtcctctctcttgtcctctccactatctccactacgccttaggaccagcggtgagctatgtctgaggtcacctacggacccggtttatgtgagcaaccagaattcgccttgactcgtcagcacgttattaaagctattgctatgtttgcggcggacacttatcctggtgtcgtactttcctcgtgaaatcgcgttctgagctggatccctctgagccttacggatcagtagccagagattgttgctactaggctccgagaagagactcgaagcaggatagaccgtagggcaatgattc

SEQ ID NO: 72 - DNA, 740 bp, Artificial Sequence
  Description of Artificial Sequence Synthetic oligonucleotide
  Description of Combined DNA/RNA Molecule Synthetic oligonucleotide
gccguactcucaacucacauatctgaagcggagtccacacaacaagatcgggttaattgaatgctcagatggtgcgccgtctgtcctctctcttgtcctctccactatctccactacgccttaggaccagcggtgagctatgtctgaggtcacctacggacccggtttatgtgagcaaccagaattcgccttgactcgtcagcacgttattaaagctattgctatgtttgcggcggacacttatcctggtgtcgtactttcctcgtgaaatcgcgttctgcgttgtcttgggtagctatatggactagtgtatacgatcctttgaggagctagaggcggtcactgaactcctcttaatcctgacacaatatggaggtggctggctcgaatcgaccaacctgcgccgcctgagccgacctacggactactcgtgcagtgagctctttatttattagttcgcgctacctttgatacagggcaaacatactcaagctttgtattcactcgccgtcccttcaacgccgcctatgttaacgagattaatgcaaccttaggtccgcagcacacgcataagggattggtgtatgtgggcgcccaaccacaatgcgtgcgcagttagaaagcaattaagtgggactggagctggatccctctgagccttacggatcagtagccagagattgttgctactaggctccgagaagagactcgaagcaggatagaccaggctacgatacaac

SEQ ID NO: 73 - DNA, 740 bp, Artificial Sequence
  Description of Artificial Sequence Synthetic oligonucleotide
  Description of Combined DNA/RNA Molecule Synthetic oligonucleotide
ggacutgcuatgcuacguguatctgaagcggagtccacacaacaagatcgggttaattgaatgctcagatggtgcgccgtctgtcctctctcttgtcctctccactatctccactacgccttaggaccagcggtgagctatgtctgaggtcacctacggacccggtttatgtgagcaaccagaattcgccttgactcgtcagcacgttattaaagctattgctatgtttgcggcggacacttatcctggtgtcgtactttcctcgtgaaatcgcgttctgcgttgtcttgggtagctatatggactagtgtatacgatcctttgaggagctagaggcggtcactgaactcctcttaatcctgacacaatatggaggtggctggctcgaatcgaccaacctgcgccgcctgagccgacctacggactactcgtgcagtgagctctttatttattagttcgcgctacctttgatacagggcaaacatactcaagctttgtattcactcgccgtcccttcaacgccgcctatgttaacgagattaatgcaaccttaggtccgcagcacacgcataagggattggtgtatgtgggcgcccaaccacaatgcgtgcgcagttagaaagcaattaagtgggactggagctggatccctctgagccttacggatcagtagccagagattgttgctactaggctccgagaagagactcgaagcaggatagaccgtagggcaatgattc

SEQ ID NO: 74 - DNA, 20 bp, Artificial Sequence
  Description of Artificial Sequence Synthetic oligonucleotide
  Description of Combined DNA/RNA Molecule Synthetic oligonucleotide
ggacttgctaugctacgtgu

SEQ ID NO: 75 - DNA, 20 bp, Artificial Sequence
  Description of Artificial Sequence Synthetic oligonucleotide
  Description of Combined DNA/RNA Molecule Synthetic oligonucleotide
gaatcatugccctacggtcu

SEQ ID NO: 76 - DNA, 20 bp, Artificial Sequence
  Description of Artificial Sequence Synthetic oligonucleotide
  Description of Combined DNA/RNA Molecule Synthetic oligonucleotide
ggacttgctaugctacgtgu

SEQ ID NO: 77 - DNA, 23 bp, Artificial Sequence
  Description of Artificial Sequence Synthetic oligonucleotide
  Description of Combined DNA/RNA Molecule Synthetic oligonucleotide
ggacttgctaugctacgtguatc

SEQ ID NO: 78 - DNA, 23 bp, Artificial Sequence
  Description of Artificial Sequence Synthetic oligonucleotide
  Description of Combined DNA/RNA Molecule Synthetic oligonucleotide
gaatcautgcccuacggtcuatc

SEQ ID NO: 79 - DNA, 12 bp, Artificial Sequence
  Description of Artificial Sequence Synthetic oligonucleotide
ttatctgaagcg

SEQ ID NO: 80 - DNA, 12 bp, Artificial Sequence
  Description of Artificial Sequence Synthetic oligonucleotide
gtatctgaagcg

SEQ ID NO: 81 - DNA, 12 bp, Artificial Sequence
  Description of Artificial Sequence Synthetic oligonucleotide
ttatcctgcttc

SEQ ID NO: 82 - DNA, 12 bp, Artificial Sequence
  Description of Artificial Sequence Synthetic oligonucleotide
ctatcctgcttc

SEQ ID NO: 83 - DNA, 20 bp, Artificial Sequence
  Description of Artificial Sequence Synthetic oligonucleotide
  Description of Combined DNA/RNA Molecule Synthetic oligonucleotide
tgttcagcgatccgtccacu

SEQ ID NO: 84 - DNA, 20 bp, Artificial Sequence
  Description of Artificial Sequence Synthetic oligonucleotide
  Description of Combined DNA/RNA Molecule Synthetic oligonucleotide
tgccacagagttccaccagu

SEQ ID NO: 85 - DNA, 20 bp, Artificial Sequence
  Description of Artificial Sequence Synthetic oligonucleotide
  Description of Combined DNA/RNA Molecule Synthetic oligonucleotide
aagccagaccgttccacaau

SEQ ID NO: 86 - DNA, 20 bp, Artificial Sequence
  Description of Artificial Sequence Synthetic oligonucleotide
  Description of Combined DNA/RNA Molecule Synthetic oligonucleotide
gccgttacagcctaatccgu

SEQ ID NO: 87 - DNA, 20 bp, Artificial Sequence
  Description of Artificial Sequence Synthetic oligonucleotide
  Description of Combined DNA/RNA Molecule Synthetic oligonucleotide
gtgccattccaactctccgu

SEQ ID NO: 88 - DNA, 20 bp, Artificial Sequence
  Description of Artificial Sequence Synthetic oligonucleotide
  Description of Combined DNA/RNA Molecule Synthetic oligonucleotide
gaagcaagcctgtctgcctu

SEQ ID NO: 89 - DNA, 20 bp, Artificial Sequence
  Description of Artificial Sequence Synthetic oligonucleotide
  Description of Combined DNA/RNA Molecule Synthetic oligonucleotide
gctctgtggacgaaggatgu

SEQ ID NO: 90 - DNA, 20 bp, Artificial Sequence
  Description of Artificial Sequence Synthetic oligonucleotide
  Description of Combined DNA/RNA Molecule Synthetic oligonucleotide
tgcaagcgtgtatgatggcu

SEQ ID NO: 91 - DNA, 22 bp, Artificial Sequence
  Description of Artificial Sequence Synthetic oligonucleotide
  Description of Combined DNA/RNA Molecule Synthetic oligonucleotide
aagctcagccuccagttgctcu

SEQ ID NO: 92 - DNA, 22 bp, Artificial Sequence
  Description of Artificial Sequence Synthetic oligonucleotide
  Description of Combined DNA/RNA Molecule Synthetic oligonucleotide
acactcagcgutccgttaccgu

SEQ ID NO: 93 - DNA, 22 bp, Artificial Sequence
  Description of Artificial Sequence Synthetic oligonucleotide
  Description of Combined DNA/RNA Molecule Synthetic oligonucleotide
ccgcacacctuagttcgacgau

SEQ ID NO: 94 - DNA, 22 bp, Artificial Sequence
  Description of Artificial Sequence Synthetic oligonucleotide
  Description of Combined DNA/RNA Molecule Synthetic oligonucleotide
cgatgccaagutcaccaccgtu

SEQ ID NO: 95 - DNA, 22 bp, Artificial Sequence
  Description of Artificial Sequence Synthetic oligonucleotide
  Description of Combined DNA/RNA Molecule Synthetic oligonucleotide
ccagccagtcuccgtcagtcau

SEQ ID NO: 96 - DNA, 22 bp, Artificial Sequence
  Description of Artificial Sequence Synthetic oligonucleotide
  Description of Combined DNA/RNA Molecule Synthetic oligonucleotide
gccgcgttatucatacagccgu

SEQ ID NO: 97 - DNA, 22 bp, Artificial Sequence
  Description of Artificial Sequence Synthetic oligonucleotide
  Description of Combined DNA/RNA Molecule Synthetic oligonucleotide
accagttccgutccacctgagu

SEQ ID NO: 98 - DNA, 22 bp, Artificial Sequence
  Description of Artificial Sequence Synthetic oligonucleotide
  Description of Combined DNA/RNA Molecule Synthetic oligonucleotide
ccttcatcgtugccaccgagau

SEQ ID NO: 99 - DNA, 24 bp, Artificial Sequence
  Description of Artificial Sequence Synthetic oligonucleotide
  Description of Combined DNA/RNA Molecule Synthetic oligonucleotide
gccacatcuctcaatcugccgtgu

SEQ ID NO: 100 - DNA, 24 bp, Artificial Sequence
  Description of Artificial Sequence Synthetic oligonucleotide
  Description of Combined DNA/RNA Molecule Synthetic oligonucleotide
ggtcaatcugccgcaauccagtcu

SEQ ID NO: 101 - DNA, 24 bp, Artificial Sequence
  Description of Artificial Sequence Synthetic oligonucleotide
  Description of Combined DNA/RNA Molecule Synthetic oligonucleotide
ccaccagtugtcagagutgtgccu

SEQ ID NO: 102 - DNA, 24 bp, Artificial Sequence
  Description of Artificial Sequence Synthetic oligonucleotide
  Description of Combined DNA/RNA Molecule Synthetic oligonucleotide
gtgaacgaugccagagutgccagu

SEQ ID NO: 103 - DNA, 24 bp, Artificial Sequence
  Description of Artificial Sequence Synthetic oligonucleotide
  Description of Combined DNA/RNA Molecule Synthetic oligonucleotide
cgccagccucaagtgtugtcatgu

SEQ ID NO: 104 - DNA, 24 bp, Artificial Sequence
  Description of Artificial Sequence Synthetic oligonucleotide
  Description of Combined DNA/RNA Molecule Synthetic oligonucleotide
cgctcaagucgcatccutcgtcau

SEQ ID NO: 105 - DNA, 24 bp, Artificial Sequence
  Description of Artificial Sequence Synthetic oligonucleotide
  Description of Combined DNA/RNA Molecule Synthetic oligonucleotide
cataagcaucgccgcaucgtaccu

SEQ ID NO: 106 - DNA, 24 bp, Artificial Sequence
  Description of Artificial Sequence Synthetic oligonucleotide
  Description of Combined DNA/RNA Molecule Synthetic oligonucleotide
gccgtctgugccgagauaaccagu

SEQ ID NO: 107 - DNA, 24 bp, Artificial Sequence
  Description of Artificial Sequence Synthetic oligonucleotide
  Description of Combined DNA/RNA Molecule Synthetic oligonucleotide
gccgaaccuccgtgaauctgaccu

SEQ ID NO: 108 - DNA, 24 bp, Artificial Sequence
  Description of Artificial Sequence Synthetic oligonucleotide
  Description of Combined DNA/RNA Molecule Synthetic oligonucleotide
gacgctaauctccgccugtgaccu

SEQ ID NO: 109 - DNA, 24 bp, Artificial Sequence
  Description of Artificial Sequence Synthetic oligonucleotide
  Description of Combined DNA/RNA Molecule Synthetic oligonucleotide
gtcaatcgucagtccaucacgccu

SEQ ID NO: 110 - DNA, 24 bp, Artificial Sequence
  Description of Artificial Sequence Synthetic oligonucleotide
  Description of Combined DNA/RNA Molecule Synthetic oligonucleotide
ccagtcagugccgtcaucagcctu

SEQ ID NO: 111 - DNA, 24 bp, Artificial Sequence
  Description of Artificial Sequence Synthetic oligonucleotide
  Description of Combined DNA/RNA Molecule Synthetic oligonucleotide
atgagcaguctaacctugccgccu

SEQ ID NO: 112 - DNA, 24 bp, Artificial Sequence
  Description of Artificial Sequence Synthetic oligonucleotide
  Description of Combined DNA/RNA Molecule Synthetic oligonucleotide
tcagcacaucatcaccugcgttgu

SEQ ID NO: 113 - DNA, 24 bp, Artificial Sequence
  Description of Artificial Sequence Synthetic oligonucleotide
  Description of Combined DNA/RNA Molecule Synthetic oligonucleotide
accgtaaguccgtctgucaccgau

SEQ ID NO: 114 - DNA, 24 bp, Artificial Sequence
  Description of Artificial Sequence Synthetic oligonucleotide
  Description of Combined DNA/RNA Molecule Synthetic oligonucleotide
gctggcgtugagtccautaaccgu

SEQ ID NO: 115 - DNA, 24 bp, Artificial Sequence
  Description of Artificial Sequence Synthetic oligonucleotide
  Description of Combined DNA/RNA Molecule Synthetic oligonucleotide
cgccgauagtgtugccgauccgau

SEQ ID NO: 116 - DNA, 24 bp, Artificial Sequence
  Description of Artificial Sequence Synthetic oligonucleotide
  Description of Combined DNA/RNA Molecule Synthetic oligonucleotide
tcgaagugccatuccgccugaccu

SEQ ID NO: 117 - DNA, 24 bp, Artificial Sequence
  Description of Artificial Sequence Synthetic oligonucleotide
  Description of Combined DNA/RNA Molecule Synthetic oligonucleotide
gcagcgucaccaucttgcucaccu

SEQ ID NO: 118 - DNA, 24 bp, Artificial Sequence
  Description of Artificial Sequence Synthetic oligonucleotide
  Description of Combined DNA/RNA Molecule Synthetic oligonucleotide
gcagcguccacauccaacuccgtu

SEQ ID NO: 119 - DNA, 24 bp, Artificial Sequence
  Description of Artificial Sequence Synthetic oligonucleotide
  Description of Combined DNA/RNA Molecule Synthetic oligonucleotide
cgagccugccatucacctugccau

SEQ ID NO: 120 - DNA, 24 bp, Artificial Sequence
  Description of Artificial Sequence Synthetic oligonucleotide
  Description of Combined DNA/RNA Molecule Synthetic oligonucleotide
aagtgauccgtgucaccgutgcgu

SEQ ID NO: 121 - DNA, 24 bp, Artificial Sequence
  Description of Artificial Sequence Synthetic oligonucleotide
  Description of Combined DNA/RNA Molecule Synthetic oligonucleotide
catccgugaccgugccgautgagu

SEQ ID NO: 122 - DNA, 24 bp, Artificial Sequence
  Description of Artificial Sequence Synthetic oligonucleotide
  Description of Combined DNA/RNA Molecule Synthetic oligonucleotide
acaaccutgccgugccagugagtu

SEQ ID NO: 123 - DNA, 24 bp, Artificial Sequence
  Description of Artificial Sequence Synthetic oligonucleotide
  Description of Combined DNA/RNA Molecule Synthetic oligonucleotide
gcacgcucgtccucacagucactu

SEQ ID NO: 124 - DNA, 24 bp, Artificial Sequence
  Description of Artificial Sequence Synthetic oligonucleotide
  Description of Combined DNA/RNA Molecule Synthetic oligonucleotide
cgcaggucaccautctcaucgccu

SEQ ID NO: 125 - DNA, 24 bp, Artificial Sequence
  Description of Artificial Sequence Synthetic oligonucleotide
  Description of Combined DNA/RNA Molecule Synthetic oligonucleotide
cgccaguccgctuccatcugacau

SEQ ID NO: 126 - DNA, 24 bp, Artificial Sequence
  Description of Artificial Sequence Synthetic oligonucleotide
  Description of Combined DNA/RNA Molecule Synthetic oligonucleotide
gctcgcuccgccutgaaguaaccu

SEQ ID NO: 127 - DNA, 24 bp, Artificial Sequence
  Description of Artificial Sequence Synthetic oligonucleotide
  Description of Combined DNA/RNA Molecule Synthetic oligonucleotide
agccgautccgtuccaccutgcau

SEQ ID NO: 128 - DNA, 24 bp, Artificial Sequence
  Description of Artificial Sequence Synthetic oligonucleotide
  Description of Combined DNA/RNA Molecule Synthetic oligonucleotide
gtccacugagccuccaccuagccu

SEQ ID NO: 129 - DNA, 24 bp, Artificial Sequence
  Description of Artificial Sequence Synthetic oligonucleotide
  Description of Combined DNA/RNA Molecule Synthetic oligonucleotide
tgccaaucatgcuccaccutgcgu

SEQ ID NO: 130 - DNA, 24 bp, Artificial Sequence
  Description of Artificial Sequence Synthetic oligonucleotide
  Description of Combined DNA/RNA Molecule Synthetic oligonucleotide
gcagccuacaccutgcctuaccgu

SEQ ID NO: 131 - DNA, 23 bp, Artificial Sequence
  Description of Artificial Sequence Synthetic oligonucleotide
cgccacggttccttatcgcagtt

SEQ ID NO: 132 - DNA, 23 bp, Artificial Sequence
  Description of Artificial Sequence Synthetic oligonucleotide
acccaccctcagcagttcacgat

SEQ ID NO: 133 - DNA, 23 bp, Artificial Sequence
  Description of Artificial Sequence Synthetic oligonucleotide
gctctgactgcgccatagactgt

SEQ ID NO: 134 - DNA, 23 bp, Artificial Sequence
  Description of Artificial Sequence Synthetic oligonucleotide
tcaccgcctctcctttgaccagt

SEQ ID NO: 135 - DNA, 23 bp, Artificial Sequence
  Description of Artificial Sequence Synthetic oligonucleotide
tcacccagtcagccgtttacagt

SEQ ID NO: 136 - DNA, 23 bp, Artificial Sequence
  Description of Artificial Sequence Synthetic oligonucleotide
cgttcgccttgcctgtagacact

SEQ ID NO: 137 - DNA, 23 bp, Artificial Sequence
  Description of Artificial Sequence Synthetic oligonucleotide
agctgcggtctgatgtcactgat

SEQ ID NO: 138 - DNA, 23 bp, Artificial Sequence
  Description of Artificial Sequence Synthetic oligonucleotide
cccccatatcgccgttcagctat

SEQ ID NO: 139 - DNA, 23 bp, Artificial Sequence
  Description of Artificial Sequence Synthetic oligonucleotide
tctccccatcgccagtcagtcat

SEQ ID NO: 140 - DNA, 23 bp, Artificial Sequence
  Description of Artificial Sequence Synthetic oligonucleotide
gcccgcccttttcaatgtgtcat

SEQ ID NO: 141 - DNA, 23 bp, Artificial Sequence
  Description of Artificial Sequence Synthetic oligonucleotide
cgccgtcctaagccctgtatgat

SEQ ID NO: 142 - DNA, 23 bp, Artificial Sequence
  Description of Artificial Sequence Synthetic oligonucleotide
cgacgccgttgaccatagatgct

SEQ ID NO: 143 - DNA, 23 bp, Artificial Sequence
  Description of Artificial Sequence Synthetic oligonucleotide
ccgattcctctcccgtgcagtat

SEQ ID NO: 144 - DNA, 23 bp, Artificial Sequence
  Description of Artificial Sequence Synthetic oligonucleotide
gccgcactttgagaatgacgctt

SEQ ID NO: 145 - DNA, 23 bp, Artificial Sequence
  Description of Artificial Sequence Synthetic oligonucleotide
gcagcccctagtcagtcgctaat

SEQ ID NO: 146 - DNA, 23 bp, Artificial Sequence
  Description of Artificial Sequence Synthetic oligonucleotide
aacctgtgttccgcctgagtgat

SEQ ID NO: 147 - DNA, 23 bp, Artificial Sequence
  Description of Artificial Sequence Synthetic oligonucleotide
cccgagagtgtgtgatgccgtat

SEQ ID NO: 148 - DNA, 23 bp, Artificial Sequence
  Description of Artificial Sequence Synthetic oligonucleotide
tggacccttgccagatgagcctt

SEQ ID NO: 149 - DNA, 23 bp, Artificial Sequence
  Description of Artificial Sequence Synthetic oligonucleotide
cgcacccttccctagtaatccgt

SEQ ID NO: 150 - DNA, 23 bp, Artificial Sequence
  Description of Artificial Sequence Synthetic oligonucleotide
gcccaccctacagagttgaccct

SEQ ID NO: 151 - DNA, 23 bp, Artificial Sequence
  Description of Artificial Sequence Synthetic oligonucleotide
cccccctttaccccttgccagat

SEQ ID NO: 152 - DNA, 23 bp, Artificial Sequence
  Description of Artificial Sequence Synthetic oligonucleotide
gacccttctgtgccttgacgact

SEQ ID NO: 153 - DNA, 23 bp, Artificial Sequence
  Description of Artificial Sequence Synthetic oligonucleotide
tcgtccactgtcccctaatggct

SEQ ID NO: 154 - DNA, 23 bp, Artificial Sequence
  Description of Artificial Sequence Synthetic oligonucleotide
cccgactatcctgcgtgaaccat

SEQ ID NO: 155 - DNA, 23 bp, Artificial Sequence
  Description of Artificial Sequence Synthetic oligonucleotide
tcaacccctgtaagatggcccgt

SEQ ID NO: 156 - DNA, 23 bp, Artificial Sequence
  Description of Artificial Sequence Synthetic oligonucleotide
ccactggttgttcactgctcggt

SEQ ID NO: 157 - DNA, 23 bp, Artificial Sequence
  Description of Artificial Sequence Synthetic oligonucleotide
tgacactgtacacgattggccct

SEQ ID NO: 158 - DNA, 23 bp, Artificial Sequence
  Description of Artificial Sequence Synthetic oligonucleotide
ggttactctctcggctcgcgtat

SEQ ID NO: 159 - DNA, 23 bp, Artificial Sequence
  Description of Artificial Sequence Synthetic oligonucleotide
tgagggtttcatccgtgtcgcat

SEQ ID NO: 160 - DNA, 23 bp, Artificial Sequence
  Description of Artificial Sequence Synthetic oligonucleotide
cccctatgtcatgagtgcccgat

SEQ ID NO: 161 - DNA, 24 bp, Artificial Sequence
  Description of Artificial Sequence Synthetic oligonucleotide
tgctatgcgtgtccatgtaccgtt

SEQ ID NO: 162 - DNA, 24 bp, Artificial Sequence
  Description of Artificial Sequence Synthetic oligonucleotide
gccaatccgtgtccgtgtaacttt

SEQ ID NO: 163 - DNA, 24 bp, Artificial Sequence
  Description of Artificial Sequence Synthetic oligonucleotide
aacgcttggggtggaactcttagt

SEQ ID NO: 164 - DNA, 24 bp, Artificial Sequence
  Description of Artificial Sequence Synthetic oligonucleotide
gtcgatgcccctccagctatgaat

SEQ ID NO: 165 - DNA, 24 bp, Artificial Sequence
  Description of Artificial Sequence Synthetic oligonucleotide
ccccgtcacctttggcttatcagt

SEQ ID NO: 166 - DNA, 24 bp, Artificial Sequence
  Description of Artificial Sequence Synthetic oligonucleotide
gccccttcgactgaactttacggt

SEQ ID NO: 167 - DNA, 24 bp, Artificial Sequence
  Description of Artificial Sequence Synthetic oligonucleotide
ggccctgagtatggacctagctgt

SEQ ID NO: 168 - DNA, 24 bp, Artificial Sequence
  Description of Artificial Sequence Synthetic oligonucleotide
tgccgtccaagtctcagtctcagt

SEQ ID NO: 169 - DNA, 24 bp, Artificial Sequence
  Description of Artificial Sequence Synthetic oligonucleotide
gcaggttatcatcccagtgacgct

SEQ ID NO: 170 - DNA, 24 bp, Artificial Sequence
  Description of Artificial Sequence Synthetic oligonucleotide
cggtgtcatggtgaagatcaggct

SEQ ID NO: 171 - DNA, 24 bp, Artificial Sequence
  Description of Artificial Sequence Synthetic oligonucleotide
gccagttgccatcagaatcccagt

SEQ ID NO: 172 - DNA, 24 bp, Artificial Sequence
  Description of Artificial Sequence Synthetic oligonucleotide
acgtttcgcactcttggtagcact

SEQ ID NO: 173 - DNA, 24 bp, Artificial Sequence
  Description of Artificial Sequence Synthetic oligonucleotide
atgtgtgtaggtcaagatgcccgt

SEQ ID NO: 174 - DNA, 24 bp, Artificial Sequence
  Description of Artificial Sequence Synthetic oligonucleotide
ccccattcgtttcagcgtaccagt

SEQ ID NO: 175 - DNA, 24 bp, Artificial Sequence
  Description of Artificial Sequence Synthetic oligonucleotide
ctcccttaggtttggcatcgcagt

SEQ ID NO: 176 - DNA, 24 bp, Artificial Sequence
  Description of Artificial Sequence Synthetic oligonucleotide
cgcagtttccctttagatcgccgt

SEQ ID NO: 177 - DNA, 24 bp, Artificial Sequence
  Description of Artificial Sequence Synthetic oligonucleotide
cttgttgagcctgtgagtgaccct

SEQ ID NO: 178 - DNA, 24 bp, Artificial Sequence
  Description of Artificial Sequence Synthetic oligonucleotide
ttaccttaccctcggactcagcgt

SEQ ID NO: 179 - DNA, 23 bp, Artificial Sequence
  Description of Artificial Sequence Synthetic oligonucleotide
cgcccccgttcctaatcagttct

SEQ ID NO: 180 - DNA, 23 bp, Artificial Sequence
  Description of Artificial Sequence Synthetic oligonucleotide
gccagtcctcctcagtttaccgt

SEQ ID NO: 181 - DNA, 23 bp, Artificial Sequence
  Description of Artificial Sequence Synthetic oligonucleotide
cctgcccgtatcgcctgtaagtt

SEQ ID NO: 182 - DNA, 23 bp, Artificial Sequence
  Description of Artificial Sequence Synthetic oligonucleotide
acgccgcctgttatatgaagcct

SEQ ID NO: 183 - DNA, 23 bp, Artificial Sequence
  Description of Artificial Sequence Synthetic oligonucleotide
cgccagccttgtcagtcttgcat

SEQ ID NO: 184 - DNA, 23 bp, Artificial Sequence
  Description of Artificial Sequence Synthetic oligonucleotide
gccgaccctctgaattatgccgt

SEQ ID NO: 185 - DNA, 23 bp, Artificial Sequence
  Description of Artificial Sequence Synthetic oligonucleotide
agggggtttgtgccattgtcgat

SEQ ID NO: 186 - DNA, 23 bp, Artificial Sequence
  Description of Artificial Sequence Synthetic oligonucleotide
agcttgcctcgtcagtcagtctt

SEQ ID NO: 187 - DNA, 23 bp, Artificial Sequence
  Description of Artificial Sequence Synthetic oligonucleotide
cgcacccgtcaacaatcgcttat

SEQ ID NO: 188 - DNA, 23 bp, Artificial Sequence
  Description of Artificial Sequence Synthetic oligonucleotide
tgtgccagtcccgaatccagagt

SEQ ID NO: 189 - DNA, 23 bp, Artificial Sequence
  Description of Artificial Sequence Synthetic oligonucleotide
gaccgctctcccagattgacact

SEQ ID NO: 190 - DNA, 23 bp, Artificial Sequence
  Description of Artificial Sequence Synthetic oligonucleotide
accgaccctttccgatgttccat

SEQ ID NO: 191 - DNA, 23 bp, Artificial Sequence
  Description of Artificial Sequence Synthetic oligonucleotide
cctgatgctcccgtgtgacaact

SEQ ID NO: 192 - DNA, 23 bp, Artificial Sequence
  Description of Artificial Sequence Synthetic oligonucleotide
tgggacgctgctaggtagacact

SEQ ID NO: 193 - DNA, 23 bp, Artificial Sequence
  Description of Artificial Sequence Synthetic oligonucleotide
gcagaccgtaccttgtagaccgt

SEQ ID NO: 194 - DNA, 23 bp, Artificial Sequence
  Description of Artificial Sequence Synthetic oligonucleotide
agacaccctccgaagttgccgat

SEQ ID NO: 195 - DNA, 23 bp, Artificial Sequence
  Description of Artificial Sequence Synthetic oligonucleotide
acgtagcttcccccctgatgagt

SEQ ID NO: 196 - DNA, 23 bp, Artificial Sequence
  Description of Artificial Sequence Synthetic oligonucleotide
gaggactttgccccgtgagactt

SEQ ID NO: 197 - DNA, 23 bp, Artificial Sequence
  Description of Artificial Sequence Synthetic oligonucleotide
gccactcattgacaatacgccgt

SEQ ID NO: 198 - DNA, 23 bp, Artificial Sequence
  Description of Artificial Sequence Synthetic oligonucleotide
gttcacagtccccaatgagccct

SEQ ID NO: 199 - DNA, 23 bp, Artificial Sequence
  Description of Artificial Sequence Synthetic oligonucleotide
cccaggacttgtgaattgcccgt

SEQ ID NO: 200 - DNA, 23 bp, Artificial Sequence
  Description of Artificial Sequence Synthetic oligonucleotide
gggggcgtttactgataccgact

SEQ ID NO: 201 - DNA, 23 bp, Artificial Sequence
  Description of Artificial Sequence Synthetic oligonucleotide
actgcacatccctcgtgaacctt

SEQ ID NO: 202 - DNA, 23 bp, Artificial Sequence
  Description of Artificial Sequence Synthetic oligonucleotide
atgcctcctcctgactgaccgtt

SEQ ID NO: 203 - DNA, 23 bp, Artificial Sequence
  Description of Artificial Sequence Synthetic oligonucleotide
caacgtgctttccgatcccgtgt

SEQ ID NO: 204 - DNA, 23 bp, Artificial Sequence
  Description of Artificial Sequence Synthetic oligonucleotide
ggggtaggtcactggtcgctcat

SEQ ID NO: 205 - DNA, 23 bp, Artificial Sequence
  Description of Artificial Sequence Synthetic oligonucleotide
ctgttgtatcccacctgacggct

SEQ ID NO: 206 - DNA, 23 bp, Artificial Sequence
  Description of Artificial Sequence Synthetic oligonucleotide
cgtctacatccaaggtccgcact

SEQ ID NO: 207 - DNA, 23 bp, Artificial Sequence
  Description of Artificial Sequence Synthetic oligonucleotide
ggatcgtgtgtgtagtccagcgt

SEQ ID NO: 208 - DNA, 23 bp, Artificial Sequence
  Description of Artificial Sequence Synthetic oligonucleotide
gtgtagaatgaccagtgccccgt

SEQ ID NO: 209 - DNA, 24 bp, Artificial Sequence
  Description of Artificial Sequence Synthetic oligonucleotide
agccgtcgtcctctaagtcactgt

SEQ ID NO: 210 - DNA, 24 bp, Artificial Sequence
  Description of Artificial Sequence Synthetic oligonucleotide
agtgctgaacctccttgtgaacct

SEQ ID NO: 211 - DNA, 24 bp, Artificial Sequence
  Description of Artificial Sequence Synthetic oligonucleotide
ggactttgggctcggactcttgat

SEQ ID NO: 212 - DNA, 24 bp, Artificial Sequence
  Description of Artificial Sequence Synthetic oligonucleotide
atccctttgcctgcctatcggaat

SEQ ID NO: 213 - DNA, 24 bp, Artificial Sequence
  Description of Artificial Sequence Synthetic oligonucleotide
cggtcttagcgtccagatgtcact

SEQ ID NO: 214 - DNA, 24 bp, Artificial Sequence
  Description of Artificial Sequence Synthetic oligonucleotide
ccggctcccatttgattttgcgat

SEQ ID NO: 215 - DNA, 24 bp, Artificial Sequence
  Description of Artificial Sequence Synthetic oligonucleotide
ccccgtagtgttaccgttccgaat

SEQ ID NO: 216 - DNA, 24 bp, Artificial Sequence
  Description of Artificial Sequence Synthetic oligonucleotide
accgctctccatatcaatcgcagt

SEQ ID NO: 217 - DNA, 24 bp, Artificial Sequence
  Description of Artificial Sequence Synthetic oligonucleotide
ggcattccctctcttcgtagccat

SEQ ID NO: 218 - DNA, 24 bp, Artificial Sequence
  Description of Artificial Sequence Synthetic oligonucleotide
ccccctactcgttacactggcatt

SEQ ID NO: 219 - DNA, 24 bp, Artificial Sequence
  Description of Artificial Sequence Synthetic oligonucleotide
gcttgtccctttcccaatccgagt

SEQ ID NO: 220 - DNA, 24 bp, Artificial Sequence
  Description of Artificial Sequence Synthetic oligonucleotide
agccgttcttgttcctgtcgcaat

SEQ ID NO: 221 - DNA, 24 bp, Artificial Sequence
  Description of Artificial Sequence Synthetic oligonucleotide
caccctcttagtcccagtccaggt

SEQ ID NO: 222 - DNA, 24 bp, Artificial Sequence
  Description of Artificial Sequence Synthetic oligonucleotide
catagtcgcagttcccgtcagagt

SEQ ID NO: 223 - DNA, 24 bp, Artificial Sequence
  Description of Artificial Sequence Synthetic oligonucleotide
attggtctcgctctcagtcaggct

SEQ ID NO: 224 - DNA, 24 bp, Artificial Sequence
  Description of Artificial Sequence Synthetic oligonucleotide
atgactggctttgaccgtggacat

SEQ ID NO: 225 - DNA, 24 bp, Artificial Sequence
  Description of Artificial Sequence Synthetic oligonucleotide
aagcctgccgatatgcctgagact

SEQ ID NO: 226 - DNA, 24 bp, Artificial Sequence
  Description of Artificial Sequence Synthetic oligonucleotide
gcacatcatcgtcaggctcacagt

SEQ ID NO: 227 - DNA, 23 bp, Artificial Sequence
  Description of Artificial Sequence Synthetic oligonucleotide
  Description of Combined DNA/RNA Molecule Synthetic oligonucleotide
cgccacggutccttaucgcagtu

SEQ ID NO: 228 - DNA, 23 bp, Artificial Sequence
  Description of Artificial Sequence Synthetic oligonucleotide
  Description of Combined DNA/RNA Molecule Synthetic oligonucleotide
acccacccucagcagutcacgau

SEQ ID NO: 229 - DNA, 23 bp, Artificial Sequence
  Description of Artificial Sequence Synthetic oligonucleotide
  Description of Combined DNA/RNA Molecule Synthetic oligonucleotide
gctctgacugcgccauagactgu

SEQ ID NO: 230 - DNA, 23 bp, Artificial Sequence
  Description of Artificial Sequence Synthetic oligonucleotide
  Description of Combined DNA/RNA Molecule Synthetic oligonucleotide
tcaccgccuctccttugaccagu

SEQ ID NO: 231 - DNA, 23 bp, Artificial Sequence
  Description of Artificial Sequence Synthetic oligonucleotide
  Description of Combined DNA/RNA Molecule Synthetic oligonucleotide
tcacccagucagccguttacagu

SEQ ID NO: 232 - DNA, 23 bp, Artificial Sequence
  Description of Artificial Sequence Synthetic oligonucleotide
  Description of Combined DNA/RNA Molecule Synthetic oligonucleotide
cgttcgccutgcctguagacacu

SEQ ID NO: 233 - DNA, 23 bp, Artificial Sequence
  Description of Artificial Sequence Synthetic oligonucleotide
  Description of Combined DNA/RNA Molecule Synthetic oligonucleotide
agctgcgguctgatgucactgau

SEQ ID NO: 234 - DNA, 23 bp, Artificial Sequence
  Description of Artificial Sequence Synthetic oligonucleotide
  Description of Combined DNA/RNA Molecule Synthetic oligonucleotide
cccccataucgccgtucagctau

SEQ ID NO: 235 - DNA, 23 bp, Artificial Sequence
  Description of Artificial Sequence Synthetic oligonucleotide
  Description of Combined DNA/RNA Molecule Synthetic oligonucleotide
tctccccaucgccagucagtcau

SEQ ID NO: 236 - DNA, 23 bp, Artificial Sequence
  Description of Artificial Sequence Synthetic oligonucleotide
  Description of Combined DNA/RNA Molecule Synthetic oligonucleotide
gcccgcccutttcaaugtgtcau

SEQ ID NO: 237 - DNA, 23 bp, Artificial Sequence
  Description of Artificial Sequence Synthetic oligonucleotide
  Description of Combined DNA/RNA Molecule Synthetic oligonucleotide
cgccgtccuaagcccugtatgau

SEQ ID NO: 238 - DNA, 23 bp, Artificial Sequence
  Description of Artificial Sequence Synthetic oligonucleotide
  Description of Combined DNA/RNA Molecule Synthetic oligonucleotide
cgacgccgutgaccauagatgcu

SEQ ID NO: 239 - DNA, 23 bp, Artificial Sequence
  Description of Artificial Sequence Synthetic oligonucleotide
  Description of Combined DNA/RNA Molecule Synthetic oligonucleotide
ccgattccuctcccgugcagtau

SEQ ID NO: 240 - DNA, 23 bp, Artificial Sequence
  Description of Artificial Sequence Synthetic oligonucleotide
  Description of Combined DNA/RNA Molecule Synthetic oligonucleotide
gccgcactutgagaaugacgctu

SEQ ID NO: 241 - DNA, 23 bp, Artificial Sequence
  Description of Artificial Sequence Synthetic oligonucleotide
  Description of Combined DNA/RNA Molecule Synthetic oligonucleotide
gcagccccuagtcagucgctaau

SEQ ID NO: 242 - DNA, 23 bp, Artificial Sequence
  Description of Artificial Sequence Synthetic oligonucleotide
  Description of Combined DNA/RNA Molecule Synthetic oligonucleotide
aacctgtgutccgccugagtgau

SEQ ID NO: 243 - DNA, 23 bp, Artificial Sequence
  Description of Artificial Sequence Synthetic oligonucleotide
  Description of Combined DNA/RNA Molecule Synthetic oligonucleotide
cccgagagugtgtgaugccgtau

SEQ ID NO: 244 - DNA, 23 bp, Artificial Sequence
  Description of Artificial Sequence Synthetic oligonucleotide
  Description of Combined DNA/RNA Molecule Synthetic oligonucleotide
tggaccctugccagaugagcctu

SEQ ID NO: 245 - DNA, 23 bp, Artificial Sequence
  Description of Artificial Sequence Synthetic oligonucleotide
  Description of Combined DNA/RNA Molecule Synthetic oligonucleotide
cgcaccctuccctaguaatccgu

SEQ ID NO: 246 - DNA, 23 bp, Artificial Sequence
  Description of Artificial Sequence Synthetic oligonucleotide
  Description of Combined DNA/RNA Molecule Synthetic oligonucleotide
gcccacccuacagagutgacccu

SEQ ID NO: 247 - DNA, 23 bp, Artificial Sequence
  Description of Artificial Sequence Synthetic oligonucleotide
  Description of Combined DNA/RNA Molecule Synthetic oligonucleotide
ccccccttuacccctugccagau

SEQ ID NO: 248 - DNA, 23 bp, Artificial Sequence
  Description of Artificial Sequence Synthetic oligonucleotide
  Description of Combined DNA/RNA Molecule Synthetic oligonucleotide
gacccttcugtgcctugacgacu

SEQ ID NO: 249 - DNA, 23 bp, Artificial Sequence
  Description of Artificial Sequence Synthetic oligonucleotide
  Description of Combined DNA/RNA Molecule Synthetic oligonucleotide
tcgtccacugtccccuaatggcu

SEQ ID NO: 250 - DNA, 23 bp, Artificial Sequence
  Description of Artificial Sequence Synthetic oligonucleotide
  Description of Combined DNA/RNA Molecule Synthetic oligonucleotide
cccgactaucctgcgugaaccau

SEQ ID NO: 251 - DNA, 23 bp, Artificial Sequence
  Description of Artificial Sequence Synthetic oligonucleotide
  Description of Combined DNA/RNA Molecule Synthetic oligonucleotide
tcaaccccugtaagauggcccgu

SEQ ID NO: 252 - DNA, 23 bp, Artificial Sequence
  Description of Artificial Sequence Synthetic oligonucleotide
  Description of Combined DNA/RNA Molecule Synthetic oligonucleotide
ccactggtugttcacugctcggu

SEQ ID NO: 253 - DNA, 23 bp, Artificial Sequence
  Description of Artificial Sequence Synthetic oligonucleotide
  Description of Combined DNA/RNA Molecule Synthetic oligonucleotide
tgacactguacacgautggcccu

SEQ ID NO: 254 - DNA, 23 bp, Artificial Sequence
  Description of Artificial Sequence Synthetic oligonucleotide
  Description of Combined DNA/RNA Molecule Synthetic oligonucleotide
ggttactcuctcggcucgcgtau

SEQ ID NO: 255 - DNA, 23 bp, Artificial Sequence
  Description of Artificial Sequence Synthetic oligonucleotide
  Description of Combined DNA/RNA Molecule Synthetic oligonucleotide
tgagggttucatccgugtcgcau

SEQ ID NO: 256 - DNA, 23 bp, Artificial Sequence
  Description of Artificial Sequence Synthetic oligonucleotide
  Description of Combined DNA/RNA Molecule Synthetic oligonucleotide
cccctatgucatgagugcccgau

SEQ ID NO: 257 - DNA, 24 bp, Artificial Sequence
  Description of Artificial Sequence Synthetic oligonucleotide
  Description of Combined DNA/RNA Molecule Synthetic oligonucleotide
tgctaugcgtguccatguaccgtu

SEQ ID NO: 258 - DNA, 24 bp, Artificial Sequence
  Description of Artificial Sequence Synthetic oligonucleotide
  Description of Combined DNA/RNA Molecule Synthetic oligonucleotide
gccaauccgtguccgtguaacttu

SEQ ID NO: 259 - DNA, 24 bp, Artificial Sequence
  Description of Artificial Sequence Synthetic oligonucleotide
  Description of Combined DNA/RNA Molecule Synthetic oligonucleotide
aacgcutgggguggaacucttagu

SEQ ID NO: 260 - DNA, 24 bp, Artificial Sequence
  Description of Artificial Sequence Synthetic oligonucleotide
  Description of Combined DNA/RNA Molecule Synthetic oligonucleotide
gtcgaugccccuccagcuatgaau

SEQ ID NO: 261 - DNA, 24 bp, Artificial Sequence
  Description of Artificial Sequence Synthetic oligonucleotide
  Description of Combined DNA/RNA Molecule Synthetic oligonucleotide
ccccgucacctutggctuatcagu

SEQ ID NO: 262 - DNA, 24 bp, Artificial Sequence
  Description of Artificial Sequence Synthetic oligonucleotide
  Description of Combined DNA/RNA Molecule Synthetic oligonucleotide
gccccutcgacugaactutacggu

SEQ ID NO: 263 - DNA, 24 bp, Artificial Sequence
  Description of Artificial Sequence Synthetic oligonucleotide
  Description of Combined DNA/RNA Molecule Synthetic oligonucleotide
ggcccugagtauggaccuagctgu

SEQ ID NO: 264 - DNA, 24 bp, Artificial Sequence
  Description of Artificial Sequence Synthetic oligonucleotide
  Description of Combined DNA/RNA Molecule Synthetic oligonucleotide
tgccguccaaguctcaguctcagu

SEQ ID NO: 265 - DNA, 24 bp, Artificial Sequence
  Description of Artificial Sequence Synthetic oligonucleotide
  Description of Combined DNA/RNA Molecule Synthetic oligonucleotide
gcaggutatcaucccagugacgcu

SEQ ID NO: 266 - DNA, 24 bp, Artificial Sequence
  Description of Artificial Sequence Synthetic oligonucleotide
  Description of Combined DNA/RNA Molecule Synthetic oligonucleotide
cggtgucatggugaagaucaggcu

SEQ ID NO: 267 - DNA, 24 bp, Artificial Sequence
  Description of Artificial Sequence Synthetic oligonucleotide
  Description of Combined DNA/RNA Molecule Synthetic oligonucleotide
gccagutgccaucagaaucccagu

SEQ ID NO: 268 - DNA, 24 bp, Artificial Sequence
  Description of Artificial Sequence Synthetic oligonucleotide
  Description of Combined DNA/RNA Molecule Synthetic oligonucleotide
acgttucgcacucttgguagcacu

SEQ ID NO: 269 - DNA, 24 bp, Artificial Sequence
  Description of Artificial Sequence Synthetic oligonucleotide
  Description of Combined DNA/RNA Molecule Synthetic oligonucleotide
atgtgugtaggucaagaugcccgu

SEQ ID NO: 270 - DNA, 24 bp, Artificial Sequence
  Description of Artificial Sequence Synthetic oligonucleotide
  Description of Combined DNA/RNA Molecule Synthetic oligonucleotide
ccccautcgttucagcguaccagu

SEQ ID NO: 271 - DNA, 24 bp, Artificial Sequence
  Description of Artificial Sequence Synthetic oligonucleotide
  Description of Combined DNA/RNA Molecule Synthetic oligonucleotide
ctcccutaggtutggcaucgcagu

SEQ ID NO: 272 - DNA, 24 bp, Artificial Sequence
  Description of Artificial Sequence Synthetic oligonucleotide
  Description of Combined DNA/RNA Molecule Synthetic oligonucleotide
cgcaguttcccuttagaucgccgu

SEQ ID NO: 273 - DNA, 24 bp, Artificial Sequence
  Description of Artificial Sequence Synthetic oligonucleotide
  Description of Combined DNA/RNA Molecule Synthetic oligonucleotide
cttgtugagccugtgagugacccu

SEQ ID NO: 274 - DNA, 24 bp, Artificial Sequence
  Description of Artificial Sequence Synthetic oligonucleotide
  Description of Combined DNA/RNA Molecule Synthetic oligonucleotide
ttaccutacccucggacucagcgu

SEQ ID NO: 275 - DNA, 23 bp, Artificial Sequence
  Description of Artificial Sequence Synthetic oligonucleotide
  Description of Combined DNA/RNA Molecule Synthetic oligonucleotide
cgcccccgutcctaaucagttcu

SEQ ID NO: 276 - DNA, 23 bp, Artificial Sequence
  Description of Artificial Sequence Synthetic oligonucleotide
  Description of Combined DNA/RNA Molecule Synthetic oligonucleotide
gccagtccucctcaguttaccgu

SEQ ID NO: 277 - DNA, 23 bp, Artificial Sequence
  Description of Artificial Sequence Synthetic oligonucleotide
  Description of Combined DNA/RNA Molecule Synthetic oligonucleotide
cctgcccguatcgccugtaagtu

SEQ ID NO: 278 - DNA, 23 bp, Artificial Sequence
  Description of Artificial Sequence Synthetic oligonucleotide
  Description of Combined DNA/RNA Molecule Synthetic oligonucleotide
acgccgccugttataugaagccu

SEQ ID NO: 279 - DNA, 23 bp, Artificial Sequence
  Description of Artificial Sequence Synthetic oligonucleotide
  Description of Combined DNA/RNA Molecule Synthetic oligonucleotide
cgccagccutgtcagucttgcau

SEQ ID NO: 280 - DNA, 23 bp, Artificial Sequence
  Description of Artificial Sequence Synthetic oligonucleotide
  Description of Combined DNA/RNA Molecule Synthetic oligonucleotide
gccgacccuctgaatuatgccgu

SEQ ID NO: 281 - DNA, 23 bp, Artificial Sequence
  Description of Artificial Sequence Synthetic oligonucleotide
  Description of Combined DNA/RNA Molecule Synthetic oligonucleotide
agggggttugtgccautgtcgau

SEQ ID NO: 282 - DNA, 23 bp, Artificial Sequence
  Description of Artificial Sequence Synthetic oligonucleotide
  Description of Combined DNA/RNA Molecule Synthetic oligonucleotide
agcttgccucgtcagucagtctu

SEQ ID NO: 283 - DNA, 23 bp, Artificial Sequence
  Description of Artificial Sequence Synthetic oligonucleotide
  Description of Combined DNA/RNA Molecule Synthetic oligonucleotide
cgcacccgucaacaaucgcttau

SEQ ID NO: 284 - DNA, 23 bp, Artificial Sequence
  Description of Artificial Sequence Synthetic oligonucleotide
  Description of Combined DNA/RNA Molecule Synthetic oligonucleotide
tgtgccagucccgaauccagagu

SEQ ID NO: 285 - DNA, 23 bp, Artificial Sequence
  Description of Artificial Sequence Synthetic oligonucleotide
  Description of Combined DNA/RNA Molecule Synthetic oligonucleotide
gaccgctcucccagautgacacu

SEQ ID NO: 286 - DNA, 23 bp, Artificial Sequence
  Description of Artificial Sequence Synthetic oligonucleotide
  Description of Combined DNA/RNA Molecule Synthetic oligonucleotide
accgacccuttccgaugttccau

SEQ ID NO: 287 - DNA, 23 bp, Artificial Sequence
  Description of Artificial Sequence Synthetic oligonucleotide
  Description of Combined DNA/RNA Molecule Synthetic oligonucleotide
cctgatgcucccgtgugacaacu

SEQ ID NO: 288 - DNA, 23 bp, Artificial Sequence
  Description of Artificial Sequence Synthetic oligonucleotide
  Description of Combined DNA/RNA Molecule Synthetic oligonucleotide
tgggacgcugctagguagacacu

SEQ ID NO: 289 - DNA, 23 bp, Artificial Sequence
  Description of Artificial Sequence Synthetic oligonucleotide
  Description of Combined DNA/RNA Molecule Synthetic oligonucleotide
gcagaccguaccttguagaccgu

SEQ ID NO: 290 - DNA, 23 bp, Artificial Sequence
  Description of Artificial Sequence Synthetic oligonucleotide
  Description of Combined DNA/RNA Molecule Synthetic oligonucleotide
agacacccuccgaagutgccgau

SEQ ID NO: 291 - DNA, 23 bp, Artificial Sequence
  Description of Artificial Sequence Synthetic oligonucleotide
  Description of Combined DNA/RNA Molecule Synthetic oligonucleotide
acgtagctuccccccugatgagu

SEQ ID NO: 292 - DNA, 23 bp, Artificial Sequence
  Description of Artificial Sequence Synthetic oligonucleotide
  Description of Combined DNA/RNA Molecule Synthetic oligonucleotide
gaggacttugccccgugagactu

SEQ ID NO: 293 - DNA, 23 bp, Artificial Sequence
  Description of Artificial Sequence Synthetic oligonucleotide
  Description of Combined DNA/RNA Molecule Synthetic oligonucleotide
gccactcautgacaauacgccgu

SEQ ID NO: 294 - DNA, 23 bp, Artificial Sequence
  Description of Artificial Sequence Synthetic oligonucleotide
  Description of Combined DNA/RNA Molecule Synthetic oligonucleotide
gttcacaguccccaaugagcccu

SEQ ID NO: 295 - DNA, 23 bp, Artificial Sequence
  Description of Artificial Sequence Synthetic oligonucleotide
  Description of Combined DNA/RNA Molecule Synthetic oligonucleotide
cccaggacutgtgaautgcccgu

SEQ ID NO: 296 - DNA, 23 bp, Artificial Sequence
  Description of Artificial Sequence Synthetic oligonucleotide
  Description of Combined DNA/RNA Molecule Synthetic oligonucleotide
gggggcgtutactgauaccgacu

SEQ ID NO: 297 - DNA, 23 bp, Artificial Sequence
  Description of Artificial Sequence Synthetic oligonucleotide
  Description of Combined DNA/RNA Molecule Synthetic oligonucleotide
actgcacauccctcgugaacctu

SEQ ID NO: 298 - DNA, 23 bp, Artificial Sequence
  Description of Artificial Sequence Synthetic oligonucleotide
  Description of Combined DNA/RNA Molecule Synthetic oligonucleotide
atgcctccucctgacugaccgtu

SEQ ID NO: 299 - DNA, 23 bp, Artificial Sequence
  Description of Artificial Sequence Synthetic oligonucleotide
  Description of Combined DNA/RNA Molecule Synthetic oligonucleotide
caacgtgcuttccgaucccgtgu

SEQ ID NO: 300 - DNA, 23 bp, Artificial Sequence
  Description of Artificial Sequence Synthetic oligonucleotide
  Description of Combined DNA/RNA Molecule Synthetic oligonucleotide
ggggtaggucactggucgctcau

SEQ ID NO: 301 - DNA, 23 bp, Artificial Sequence
  Description of Artificial Sequence Synthetic oligonucleotide
  Description of Combined DNA/RNA Molecule Synthetic oligonucleotide
ctgttgtaucccaccugacggcu

SEQ ID NO: 302 - DNA, 23 bp, Artificial Sequence
  Description of Artificial Sequence Synthetic oligonucleotide
  Description of Combined DNA/RNA Molecule Synthetic oligonucleotide
cgtctacauccaagguccgcacu

SEQ ID NO: 303 - DNA, 23 bp, Artificial Sequence
  Description of Artificial Sequence Synthetic oligonucleotide
  Description of Combined DNA/RNA Molecule Synthetic oligonucleotide
ggatcgtgugtgtaguccagcgu

SEQ ID NO: 304 - DNA, 23 bp, Artificial Sequence
  Description of Artificial Sequence Synthetic oligonucleotide
  Description of Combined DNA/RNA Molecule Synthetic oligonucleotide
gtgtagaaugaccagugccccgu

SEQ ID NO: 305 - DNA, 24 bp, Artificial Sequence
  Description of Artificial Sequence Synthetic oligonucleotide
  Description of Combined DNA/RNA Molecule Synthetic oligonucleotide
agccgucgtccuctaagucactgu

SEQ ID NO: 306 - DNA, 24 bp, Artificial Sequence
  Description of Artificial Sequence Synthetic oligonucleotide
  Description of Combined DNA/RNA Molecule Synthetic oligonucleotide
agtgcugaaccuccttgugaaccu

SEQ ID NO: 307 - DNA, 24 bp, Artificial Sequence
  Description of Artificial Sequence Synthetic oligonucleotide
  Description of Combined DNA/RNA Molecule Synthetic oligonucleotide
ggactutgggcucggacucttgau

SEQ ID NO: 308 - DNA, 24 bp, Artificial Sequence
  Description of Artificial Sequence Synthetic oligonucleotide
  Description of Combined DNA/RNA Molecule Synthetic oligonucleotide
atcccuttgccugcctaucggaau

SEQ ID NO: 309 - DNA, 24 bp, Artificial Sequence
  Description of Artificial Sequence Synthetic oligonucleotide
  Description of Combined DNA/RNA Molecule Synthetic oligonucleotide
cggtcutagcguccagaugtcacu

SEQ ID NO: 310 - DNA, 24 bp, Artificial Sequence
  Description of Artificial Sequence Synthetic oligonucleotide
  Description of Combined DNA/RNA Molecule Synthetic oligonucleotide
ccggcucccatutgattutgcgau

SEQ ID NO: 311 - DNA, 24 bp, Artificial Sequence
  Description of Artificial Sequence Synthetic oligonucleotide
  Description of Combined DNA/RNA Molecule Synthetic oligonucleotide
ccccguagtgtuaccgtuccgaau

SEQ ID NO: 312 - DNA, 24 bp, Artificial Sequence
  Description of Artificial Sequence Synthetic oligonucleotide
  Description of Combined DNA/RNA Molecule Synthetic oligonucleotide
accgcuctccauatcaaucgcagu

SEQ ID NO: 313 - DNA, 24 bp, Artificial Sequence
  Description of Artificial Sequence Synthetic oligonucleotide
  Description of Combined DNA/RNA Molecule Synthetic oligonucleotide
ggcatuccctcucttcguagccau

SEQ ID NO: 314 - DNA, 24 bp, Artificial Sequence
  Description of Artificial Sequence Synthetic oligonucleotide
  Description of Combined DNA/RNA Molecule Synthetic oligonucleotide
cccccuactcgutacacuggcatu

SEQ ID NO: 315 - DNA, 24 bp, Artificial Sequence
  Description of Artificial Sequence Synthetic oligonucleotide
  Description of Combined DNA/RNA Molecule Synthetic oligonucleotide
gcttgucccttucccaauccgagu

SEQ ID NO: 316 - DNA, 24 bp, Artificial Sequence
  Description of Artificial Sequence Synthetic oligonucleotide
  Description of Combined DNA/RNA Molecule Synthetic oligonucleotide
agccgutcttgutcctgucgcaau

SEQ ID NO: 317 - DNA, 24 bp, Artificial Sequence
  Description of Artificial Sequence Synthetic oligonucleotide
  Description of Combined DNA/RNA Molecule Synthetic oligonucleotide
cacccucttagucccaguccaggu

SEQ ID NO: 318 - DNA, 24 bp, Artificial Sequence
  Description of Artificial Sequence Synthetic oligonucleotide
  Description of Combined DNA/RNA Molecule Synthetic oligonucleotide
catagucgcagutcccgucagagu

SEQ ID NO: 319 - DNA, 24 bp, Artificial Sequence
  Description of Artificial Sequence Synthetic oligonucleotide
  Description of Combined DNA/RNA Molecule Synthetic oligonucleotide
attgguctcgcuctcagucaggcu

SEQ ID NO: 320 - DNA, 24 bp, Artificial Sequence
  Description of Artificial Sequence Synthetic oligonucleotide
  Description of Combined DNA/RNA Molecule Synthetic oligonucleotide
atgacuggcttugaccguggacau

SEQ ID NO: 321 - DNA, 24 bp, Artificial Sequence
  Description of Artificial Sequence Synthetic oligonucleotide
  Description of Combined DNA/RNA Molecule Synthetic oligonucleotide
aagccugccgauatgccugagacu

SEQ ID NO: 322 - DNA, 24 bp, Artificial Sequence
  Description of Artificial Sequence Synthetic oligonucleotide
  Description of Combined DNA/RNA Molecule Synthetic oligonucleotide
gcacaucatcgucaggcucacagu

(19) United States
(12) Patent Application Publication
(10) Pub. No.: US 2021/0332078 A1
(43) Pub. Date: Oct. 28, 2021

(54) COMPOSITIONS AND METHODS FOR NUCLEIC ACID AMPLIFICATION

(71) Applicant: Twist Bioscience Corporation, South San Francisco, CA (US)

(72) Inventor: Cheng-Hsien WU, Burlingame, CA (US)

(21) Appl. No.: 17/370,980

(22) Filed: Jul. 8, 2021

Related U.S. Application Data
(63) Continuation of application No. 15/151,316, filed on May 10, 2016, now abandoned.
(60) Provisional application No. 62/159,893, filed on May 11, 2015.

(51) Int. Cl. C07H 21/04; C12Q 1/6853

(52) U.S. Cl. CPC C07H 21/04; C12Q 1/6853

(57) ABSTRACT

The present disclosure provides compositions, methods and systems for the efficient removal of a non-target nucleic acid sequence from a double stranded nucleic acid amplification product. A non-target nucleic acid sequence may be a primer sequence incorporated into the double stranded nucleic acid molecule during a nucleic acid synthesis or amplification reaction. The non-target nucleic acid sequence may include a nucleobases that are not canonical DNA nucleobases, such as uracil, that can be selectively removed as part of the non-target sequence removal process.

### CROSS-REFERENCE

This application is a continuation of U.S. patent application Ser. No. 15/151,316, filed May 10, 2016, which claims the benefit of U.S. Provisional Application No. 62/159,893 filed May 11, 2015, which is herein incorporated by reference in their entirety.

### SEQUENCE LISTING

The instant application contains a Sequence Listing which has been submitted electronically in ASCII format and is hereby incorporated by reference in its entirety. Said ASCII copy, created on May 6, 2016, is named 44854_711_301_SL.txt and is 105,551 bytes in size.

### BACKGROUND

Polymerase chain reaction (PCR) is a technique for amplification of deoxyribonucleic acid (DNA). Specific primers complementary to a template DNA are often used to initiate nucleotide polymerization to generate a copy of the template DNA. However, the primer sequence itself is often not wanted after amplification. A common method to remove primer sequence from an amplification product is to use restriction endonucleases to cut off the unwanted primer sequence. However, a drawback is that the endonuclease recognition sequence remains in the final product. Thus, there exists a need for an efficient and universal way to remove unwanted primer sequence from amplification products.

### SUMMARY

Provided herein are methods for method for nucleic acid amplification comprising: providing a double stranded template nucleic acid comprising a first strand and a second strand; mixing the double stranded template nucleic acid with a first primer and a second primer, wherein: the first primer comprises a first plurality of nucleobases that are not canonical DNA nucleobases, wherein each nucleobase of the first plurality of nucleobases that are not canonical DNA nucleobases is distanced from another by about 3 to about 8 nucleobases, and wherein one nucleobase of the first plurality of nucleobases that are not canonical DNA nucleobases is a 3′ terminal nucleobase bound to a preceding nucleobase in the first primer by a phosphorothioate bond, and the second primer comprises a second plurality of nucleobases that are not canonical DNA nucleobases, wherein each nucleobase of the second plurality of nucleobases that are not canonical DNA nucleobases is distanced from another by about 3 to about 8 nucleobases, and wherein one nucleobase of the second plurality of nucleobases that are not canonical DNA nucleobases is a 3′ terminal nucleobase bound to a preceding nucleobase in the second primer by a phosphorothioate bond; and amplifying the double stranded template nucleic acid by extending the first primer to form a first extension strand and extending the second primer to form a second extension strand; and removing the first primer from the first extension strand and the second primer from the second extension strand. Further provided herein are methods wherein the first plurality of nucleobases that are not canonical DNA nucleobases or the second plurality of nucleobases that are not canonical DNA nucleobases comprises 3 or 4 nucleobases. Further provided herein are methods wherein each nucleobase of the first plurality of nucleobases that are not canonical DNA nucleobases is distanced from another by about 4 to about 7 nucleobases. Further provided herein are method wherein each nucleobase of the second plurality of nucleobases that are not canonical DNA nucleobases is distanced from another by about 4 to about 7 nucleobases. Further provided herein are methods wherein the first primer and the second primer each have a total length of from about 12 to about 50 nucleobases. Further provided herein are methods wherein each nucleobase of the first plurality of nucleobases that are not canonical DNA nucleobases or each nucleobase of the second plurality of nucleobases that are not canonical DNA nucleobases is uracil, 3-meA (3-methyladenine), hypoxanthine, 8-oxoG (7,8-dihydro-8-oxoguanine), FapyG, FapyA, Tg (thymine glycol), hoU (hydroxyuracil), hmU (hydroxymethyluracil), fU (formyluracil), hoC (hydroxycytosine), fC (formylcytosine), 5-meC (5-methylcytosine), 6-meG (O6-methylguanine), 7-meG (N7-methylguanine), EC (ethenocytosine), 5-caC (5-carboxylcytosine), 2-hA, EA (ethenoadenine), 5-fU (5-fluorouracil), 3-meG (3-methylguanine), or isodialuric acid. Further provided herein are methods wherein the first plurality of nucleobases that are not canonical DNA nucleobases and the second plurality of nucleobases that are not canonical DNA nucleobases are the same type of nucleobase. Further provided herein are methods wherein each nucleobase of the first plurality of nucleobases that are not canonical DNA nucleobases and each nucleobase of the second plurality of nucleobases that are not canonical DNA nucleobases is uracil. Further provided herein are methods wherein the nucleobase preceding the 3′ terminal uracil nucleobase in the first primer is a pyrimidine. Further provided herein are methods wherein nucleobase preceding the 3′ terminal uracil nucleobase in the second primer is a pyrimidine. Further provided herein are methods wherein the pyrimidine is a cytosine. Further provided herein are methods wherein the first primer is removed from the first extension strand with an efficiency of at least about 90%, and the second primer is removed from the second extension strand with an efficiency of at least about 90%. Further provided herein are methods wherein the melting temperature of the first primer is between about 60° C. and about 66° C. Further provided herein are methods wherein the melting temperature of the second primer is between about 60° C. and about 66° C. Further provided herein are methods wherein the first primer, the second primer, or both the first primer and the second primer is between about 40% and about 60%. Further provided herein are methods wherein the first primer from the first extension strand comprises excising the first plurality of uracil nucleobases from the first primer from the first extension strand, and wherein removing the second primer from the second extension strand comprises excising the second plurality of uracil nucleobases from the second primer from the second extension strand. Further provided herein are methods wherein the first plurality of uracil nucleobases from the first primer of the first extension strand and excision of the second plurality of uracil nucleobases from the second primer of the second extension strand is achieved by treating the first extension strand and the second extension strand with a mixture of Uracil DNA glycosylase and a DNA glycosylase-lyase Endonuclease VIII. Further provided herein are methods wherein the first strand of the double stranded template nucleic acid comprises in 5′ to 3′ order: a first primer binding site complementary to the first primer, and a target nucleic acid sequence; and the second strand of the double stranded template nucleic acid comprises in 5′ to 3′ order: a second primer binding site complementary to the second primer, and a nucleic acid sequence complementary to the target nucleic acid sequence. Further provided herein are methods wherein the first strand of the double stranded template nucleic acid comprises in 5′ to 3′ order: the first primer binding site complementary to the first primer, the target nucleic acid sequence, and a nucleic acid sequence complementary to the second primer binding site; and the second strand of the double stranded template nucleic acid comprises in 5′ to 3′ order: the second primer binding site complementary to the second primer, the nucleic acid sequence complementary to the target nucleic acid sequence, and a nucleic acid sequence complementary to the first primer binding site. Further provided herein are methods wherein the first extension strand comprises in 5′ to 3′ order: the first primer, the target nucleic acid sequence, and a nucleic acid sequence complementary to the second primer; and the second extension strand comprises in 5′ to 3′ order: the second primer, the nucleic acid sequence complementary to the target nucleic acid sequence, and a nucleic acid sequence complementary to the first primer. Further provided herein are methods comprising removing from the second extension strand the nucleic acid sequence complementary to the first primer and removing from the first extension strand the nucleic acid sequence complementary to the second primer. Further provided herein are methods wherein the nucleic acid sequence complementary to the first primer and the nucleic acid sequence complementary to the second primer are removed by digestion with an enzyme comprising exonuclease activity. Further provided herein are methods wherein the enzyme comprising exonuclease activity is a DNA polymerase. Further provided herein are methods wherein each strand of the double stranded templated nucleic acid is generated by assembly of de novo synthesis. Further provided herein are methods wherein each strand of the double stranded templated nucleic acid is generated by assembly of de novo synthesis, and wherein de novo synthesis comprises synthesis of a plurality of oligonucleotides which collectively encode for a sequence of the double stranded template nucleic acid. Further provided herein are methods wherein the double stranded target nucleic acid has a length from about 160 to about 10,000 nucleobases.

Provided herein are methods for amplifying a template nucleic acid, the method comprising: providing a template nucleic acid comprising (i) a first template strand comprising in 5′ to 3′ order: a first primer binding site and a target nucleic acid, and (ii) a second template strand comprising in 5′ to 3′ order: a second primer binding site and a nucleic acid sequence complementary to the target nucleic acid; annealing a first primer to the first primer binding site, and annealing a second primer to the second primer binding site, wherein: the first primer comprises 3 or 4 uracil nucleobases, each uracil nucleobase distanced from another uracil nucleobase in the first primer by about 3 to about 8 non-uracil nucleobases, the second primer comprises 3 or 4 uracil nucleobases, each uracil nucleobase distanced from another uracil nucleobase in the second primer by about 3 to about 8 non-uracil nucleobases, one uracil nucleobase of the 3 or 4 uracil nucleobases in the first primer is a 3′ terminal uracil nucleobase bound to a preceding nucleobase in the first primer by a first nuclease resistant bond, and one uracil nucleobase of the 3 or 4 uracil nucleobases in the second primer is a 3′ terminal uracil nucleobase bound to a preceding nucleobase in the second primer by a second nuclease resistant bond; forming an amplicon nucleic acid by: extending the first primer and the second primer to generate a first extension strand comprising the first primer and the target nucleic acid, and extending the second primer to generate a second extension strand comprising the second primer and the nucleic acid sequence complementary to the target nucleic acid; and removing the first primer and the second primer from the amplicon nucleic acid. Further provided herein are methods wherein the first nuclease resistant bond or the second nuclease resistant bond is a phosphorothioate bond. Further provided herein are methods wherein the first primer or the second primer comprises 3 uracil nucleobases. Further provided herein are methods wherein the first primer or the second primer comprises 4 uracil nucleobases. Further provided herein are methods wherein the nucleobase preceding the 3′ terminal uracil nucleobase in the first primer is a pyrimidine. Further provided herein are methods wherein the nucleobase preceding the 3′ terminal uracil nucleobase in the second primer is a pyrimidine. Further provided herein are methods wherein the first primer is removed from the first extension strand with an efficiency of at least about 90%, and the second primer is removed from the second extension strand with an efficiency of at least about 90%. Further provided herein are methods wherein removing the first primer and removing the second primer comprises: excising the 3 or 4 uracil nucleobases from the first primer of the first extension strand, and excising the 3 or 4 uracil nucleobases from the second primer of the second extension strand. Further provided herein are methods wherein excision of the 3 or 4 uracil nucleobases from the first primer of the first extension strand and excision of the 3 or 4 nucleobases from the second primer of the second extension strand is achieved by treating the first extension strand and the second extension strand with a mixture of Uracil DNA glycosylase and a DNA glycosylase-lyase Endonuclease VIII. Further provided herein are methods wherein the first template strand comprises in 5′ to 3′ order: the first primer binding site, the target nucleic acid, and a nucleic acid sequence complementary to the second primer binding site; and the second template strand comprises in 5′ to 3′ order: the second primer binding site, the nucleic acid sequence complementary to the target nucleic acid, and a nucleic acid sequence complementary to the first primer binding site. Further provided herein are methods wherein the first extension strand comprises in 5′ to 3′ order: the first primer, the target nucleic acid, and a nucleic acid sequence complementary to the second primer; and the second extension strand comprises in 5′ to 3′ order: the second primer, the nucleic acid sequence complementary to the target nucleic acid, and a nucleic acid sequence complementary to the first primer. Further provided herein are methods comprising removing the nucleic acid sequence complementary to the first primer from the second extension strand, and removing the nucleic acid sequence complementary to the second primer from the first extension strand, to generate a double-stranded product comprising the target nucleic acid and the nucleic acid sequence complementary to the target nucleic acid. Further provided herein are methods wherein the nucleic acid sequence complementary to the first primer and the nucleic acid sequence complementary to the second primer are removed by digesting the first product strand and the second product strand with an enzyme comprising exonuclease activity. Further provided herein are methods wherein the enzyme comprising exonuclease activity is a DNA polymerase. Further provided herein are methods wherein the target nucleic acid has a length from about 160 to about 10,000 nucleobases.

Provided herein are nucleic acid libraries, wherein the nucleic acid libraries comprises a plurality of double-stranded nucleic acids, each of the plurality of the double-stranded nucleic acids comprising: a first strand comprising in 5′ to 3′ order: a first primer and a target nucleic acid, wherein the first primer comprises a first plurality of uracil nucleobases, wherein each uracil nucleobase of the first plurality of uracil nucleobases is distanced from another uracil nucleobase in the first primer by about 3 to about 8 non-uracil nucleobases, and wherein one uracil nucleobase of the first plurality of uracil nucleobases is a 3′ terminal uracil nucleobase bound to a preceding nucleobase in the first primer by a first nuclease resistant bond; and a second strand comprising in 5′ to 3′ order: a second primer and a nucleic acid sequence complementary to the target nucleic acid, wherein the second primer comprises a second plurality of uracil nucleobases, wherein each uracil nucleobase of the second plurality of uracil nucleobases is distanced from another uracil nucleobase in the second primer by about 3 to about 8 non-uracil nucleobases, and wherein one uracil nucleobase of the second plurality of uracil nucleobases is a 3′ terminal uracil nucleobase bound to a preceding nucleobase in the second primer by a second nuclease resistant bond. Further provided herein are libraries wherein each strand of the plurality of double-stranded nucleic acids has a length from about 160 to about 10,000 nucleobases. Further provided herein are libraries wherein the first plurality of uracil nucleobases or the second plurality of uracil nucleobases comprises 3 or 4 uracil nucleobases. Further provided herein are libraries wherein each uracil nucleobase of the first plurality of uracil nucleobases is distanced from another uracil nucleobase in the first primer by about 4 to about 7 non-uracil nucleobases. Further provided herein are libraries wherein each uracil nucleobase of the second plurality of uracil nucleobases is distanced from another uracil nucleobase in the second primer by about 4 to about 7 non-uracil nucleobases. Further provided herein are libraries wherein the nucleobase positioned immediately 5′ to the 3′ terminal uracil nucleobase of the first primer is a pyrimidine. Further provided herein are libraries wherein the nucleobase positioned immediately 5′ to the 3′ terminal uracil nucleobase of the second primer is a pyrimidine. Further provided herein are libraries wherein the pyrimidine is a cytosine nucleobase. Further provided herein are libraries wherein the first nuclease resistant bond or the second nuclease resistant bond is a phosphorothioate bond. Further provided herein are libraries wherein the length of the first primer or the second is from about 12 to about 50 nucleobases. Further provided herein are libraries wherein the plurality of double-stranded nucleic acids is at least about 200 double-stranded nucleic acids. Further provided herein are libraries wherein each first primer in the plurality of double-stranded nucleic acids comprises a first nucleic acid sequence that is the same. Further provided herein are libraries wherein each second primer in the plurality of double-stranded nucleic acids comprises a second nucleic acid sequence that is the same.

Provided herein are universal primers, each comprising: a non-naturally occurring sequence of nucleic acids comprising 3 or more uracil nucleobases; wherein one of the 3 or more uracil nucleobases is a 3′ terminal uracil nucleobase bound to a preceding nucleobase in the universal primer by a phosphorothioate bond; and wherein each of the 3 or more uracil nucleobases is distanced from a subsequent or a preceding uracil nucleobase in the universal primer by about 3 to about 8 non-uracil nucleobases. Further provided herein is a universal primer wherein the 3 or more uracil nucleobases 3 or 4 uracil nucleobases. Further provided herein is a universal primer wherein each of the 3 or more uracil nucleobases is distanced from a subsequent or a preceding uracil nucleobase in the universal primer by about 4 to about 7 non-uracil nucleobases. Further provided herein is a universal primer wherein the universal primer has a length from about 12 to about 50 nucleobases. Further provided herein is a universal primer wherein the nucleobase preceding the 3′ terminal uracil nucleobase is a pyrimidine. Further provided herein is a universal primer wherein the pyrimidine is a cytosine nucleobase. Further provided herein is a universal primer wherein the melting temperature of the universal primer is between about 60° C. and about 66° C. Further provided herein is a universal primer wherein the GC content is between about 40% and about 60%. Further provided herein is a nucleic acid amplification reaction mixture comprising a universal primer described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A depicts exemplary configurations of universal primers (SEQ ID NOS: 1-4, respectively, in order of appearance).

FIG. 1B depicts a process for nucleic acid amplification using a pair of universal primers, such as those exemplified in FIG. 1A.

FIG. 2 depicts a process for removing a nucleic acid sequence from an amplification product.

FIG. 3 depicts a process for removing a nucleic acid sequence from a dsDNA molecule comprising a plurality of uracil nucleobases.

FIG. 4 depicts a process for removing a nucleic acid sequence from a dsDNA molecule comprising a target nucleic acid, followed by repairing the ends of the target nucleic acid.

FIG. 5 depicts a process for removing a select nucleic acid sequence from a dsDNA molecule comprising a target nucleic acid, followed by an optional step for repairing ends of the target nucleic acid.

FIG. 6 depicts a process for removing a select nucleic acid sequence from a dsDNA molecule comprising uracil nucleobases.

FIG. 7 depicts a process for removing a select nucleic acid sequence from a dsDNA molecule comprising a target nucleic acid, followed by repairing the ends of the target nucleic acid.

FIG. 8 depicts a process for amplifying a product of a polymerase chain assembly reaction with universal primers, followed by removal of sequence encoding the same sequence as the universal primers from the amplification products.

FIG. 9 is a schematic for a gene synthesis process using universal primers.

FIG. 10 depicts a system comprising various storage and computing elements.

FIG. 11 depicts a computer system.

FIG. 12 depicts a computer architecture.

FIG. 13 is an image of a DNA agarose gel showing a decrease in crude amplicon size after primer removal.

FIG. 14 is an image of DNA agarose gel showing a decrease in purified amplicon size after primer removal.

FIG. 15 is a plot of primer removal efficiency as a function of enzymatic treatment time.

### DETAILED DESCRIPTION

In a variety of genetic and microbiology applications, it is often necessary to obtain a large quantity of a target nucleic acid. This is generally achieved by performing a nucleic acid amplification reaction, whereby the target nucleic acid is copied in an iterative process involving primer annealing and extension using the target nucleic acid as a template. These amplification products are then used for applications, for example DNA cloning, DNA sequencing, protein optimization, DNA-based phylogeny, and disease diagnosis. For cases where a plurality of different target nucleic acids is to be amplified in a single reaction, a universal primer pair that anneals to each of the plurality of target nucleic acids can be used in one batch amplification. In such cases, the universal primer pair anneals to shared universal primer binding sequences flanking each of the plurality of target nucleic acids. The amplification products from each of the plurality of target nucleic acids comprise a copy of a target nucleic acid sequence and a universal primer sequence. For many applications, this universal primer sequence must be completely removed from the amplification product, without altering the integrity of the target nucleic acid sequence. In various aspects, the present disclosure addresses this need by providing compositions, methods and systems to selectively remove sequence from a target nucleic acid which was added to the target nucleic acid for the sake of an amplification step. In the context of nucleic acid amplification reactions, this added sequence is sometimes referred to as an adapter sequence, where the adapter sequence is a primer binding site for the amplification of the target nucleic acid. In the same context, an adapter sequence further refers to a primer sequence of an amplification product.

In various applications, a target nucleic acid is a product of a plurality of assembled, de novo synthesized precursor nucleic acid fragments. To amplify the target nucleic acid, primer binding sequences are appended to both ends of the target nucleic acid. This addition of primer binding sequence optionally occurs during assembly of the precursor nucleic acid fragments. A method for precursor nucleic acid assembly includes a ligation reaction, such as a polymerase chain assembly (PCA) reaction. For PCA reactions, precursor nucleic acid fragments spanning the target nucleic acid each have an overlapping sequence with another precursor nucleic acid fragment in a sequence-dependent manner. During the ligation reaction, the precursor nucleic acid fragments anneal to complementary fragments, and any gaps in sequence are then filled in by addition of a polymerase and a pool of deoxynucleotides. The ligation reaction is repeated over a number of cycles as the fragments form longer and longer fragments until the target nucleic acid is formed. To ensure integrity of the desired target nucleic acid sequence, an error correction step is sometimes performed. The resulting target nucleic acid is then a candidate for amplification using the methods provided herein.

Provided herein are methods for the amplification of a target nucleic acid with a primer, wherein the primer is designed with one or more features that allow for enzymatic removal of the primer sequence after amplification. In many instances, the removal of the primer sequence is “scar-free,” where no primer sequence remains linked to an amplification product of the target sequence after primer removal, and the target sequence remaining after primer removal is the same as the template sequence from which it was copied. During an amplification reaction, a primer having one or more features is annealed to, and extended from, a primer binding sequence appended to an end of a target sequence. For some primers, one feature is a 3′ terminal uracil nucleobase that will boarder a copy of the target sequence in a resulting amplification product. The uracil is then selectively removed in a process that targets the uracil for excision to generate a gap between the remaining primer sequence and the target sequence in the amplification product. The remaining primer sequence is then removed, without disrupting the target sequence. When a pool of nucleic acids having different target sequences is designed to have a primer binding sequence shared by multiple template nucleic acids, a single universal primer pair can be used to amplify the pool to generate a diverse library. Such an arrangement reduces cost and affords efficiencies in the amplification process.

### Definitions

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

Throughout this disclosure, various embodiments can be presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed herein, subject to any specifically excluded limit in the stated range. Where the stated range includes one or both of the limits, ranges excluding either or both of those included limits are also included herein, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 15%, 14%, 13%, 12%, 11%, 10%, 9%, 8%, 7%, 6%, 5%, 4%, 3%, 2%, 1%, 0.5%, 0.1%, 0.05%, or 0.01% of the stated value.

As used herein, a nucleic acid molecule is a polynucleotide that comprises deoxyribonucleic acid (DNA), ribonucleic acid (RNA), or any combination thereof. A nucleic acid may be single stranded (ss) or double stranded (ds). A double stranded nucleic acid may possess a nick or a gap. A nucleic acid represents both a single and plural or clonally amplified population of nucleic acid molecules.

Primers

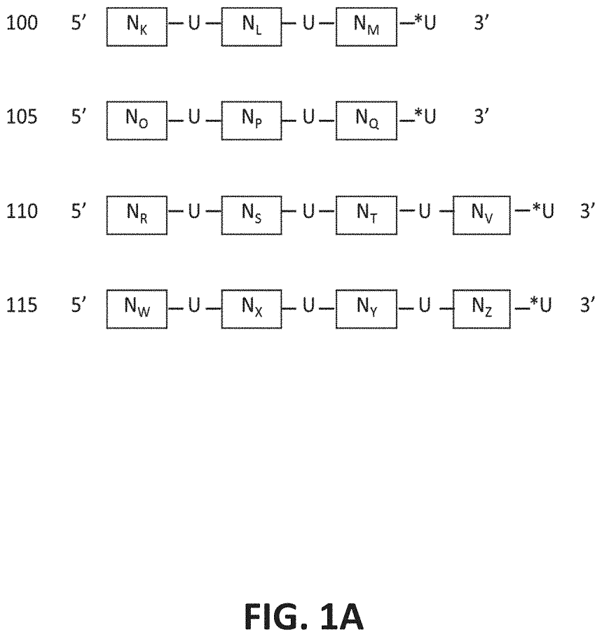

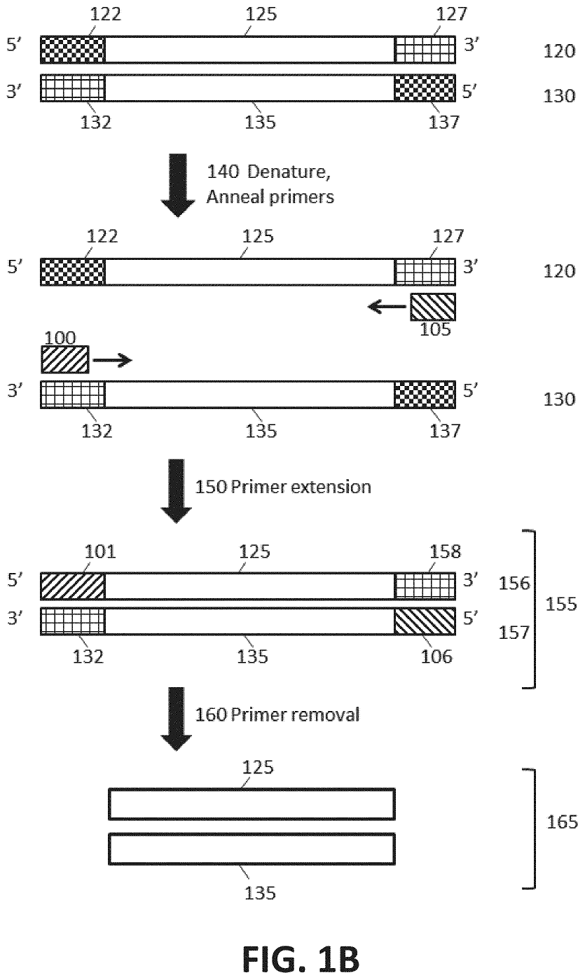

An illustration of two exemplary universal primer pairs for use in nucleic acid amplification methods is provided in FIG. 1A: forward primer 100 (SEQ ID NO. 1) and reverse primer 105 (SEQ ID NO. 2); and forward primer 110 (SEQ ID NO. 3) and reverse primer 115 (SEQ ID NO. 4). Each of these four exemplary universal primers comprise a plurality of nucleobases that are not canonical DNA nucleobases, in this case a uracil (denoted by U), which are incorporated into double-stranded amplicons during amplification of a target nucleic acid with the universal primers. Following amplification, the uracil nucleobases of the double-stranded amplicons are removable by an enzymatic process. When two uracil nucleobases from the plurality of uracil nucleobases in each universal primer are separated by one or more non-uracil nucleobases in a first strand of a double-stranded amplicon, removal of the two uracil nucleobases generates two gaps flanking the one or more non-uracil nucleobases. The one or more non-uracil nucleobases can then be removed by separating the two strands of the double-stranded amplicon. When the two strands are hybridized back together by base-pairing of target nucleic acid sequence, a second strand of the amplicon comprises an overhang complementary to the removed primer sequence. This overhang is then removable, for example, by treatment with an enzyme having exonuclease activity. A first primer pair shown in FIG. 1A comprises forward primer 100 and reverse primer 105, each primer comprising three uracil nucleobases. A second primer pair comprises forward primer 110 and reverse primer 115, each primer comprising four uracil nucleobases. The pairing of these primers is not limiting, and additional primer pairs are envisioned whereby a forward primer comprises three uracil bases and a reverse primer comprises four uracil bases, and vice versa. Further, additional primer pairs include those having any plurality of uracil nucleobases, from about two to about eight uracil nucleobases.

Universal primers used in amplification methods described herein may comprise one or more features for removal of the primers after the amplification methods are performed. For cases where a feature comprises two or more nucleobases that are targets for enzymatic removal, the two or more nucleobases are arranged within the primer sequence to facilitate efficient primer removal when the primer is incorporated into a product such as an amplicon. For the universal primers 100, 105, 110, 115, the uracil nucleobases are spaced apart by non-uracil nucleobases N. The subscripts K, L, M, O, P, Q, R, S, T, V, W, X, Y, and Z in FIG. 1A independently represent the number of non-uracil nucleobases within the primers. For any one of N_{K}, N_{L}, N_{M}, N_{O}, N_{P}, N_{Q}, N_{R}, N_{S}, N_{T}, N_{V}, N_{W}, N_{X}, N_{Y}, and N_{Z}, the number of non-uracil nucleobases is about 2-10, 3-8, or 4-7. In some cases, any one of N_{K}, N_{L}, N_{M}, N_{O}, N_{P}, N_{Q}, N_{R}, N_{S}, N_{T}, N_{V}, N_{W}, N_{X}, N_{Y}, and N_{Z}, is 2, 3, 4, 5, 6, 7, 8, 9 or 10 non-uracil nucleobases. In order to remove each nucleobase of one of the universal primers 100, 105, 110, 115 from an amplicon into which it is incorporated, one of the uracil nucleobases is a 3′ terminal uracil nucleobase. This 3′ terminal uracil nucleobase is bound to a preceding nucleobase by a nuclease resistant bond to prevent removal of the 3′ terminal uracil nucleobase during an amplification reaction with a DNA polymerase having exonuclease activity. This nuclease resistant bond is depicted with an asterisk in FIG. 1A. An example of a nuclease resistant bond is a phosphorothioate bond.

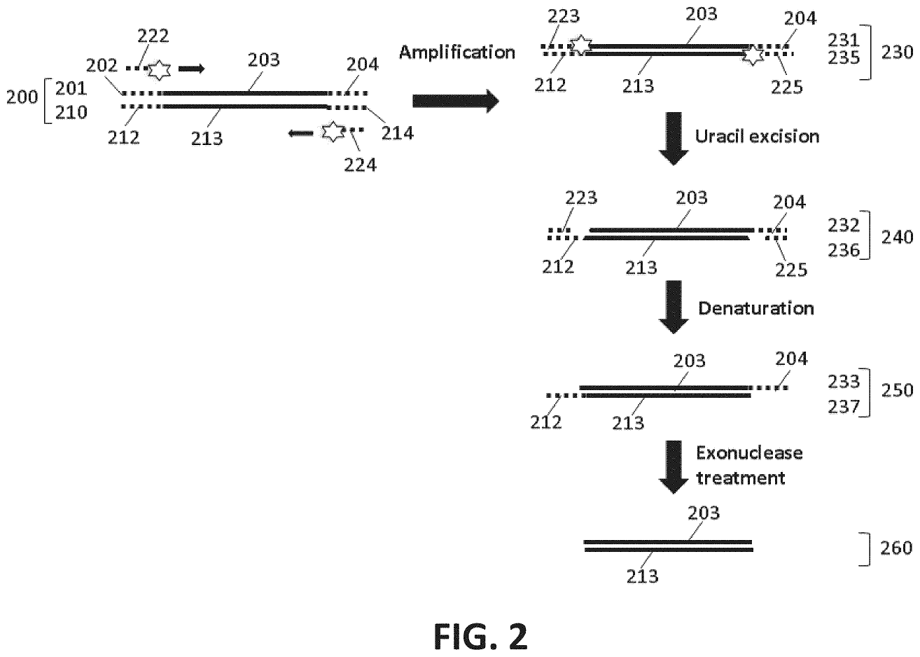

An amplification reaction incorporating universal primer pairs, e.g., those exemplified in FIG. 1A, is depicted in the process of FIG. 1B. A target nucleic acid 125 is flanked by a 5′ adapter sequence 122 and a 3′ adapter sequence 127 in a first template strand 120. Hybridized to the first template strand 120 is a second template strand 130 comprising a complementary target sequence 135 which is complementary to the target nucleic acid 125 (“target nucleic acid complement”), and flanked by a 5′ adapter sequence 137 and a 3′ adapter sequence 132. The 3′ adapter sequence 132 of the complementary target sequence 135 serves as a primer binding site for a first universal primer 100, and 3′ adapter sequence 127 of the target sequence 125 serves as a primer binding site for a second universal primer 105 (step 140). The first universal primer 100 and the second universal primer 105 anneal to the primer binding sites of the second template strand 130 and the first template strand 120, respectively, and extend in a 5′ to 3′ direction (step 150) to generate extension strands 155. A first extension strand 156 comprises: a 5′ adapter sequence encoding the same sequence as universal primer 101, a copy of the target nucleic acid 125, and a 3′ adapter sequence 158; wherein the 3′ uracil nucleobase from universal primer 100 is positioned immediately upstream and adjacent to the 5′ nucleobase of the target nucleic acid 125 copy. A second extension strand 157 comprises: a 5′ sequence encoding the same sequence as primer 106, a copy of the complementary target sequence 135, and a 3′ adapter sequence 132; wherein the 3′ uracil nucleobase from universal primer 105 is positioned immediately upstream and adjacent to the 5′ nucleobase of the complementary target sequence 135. The 5′ and 3; adapter sequences 101, 106, 132, 158 are selectively removed from the extension strands 155 in a primer removal process (step 160) to generate a product 165. Non-limiting examples of primer removal processes are described with reference to following FIGS. 2-7.

Primer Removal

An exemplary process for primer removal after target nucleic acid amplification is depicted in FIG. 2. During target nucleic acid amplification, a double-stranded DNA molecule 200 is amplified using a forward universal primer 222 and a reverse universal primer 224, each universal primer having a 3′ terminal uracil (depicted by a star in FIG. 2), to allow for removal of the universal primer sequence after amplification. Non-limiting examples of universal primer sequences applicable for use in the amplification process discussed herein are listed in Tables 2, 6, 10, 19, 22 and 27. In a first step of FIG. 2, a the forward universal primer 222 and the reverse universal primer 224 are used to amplify a double-stranded DNA molecule 200, which comprises a first nucleic acid strand 201 and a second nucleic acid strand 210. The first nucleic acid strand 201 comprises in 5′ to 3′ order: a first adapter 202, a target nucleic acid 203, and a second adapter 204. The second nucleic acid strand 210 comprises in 5′ to 3′ order: a nucleic acid sequence 214 reverse complementary to the second adapter 204 (referred to as “second adapter complement”), a nucleic acid sequence 213 reverse complementary to target nucleic acid 203 (referred to as “target nucleic acid complement”), and a nucleic acid sequence 212 reverse complementary to the first adapter 202 (referred to as “first adapter complement”).

Double-stranded DNA 200 is amplified in a reaction mixture comprising: double-stranded DNA 200, the forward universal primer 222, the reverse universal primer 224, a polymerase compatible with the uracil nucleobase of the primers, and dNTPs. To mitigate removal of the 3′ uracil nucleobases from the forward universal primer 222 and the reverse universal primer 224 during the amplification reaction by the polymerase, the 3′ terminal uracil nucleobase from each universal primer is bound to the 5′ adjacent nucleobase by a nuclease resistant bond. A product of the amplification reaction, amplicon 230, comprises a first amplicon strand 231 and a second amplicon strand 235. The first amplicon strand 231 comprises in 5′ to 3′ order: a sequence 223 encoding the same sequence as the forward universal primer 222 and comprising the 3′ uracil, a copy of target nucleic acid 203, and a copy of second adapter sequence 204. The second amplicon strand 235 comprises in 5′ to 3′ order: a sequence 225 encoding the same sequence as the reverse universal primer 224 comprising the 3′ uracil, a copy of target nucleic acid complement 213, and a copy of the first adapter sequence complement 212.

In a first step for the selective removal of sequences 223, 212, 204, 225 from the amplicon 230, the 3′ uracil nucleobase in the first amplicon strand 231 and the second amplicon strand 235 of amplicon 230 is excised using a DNA repair enzyme having both glycosylase activity specific for the uracil nucleobase and endonuclease activity. For example, amplicon 230 is treated with a UDG glycosylase and an endonuclease VIII in a cleavage reaction mixture to excise the uracil nucleobases from the first amplicon strand 231 and the second amplicon strand 235, generating a product 240. The product 240 comprises a first nicked amplicon strand 232 and a second nicked amplicon strand 236, wherein each nick is in reference to a single nucleobase gap due to excision of the 3′ uracil in the first amplicon strand 231 and the second amplicon strand 235. To remove nucleobases remaining in sequences (5′ adapter 223 of the first nicked amplicon strand 232, and 5′ adapter 225 of the second nicked amplicon strand 236), which are 5′ in their respective strands to the single nucleobase gap, product 240 is denatured and then annealed to generate product 250. The double-stranded overhang product 250 comprises a first strand 233 having a 3′ overhang with sequence 204, and a second strand 237 having a 3′ overhang with sequence 237. The 3′ overhangs of product 240, are removed using a polymerase having exonuclease activity under denaturing conditions (e.g., temperatures greater than about 60° C. or between about 60° C. and about 100° C.). The resulting product lacking adapter sequence 260 comprises a copy of the target nucleic acid 203 and a copy of the target nucleic acid complement 213. Thus, the process of FIG. 2 illustrates a method for generating an amplification product 260 of a target nucleic acid 203 using a pair of universal primers that are removable without disrupting the sequence of the target nucleic acid 203, and without the addition of extraneous sequence to the target nucleic acid 203.

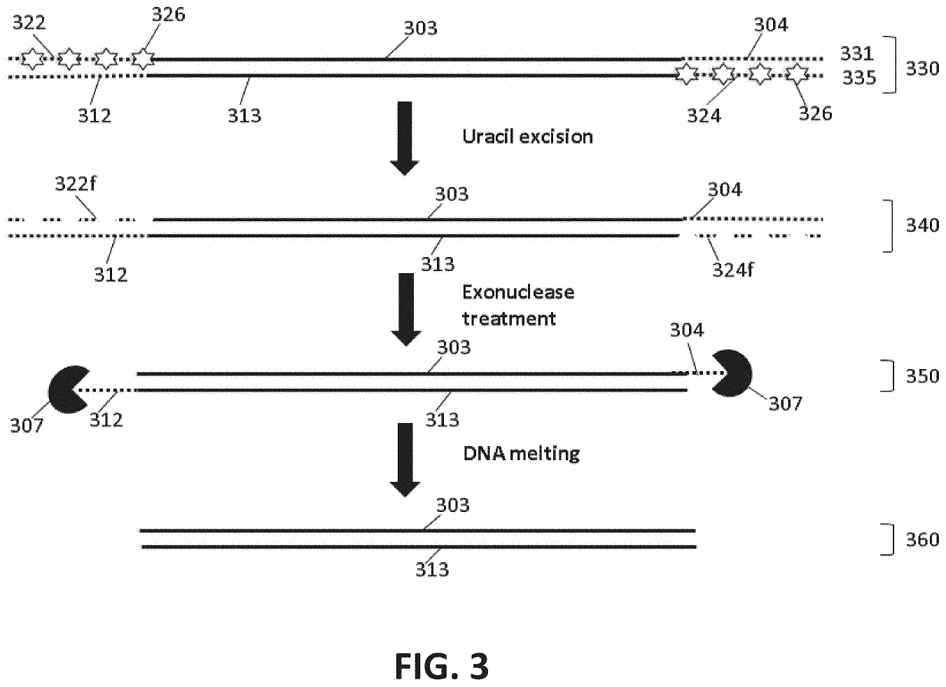

In another process for the removal of adapter nucleic acid sequence, a plurality of nucleobases that are not canonical DNA nucleobases are removed from a dsDNA molecule by DNA repair enzymes, exemplified in FIG. 3. In this illustration, a starting dsDNA molecule 330 is, optionally, a product of an amplification reaction that introduced the uracil nucleobases to the molecule. A first strand 331 of dsDNA molecule 330 comprises in 5′ to 3′ order: a first adapter sequence 322 comprising a plurality of uracil nucleobases 326, a target nucleic acid 303, and a second adapter 304. The second strand 335 of dsDNA molecule 330 comprises in 5′ to 3′ order: a third adapter sequence 324 comprising a plurality of uracil nucleobases 326, a sequence complementary to target nucleic acid 313, and a fourth adapter 312. For the process depicted in FIG. 3, the first, second, third and fourth adapter sequences are selected for removal in order to produce a final product consisting of target nucleic acid 303 and the sequence complementary to target nucleic acid 313. In a first step for removing adapter sequences, dsDNA molecule 330 is treated with one or more DNA repair enzymes to excise the plurality of uracil nucleobases 326 from: nucleic acid sequence 322, generating a nucleic acid sequence 322f comprising a plurality of gaps corresponding to previous base locations of the uracil nucleobases; nucleic acid sequence 324, generating a nucleic acid sequence 324f comprising a plurality of gaps corresponding to previous base locations of the uracil nucleobases. As a non-limiting example, the DNA repair enzymes have glycosylase and endonuclease activities. The resulting dsDNA molecule having gaps 340 of the uracil excision step is combined with a polymerase 307 at a melting temperature sufficient to remove remaining sequences 322f, 324f, 304 and 312 to generate dsDNA without non-target sequence 360, comprising the target nucleic acid 303 and the sequence complementary to target nucleic acid 313.

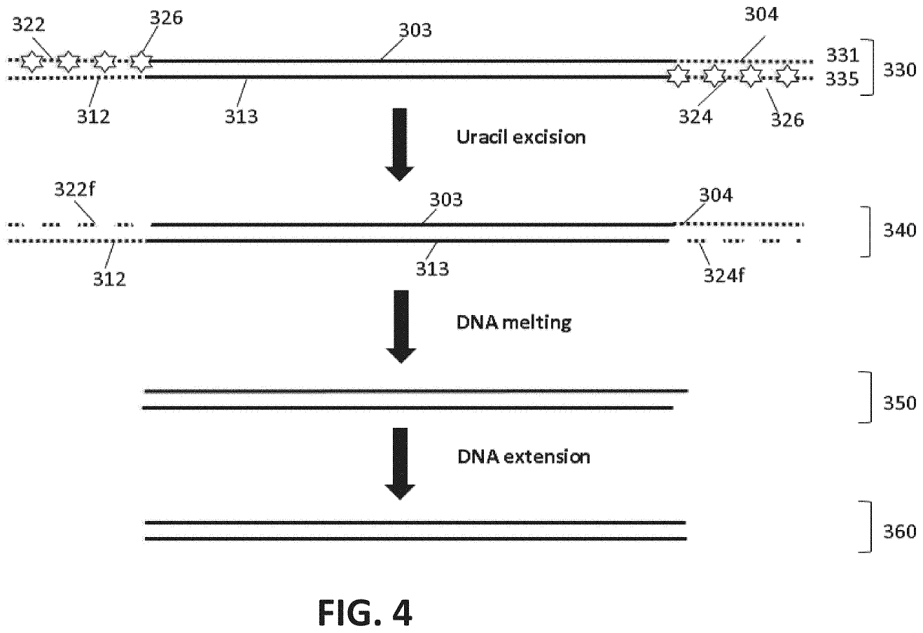

FIG. 4 illustrates a process for the removal of a non-target nucleic acid sequence from the dsDNA molecule 330 of FIG. 3, wherein dsDNA 330 is an amplicon product, for example, from an unpurified or “crude” amplification reaction. The dsDNA molecule 330 is treated with DNA repair enzymes as described in FIG. 3, and nucleobases that are not canonical DNA nucleobases (e.g., uracil) are excised. Following uracil excision, the dsDNA molecule having gaps 340 is subjected to a melting temperature, such as a temperature above 72° C. for about 15 minutes, to remove adapter sequences 322f, 324f, 304, 312 and generate dsDNA having overhangs 350. The dsDNA having overhangs 350 is cooled to a temperature below the melting temperature in a reaction mixture comprising dNTPs in an extension reaction to repair ends of the dsDNA having overhangs 350 and generate dsDNA without non-target sequence 360.

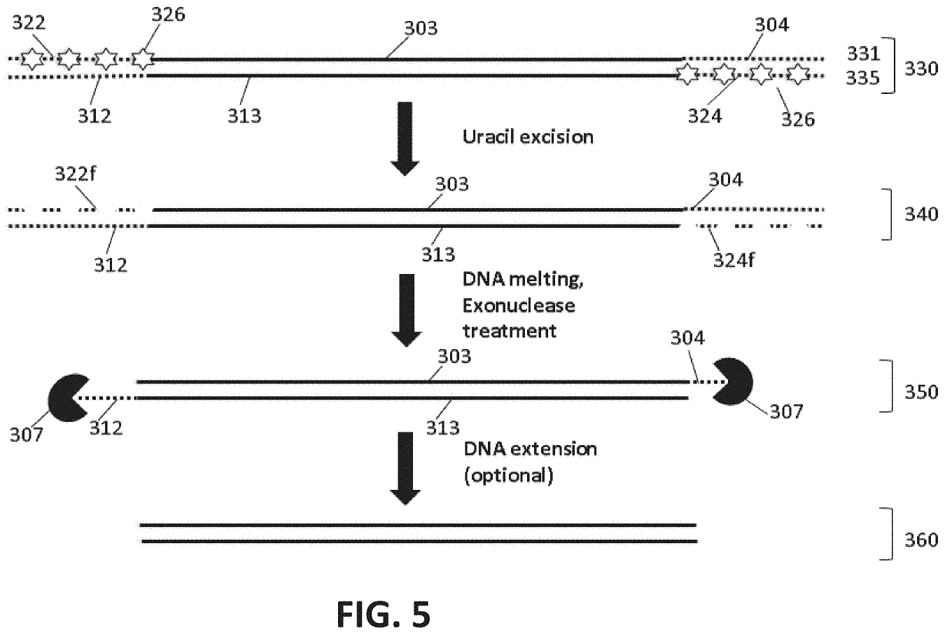

Another process for the removal of a non-target nucleic acid sequence from a dsDNA molecule 330 of FIG. 3 includes treatment of the dsDNA molecule DNA repair enzymes at an elevated temperature (see FIG. 5). Following excision of nucleobases that are not canonical DNA nucleobases (e.g., uracil bases), the dsDNA molecule having gaps 340 is treated with an enzyme having polymerase activity 307 at a melting temperature, such as a temperature above 72° C., to remove adapter sequence 322f, 324f, 304, 312 and generate dsDNA having overhangs 350. The dsDNA having overhangs 350 is cooled to a temperature below the melting temperature and optionally treated with dNTPs as to generate dsDNA without non-target sequence 360.

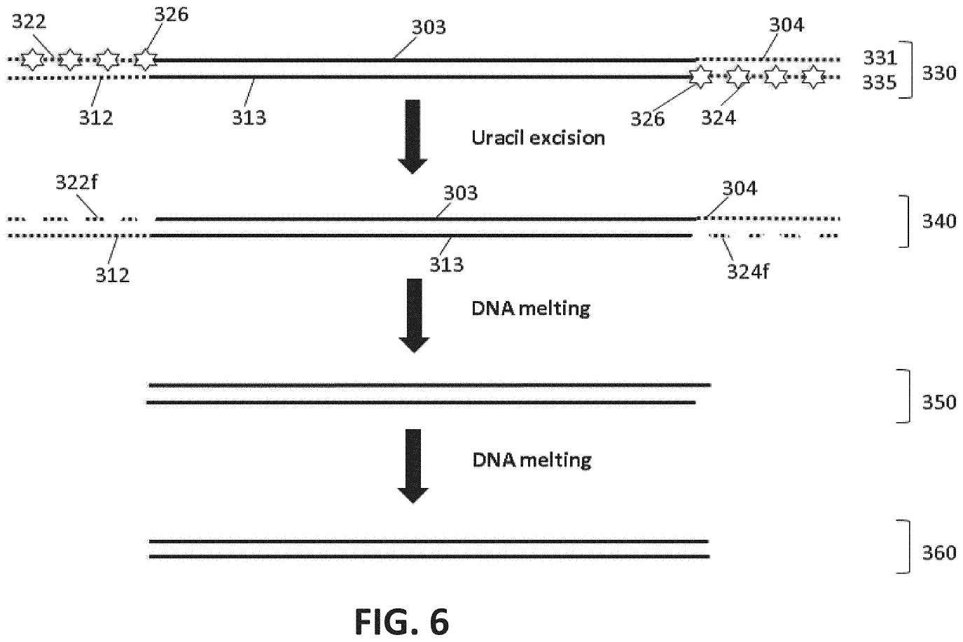

Another process for the removal of a non-target nucleic acid sequence from a dsDNA molecule includes multiple melting steps after excision of nucleobases that are not canonical DNA nucleobases (see FIG. 6). A dsDNA molecule 330 is treated with DNA repair enzymes, as described in FIG. 3, and the nucleobases that is not a canonical DNA nucleobases (e.g., uracil) are excised. Following excision, the dsDNA molecule having gaps 340 is subjected to a first melting temperature, such as a temperature above 90° C. for a short period of time (e.g., less than about 1 min), to melt nucleic acid sequences 322f and 324f The heat treated dsDNA molecule results in portions removed to form a dsDNA having overhangs 350, and is further incubated at a second melting temperature sufficient to remove remaining nucleic acids (304 and 312), for example, at a temperature between about 70° C. and about 80° C. for at least 5 minutes, generate dsDNA without non-target sequence 360.

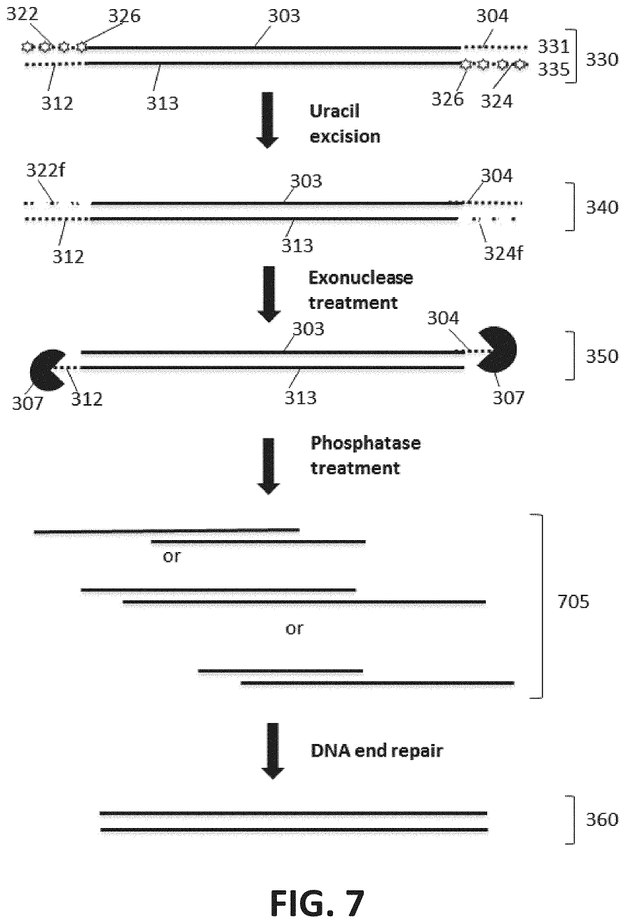

Another process for the removal of a non-target nucleic acid sequence from a dsDNA molecule 330 includes treatment with a phosphatase (see FIG. 7). A dsDNA molecule 330 is treated with DNA repair enzymes, as described in FIG. 3, nucleobases that are not canonical DNA nucleobases (e.g., uracil bases) are excised. Following excision of the nucleobases that are not canonical DNA nucleobases, the resulting excision product (dsDNA molecule having gaps 340) is treated with an enzyme having exonuclease activity 307, such as exonuclease III, to hydrolyze excess nucleic acids 304 and 312. The dsDNA is further treated with a phosphatase, such as shrimp alkaline phosphatase (SAP), to dephosphorylate excess nucleobases and generate phosphatase treatment product dsDNA 705. If dNTPs are present in the reaction mixture during phosphatase treatment, phosphatase treatment product dsDNA 705 is extended in a repair reaction to generate dsDNA without non-target sequence 360. Alternatively, dNTPs are supplied to the reaction mixture to repair the ends of phosphatase treatment product dsDNA 705 to generate dsDNA without non-target sequence 360. In some cases, dNTPs are present in the reaction mixture from an amplification reaction that occurs during production of dsDNA without non-target sequence 360.

In some aspects, methods described herein for the select removal of non-target sequence from a double-stranded nucleic acid comprising one or more modified bases result in double-stranded nucleic acid product comprising a desired target nucleic acid sequence with few or no bases remaining from the non-target sequence. In some cases, the product comprises fewer than 50, 45, 40, 35, 30, 25, 20, 15, 10, 9, 8, 7, 6, 5, 4, 3, 2, or 1 bases not part of a target nucleic acid sequence. In some cases, methods described herein result in a product comprising all or essentially all of the bases present the target nucleic acid sequence. In some cases, the product comprises fewer than 50, 45, 40, 35, 30, 25, 20, 15, 10, 9, 8, 7, 6, 5, 4, 3, 2, or 1 bases missing from the target nucleic acid sequence. In some cases, the number of extra bases in a product and/or the number of missing bases in a product is dependent on the identity of the target nucleic acid sequence, the length of the target nucleic acid sequence, the identity of enzymes used in the methods, reaction temperatures, unwanted sequence (e.g., primer sequence, adapter sequence, extension sequence), modified base identity, the number of modified bases in the starting material, or any combination thereof.

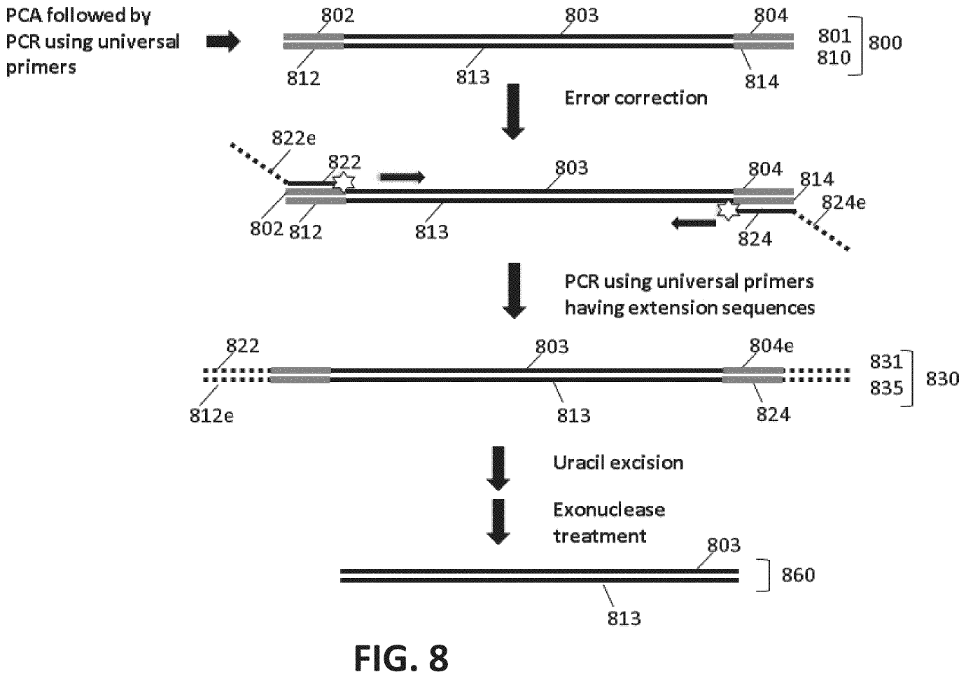

FIG. 8 illustrates a process for amplifying a target nucleic acid using a pair of universal primers comprising extension sequences. A PCA amplicon 800 comprising a first nucleic acid strand 801 and a second nucleic acid strand 810 is prepared. PCA amplicon 800 is a product of sequential PCA and PCR reactions, wherein the PCA and/or PCR reaction provide the PCA amplicon with a set of adapter sequences. The first nucleic acid strand 801 comprises in 5′ to 3′ order: a first adapter 802, a target nucleic acid 803, and a second adapter 804. The second nucleic acid strand 810 comprises in 5′ to 3′ order: a nucleic acid sequence reverse complementary to the second adapter or “second adapter complement” 814, a sequence reverse complementary to the target nucleic acid sequence or “target nucleic acid complement” 813, and a nucleic acid sequence reverse complementary to the first adapter sequence or “first adapter complement” 812. The PCA amplicon 800 is combined with a forward universal primer 822 and a reverse universal primer 824, each primer comprising a modified base (denoted by a star in FIG. 8) at the 3′ terminal end. The forward universal primer 822 comprises a first extension sequence 822e and a sequence that hybridizes to first adapter complement 812. The reverse universal primer 824 comprises a second extension sequence 824e and a sequence that hybridizes to second adapter 804. The PCA amplicon 800 is amplified, for example, by polymerase chain reaction (PCR), in an amplification reaction mixture comprising: PCA amplicon 800, the forward universal primer 822, the reverse universal primer 824, a polymerase compatible with uracil such as KAPA HiFi HotStart Uracil, dNTPs, and a buffer comprising divalent cations (e.g., MgCl2). The PCA amplicon 800 is amplified to generate amplicon 830. Amplicon 830 comprises a first amplicon strand 831 and a second amplicon strand 835. The first amplicon strand 831 comprises in 5′ to 3′ order: the forward universal primer sequence 822, the first target nucleic acid sequence 803, and an extended second adapter sequence 804e, wherein the extended second adapter sequence 804e comprises the second adapter sequence 804 and a sequence complementary to the second extension sequence 824e. The second amplicon strand 835 comprises in 5′ to 3′ order: the reverse universal primer 824, the target nucleic acid complement 813, and an extended complementary first adapter sequence 812e, wherein the extended complementary first adapter sequence 812e comprises the complementary first adapter sequence 812 and a sequence complementary to the first extension sequence 822e. PCR Amplicon 830 thus comprises (i) a first amplicon strand 831 comprising two extension sequences and a modified base from the forward universal primer 822 and (ii) a second amplicon strand 835 comprising two extension sequences and a modified base from the reverse universal primer 824. The extension sequences are removed from amplicon 830 using excision methods as described in any of FIGS. 3-7.

Universal Primers

Provided herein are primers comprising one or more features that facilitate the removal of primer sequence from an amplification product. Such features include modified bases, and nucleobases that are not canonical DNA nucleobases that participate in non-canonical base pairing during nucleic acid amplification. In many cases, the terms “modified base” and “non-canonical base” are used interchangeably herein to describe a nucleobase which is not a cytosine, guanine, adenine or thymine. In order for a primer to base pair with an adapter, primers having modified bases have at least about 50%, 60%, 70%, 80%, 90%, or 95% of its bases involved in canonical A-T or G-C base pairing with an adapter. Exemplary base pairs include: homopurine pair, heteropurine pair, homopyrimidine pair, heteropyrimidine pair, and purine-pyrimidine pair, wherein a purine includes a modified purine and a pyrimidine includes a modified pyrimidine.

For an amplification reaction having a plurality of target nucleic acids of varying sequence, a universal primer binding sequence may be shared among the plurality of target nucleic acids in the amplification reaction. Primers described herein are inclusive of universal primers which hybridize to this shared universal binding sequence to amplify the plurality of target nucleic acids.

Nucleobases that are not canonical DNA nucleobases in primers described herein include, without limitation, uracil, 3-meA (3-methyladenine), hypoxanthine, 8-oxoG (7,8-dihydro-8-oxoguanine), FapyG, FapyA, Tg (thymine glycol), hoU (hydroxyuracil), hmU (hydroxymethyluracil), fU (formyluracil), hoC (hydroxycytosine), fC (formylcytosine), 5-meC (5-methylcytosine), 6-meG (O6-methylguanine), 7-meG (N7-methylguanine), EC (ethenocytosine), 5-caC (5-carboxylcytosine), 2-hA, EA (ethenoadenine), 5-fU (5-fluorouracil), 3-meG (3-methylguanine), and isodialuric acid. Modified bases of primers described herein include, without limitation, oxidized bases, alkylated bases, deaminated bases, pyrimidine derivatives, purine derivatives, ring-fragmented bases, and methylated bases. Non-limiting examples of primers having nucleobases that are not canonical DNA nucleobases or modified base are listed in Tables 2, 6, 10, 19, 22 and 27. As shown in these exemplary primer sequences, often a modified base is a 3′ terminal base of the primer. Further apparent from these examples is that a primer having a modified base is inclusive of one or more modified bases, and as such, the disclosure provides primers having a plurality of modified bases. For example, a primer has 1, 2, 3, 4, 5, 6, 7, 8, 9, or 10 modified bases.

The arrangement of modified bases within a primer often facilitates removal of the primer after a nucleic acid amplification reaction. For instance, modified or non-canonical bases are spaced throughout a primer sequence so that two adjacent modified or non-canonical bases are separated by about 2-10, 3-8, or 4-7 non-modified or canonical bases. In some cases, a modified base is located at least 1, 2, 3, 4, 5, 6, 7, 8, 9, 10 or more bases from another modified base in a primer sequence. However, this spacing is not always required and some primers have adjacent modified bases. In some cases, a primer having a length of 10-60 bases has 1-10 modified bases.

A primer provided herein is not limited in size and includes oligonucleic acids having any length suitable for selective hybridization to a primer binding site comprising its complementary sequence. For instance, a primer has a length less than about 60, 40, 30 or 20 bases, or a length of about 12 to 50, 10-60, 10-50, or 10-40 bases. An adapter sequence comprising a primer binding site generally has a length less than about 100, 80, 60, 50, or 40 bases, or about 10-100 or 20-80 bases. In many cases, the number of primer bases is dependent on the composition of bases in the primer such as the percentage of GC content in the primer and identity and number of modified bases in the primer. As a non-limiting example, a primer has a GC content between about 40% and 60%.

To facilitate hybridization to a primer binding site, primers described herein sometimes do not comprise, or comprise two or fewer secondary structures produced by intermolecular or intramolecular interactions. Primer secondary structures include hairpins, self-dimers and cross-dimers. Another way to facilitate hybridization to a specific binding site is to design a primer with a minimum length of identical consecutive bases, for example, fewer than 6, 5, 4, or 3 consecutive identical bases.

Further provided herein are primers comprising a sequence that hybridizes to a primer binding site on a template nucleic acid during amplification, and an extension sequence. In some cases, the extension sequence is less than about 100, 90, 80, 70, 60, 50, 40, 30, 20, or 10 bases in length.

In various aspects, primers described herein comprise a uracil nucleobase as a modified base. This uracil nucleobase is one that is capable of base-pairing with a guanine nucleobase, adenine, or derivative thereof. Uridines prepared in a primer disclosed herein include, without limitation, uridines which have undergone oxidation, nitration, halogenation, and/or alkylation. Non-limiting examples of uridines include dihydrouridine, 2-thiouridine, 4-thiouridine, pseudouridine, and uridine-5-oxyacetic acid, 2-thiouridine, 5-methyluridine, pseudouridine, 5-methyluridine 5′-triphosphate (m5U), 5-idouridine 5′-triphosphate (15U), 4-thiouridine 5′-triphosphate (S4U), 5-bromouridine 5′-triphosphate (BrSU), 2′-methyl-2′-deoxyuridine 5′-triphosphate (U2′m), 2′-amino-2′-deoxyuridine 5′-triphosphate (U2′NH2), 2′-azido-2′-deoxyuridine 5′-triphosphate (U2′N3) and 2′-fluoro-2′-deoxyuridine 5′-triphosphate (U2′F). Uridines also include those comprising a base modification and/or a sugar modification.

Primers described herein sometimes have a modified backbone. This includes the backbone connecting a modified base within the primer. In some cases, the backbone connecting the modified base is protected by a phosphorothioate linkage to minimize exonuclease digestion. In some cases, a primer is part of a peptide nucleic acid (PNA), which is a synthetic DNA or RNA analog where a peptide-like backbone replaces the sugar-phosphate backbone of DNA or RNA, respectively. In some cases, a primer comprises an unnatural backbone, including without limitation, phosphorothioates, chiral phosphorothioates, phosphorodithioates, phosphotriesters, aminoalkylphosphotriesters, C1-C10 phosphonates, 3′-alkylene phosphonates, chiral phosphonates, phosphinates, phosphoramidates, 3′-amino phosphoramidates, aminoalkylphosphoramidates, thionophosphoramidates, thionoalkylphosphonates, thionoalkylphosphotriesters, and boranophosphates.

Primers may be synthesized by any methods known in the art or commercially available, for example, by using a commercial synthesizer (e.g., AKTA Oligopilot; GE Healthcare Life Sciences; Pittsburgh, Pa.) or commercial supplier (e.g., Integrated DNA Technologies; Coralville, Iowa). Methods useful for primer synthesis include solid-phase synthesis and solution synthesis. In some cases, primers are assembled using PCA. Primer synthesis may include subsequent error correction. In some instances, primers described herein are biotinylated during or after synthesis. For example, biotinylated universal primers for amplifying a target nucleic acid sequence are useful for subsequent error processing methods.

Target Nucleic Acids

Various methods described herein involve the amplification of a target nucleic acid using a primer, which optionally comprises a modified base. Target nucleic acids may be generated by PCA of de novo synthesized precursor nucleic acids. In some cases, the length of a target nucleic acid is at least about 50, 80, 100, 200, or 300 bases. In some cases, a target nucleic acid has a length up to about 1000, 2000, 3000, 4000 bases or more.

When a template nucleic acid is amplified with a primer having a modified base that is not present in the template, the resulting amplicon will often have properties that differ from the template nucleic acid due to the incorporation of a modified base. For example, an amplicon comprising a modified base has a melting temperature about 1-10° C. higher or lower than its template nucleic acid lacking the modified base.

In some amplification methods where a plurality of target nucleic acids are to be amplified in a single batch, the plurality of target nucleic acids are appended with a shared adapter sequence that comprises a primer binding site for amplification. In order to add these adapter sequences to each of the plurality of target nucleic acids, often a portion of an adapter sequence differs from another adapter sequence based on the target nucleic acid sequence. For example, an adapter sequence comprises a shared universal primer binding sequence and a sequence specific for a target nucleic acid. In some cases, adapter sequences differ from each other by at least about 1, 2, 5, 10, 15, 20, 25, 30, 35, 40, 45, 50, or more, nucleobases. In some cases, at least about 1, 2, 3, 4, 5, 10, 20, 25, 30, 50, 100, or more adapters are used in a multiplexed fashion. In some instances, each differing adapter sequence efficiently binds to a universal primer at a different temperature.

An exemplary method for preparing a nucleic acid sequence comprising a target nucleic acid sequence and optionally one or more adapter sequences involves polymerase chain assembly (PCA) or polymerase chain reaction assembly (PCR assembly). PCR assembly uses polymerase-mediated chain extension in combination with at least two oligonucleic acids having complementary ends which can anneal such that at least one of the polynucleotides has a free 3′-hydroxyl capable of polynucleotide chain elongation by a polymerase (e.g., a thermostable polymerase such as Taq polymerase, VENT™ polymerase (New England Biolabs), KOD (Novagen) and the like). Overlapping oligonucleic acids may be mixed in a standard PCR reaction containing dNTPs, a polymerase, and buffer. The overlapping ends of the oligonucleic acids, upon annealing, create regions of double-stranded nucleic acid sequences that serve as primers for the elongation by polymerase in a PCR reaction. Products of the elongation reaction serve as substrates for formation of a longer double-strand nucleic acid sequences, eventually resulting in the synthesis of full-length target sequence. The PCR conditions may be optimized to increase the yield of the target long DNA sequence.

In some cases, a target nucleic acid sequence comprises an extension sequence at one or more of its ends. In some cases, the extension sequence is a primer or a component of a primer. In some cases, a primer comprises an extension sequence upstream of a nucleic acid sequence complementary to an adapter sequence, a target sequence, or both an adapter and target sequence. In some cases, dsDNA molecule comprises a plurality of dsDNA molecules, wherein one or more extension sequences of one dsDNA molecule is the same as one or more extension sequences of another dsDNA molecule. In some cases, a dsDNA molecule comprises a plurality of dsDNA molecules, wherein one or more extension sequences of one dsDNA molecule is different than one or more extension sequences of another dsDNA molecule. Exemplary extension sequence lengths include 1-100, 1-90, 1-80, 1-70, 1-60, 1-50, 1-40 and 1-30 bases. In some cases, the bases of an extension sequence have similar properties to a primer as described elsewhere herein, for example, similar melting temperatures, similar annealing temperatures, and/or similar GC content.

During some methods provided herein, a double-stranded target nucleic acid is dissociated into single strands at a melting temperature. This melting temperature includes temperatures of at least about 45° C., 50° C., 55° C., 60° C., 70° C., or between about 45° C. and 70° C. In some cases, melting occurs following treatment of a double-stranded target nucleic acid amplicon with one or more DNA repair enzymes to remove a modified base, which results in an amplicon having a fragmented sequence (“fragmented amplicon”). The melting is performed at a temperature sufficient to melt nucleic acid sequences to remove the fragmented sequence. In some cases, the fragmented amplicon is heated for a short period of time, for example, less than 5 minutes, 2 minutes, 1 minute, 40 seconds, 30 seconds, 20 seconds, 10 seconds, or 5 seconds. In some cases, the fragmented amplicon is further subjected to temperatures sufficient to remove nucleic acids complementary to the removed fragmented nucleic acids (overhangs). Exemplary temperatures for overhang removal include those about or greater than about 60° C., 62° C., 64° C., 66° C., 68° C., 70° C., 72° C., 74° C., 76° C., 78° C., 80° C., 60° C. or 90° C., or about 60-80° C. or 70-80° C.

Enzymatic Reactions

Enzymes are disclosed herein for multiple purposes. For example, enzymatic reactions disclosed herein include amplification reactions, nicking reactions, and cleavage reactions. In some cases, a dsDNA molecule having unwanted nucleic acid sequence is treated with one or more DNA repair enzymes, a phosphatase, an enzyme having exonuclease activity, a polymerase, heat treated, or any combination thereof. Treatment of the dsDNA molecule results in a dsDNA product lacking the unwanted nucleic acid sequence.

Polymerase Reactions

Various amplification reactions and primer removal methods described herein employ a polymerase. For amplification reactions having a primer with a modified base, a polymerase selected for the amplification reaction is capable of recognizing the modified base. For example, a modified base is a uracil and a DNA polymerase is a uracil compatible DNA polymerase. Non-limiting examples of uracil compatible DNA polymerases include Pfu polymerase, Pfu Turbo Cx and KAPA HiFi Uracil+. Uracil compatible DNA polymerases also include polymerases and derivative thereof (e.g., mutants, chimeras) from archaea such as Pyrococcus furiosus.

Non-limiting examples of polymerases for use in methods described herein include: DNA polymerase, RNA polymerase, reverse transcriptase, primase and transferase. Polymerases include naturally-occurring polymerases and any modified variations thereof, including, but not limited to, mutants, recombinants, fusions, genetic modifications, chemical modifications, synthetics, and analogs. Naturally-occurring polymerases and modified variations thereof are not limited to polymerases which retain the ability to catalyze a polymerization reaction. In some instances, the naturally-occurring and/or modified variations thereof retain the ability to catalyze a polymerization reaction. Mutant polymerases include polymerases wherein one or more amino acids are replaced with other amino acids (naturally or non-naturally occurring), and insertions or deletions of one or more amino acids. In some instances, a polymerase is a fusion protein comprising at least two regions linked, e.g., a polymerase is T7 DNA polymerase, which comprises a nucleic acid polymerizing domain and a thioredoxin binding domain, wherein thioredoxin binding enhances the processivity of the polymerase.

In some methods, a single DNA polymerase or a plurality of DNA polymerases are used throughout a reaction. In some cases, the same DNA polymerase or set of DNA polymerases are used at different stages of the present methods or the DNA polymerases are varied or additional polymerase added during various steps. In some cases, a polymerase is a thermostable DNA polymerase, for example stable at temperatures greater than about 70° C. In some instances, a DNA polymerase is stable at temperature necessary to melt fragmented DNA from the DNA product, for example at temperatures above about 70° C.

Some methods described herein utilize a high fidelity DNA polymerase, wherein the DNA polymerase has strong 3′ exonuclease activity. For example, to remove 3′ overhangs comprising primer sequence or complement primer sequence from an amplification product. Non-limiting examples of high fidelity DNA polymerases include KAPA HiFi polymerase, KAPA HiFi Uracil+polymerase, Phusion®, PrimeSTAR® DNA polymerase, Platinum® Pfx DNA polymerase, Pfx50™ DNA polymerase, Elongase® DNA polymerase, HotStar HiFidelity Polymerase, Deep Vent™ DNA polymerase and Q5® High Fidelity DNA polymerase. In some instances, a high fidelity DNA polymerase has a decreased error rate as compared to a non-high fidelity DNA polymerase. In some cases, a high fidelity DNA polymerase comprises a processivity-enhancing domain. In some cases, a high fidelity DNA polymerase does not comprise an accessory protein domain such as a processivity-enhancing domain. In some instances, a high fidelity DNA polymerase is useful on targets having up to about 75%, up to about 78%, up to about 80%, up to about 82%, up to about 84%, up to about 85%, or up to about 90% GC content.

DNA Repair Enzymatic Reactions

Various primer removal methods described herein involve excision of a modified base from the primer in an amplification product. As a non-limiting example, the modified base is a uracil nucleobase. In some such cases, excision of a modified base is achieved with a DNA repair enzyme. A DNA repair enzyme includes a DNA glycosylase that catalyzes a first step in base excision by removing a base from a nucleic acid while leaving the backbone of the nucleic acid intact, generating an apurinic or apyrimidinic site, or AP site. This removal is accomplished by flipping the base out of a double stranded nucleic acid followed by cleavage of the N-glycosidic bond. In some cases, excision of a modified base occurs when a glycosylase removes the modified base from a nucleic acid by N-glycosylase activity. The resulting apurinic/apyrimidinic (AP) site is then incised by the AP lyase activity of bifunctional glycosylase via β-elimination of the 3′ phosphodiester bond.

DNA repair enzymes are primarily used at a temperature optimal for enzymatic activity, for example, a temperature of 25-80° C., 25-70° C., 25-60° C., 25-50° C., or 25-40° C. In some cases, reactions involving a DNA repair enzyme occur for at least about 5, 10, 15, 30, 45, 60, 75, 90, 120, 180, or 240 minutes or about 30-150 minutes. In some instances, a DNA repair enzyme is inactivated after use, for example, by an inhibitor or heat.

Glycosylase and/or DNA repair enzymes may recognize a uracil or a base pair comprising uracil, for example U:G and/or U:A. Nucleic acid base substrates recognized by a glycosylase include, without limitation, uracil, 3-meA (3-methyladenine), hypoxanthine, 8-oxoG, FapyG, FapyA, Tg (thymine glycol), hoU (hydroxyuracil), hmU (hydroxymethyluracil), fU (formyluracil), hoC (hydroxycytosine), fC (formylcytosine), oxidized base, alkylated base, deaminated base, methylated base, and any modified nucleobase provided herein or known in the art. In some instances, the glycosylase and/or DNA repair enzyme recognizes oxidized bases such as 2,6-diamino-4-hydroxy-5-formamidopyrimidine (FapyG) and 8-oxoguanine (8-oxo). Glycosylases and/or DNA repair enzymes which recognize oxidized bases include, without limitation, OGG1 (8-oxoG DNA glycosylase 1) or E. coli Fpg (recognizes 8-oxoG:C pair), MYH (MutY homolog DNA glycosylase) or E. coli MutY (recognizes 8-oxoG:A), NEIL1, NEIL2 and NEIL3. In some instances, the glycosylase and/or DNA repair enzyme recognizes methylated bases such as 3-methyladenine. An example of a glycosylase that recognizes methylated bases is E. coli AlkA or 3-methyladenine DNA glycosylase II, Mag1 and MPG (methylpurine glycosylase). Additional non-limiting examples of glycosylases include SMUG1 (single-strand specific monofunctional uracil DNA glycosylase 1), TDG (thymine DNA glycosylase), MBD4 (methyl-binding domain glycosylase 4), and NTHL1 (endonuclease III-like 1). Exemplary DNA glycosylases include, without limitation, uracil DNA glycosylases (UDGs), helix-hairpin-helix (HhH) glycosylases, 3-methyl-purine glycosylase (MPG) and endonuclease VIII-like (NEIL) glycosylases. Helix-hairpin-helix (HhH) glycosylases include, without limitation, Nth (homologs of the E. coli EndoIII protein), OggI (8-oxoG DNA glycosylase I), MutY/Mig (A/G-mismatch-specific adenine glycosylase), AlkA (alkyladenine-DNA glycosylase), MpgII (N-methylpurine-DNA glycosylase II), and OggII (8-oxoG DNA glycosylase II). Exemplary 3-methyl-puring glycosylases (MPGs) substances include, in non-limiting examples, alkylated bases including 3-meA, 7-meG, 3-meG and ethylated bases. Endonuclease VIII-like glycosylase substrates include, without limitation, oxidized pyrimidines (e.g., Tg, 5-hC, FaPyA, PaPyG), 5-hU and 8-oxoG.

Exemplary uracil DNA glycosylases (UDGs) include, without limitation, thermophilic uracil DNA glycosylases, uracil-N glycosylases (UNGs), mismatch-specific uracil DNA glycosylases (MUGs) and single-strand specific monofunctional uracil DNA glycosylases (SMUGs). In non-limiting examples, UNGs include UNG1 isoforms and UNG2 isoforms. In non-limiting examples, MUGs include thymidine DNA glycosylase (TDG). A UDG may be active against uracil in ssDNA and dsDNA. For further descriptions of UDGs see Aravind L, Koonin EV (2000) The alpha/beta fold uracil DNA glycosylases: a common origin with diverse fates. Genome Biol 1, wherein the described UDGs are incorporated herein by reference.

Certain enzymes described herein, such as an endonuclease, exonuclease, glycosylase, and/or DNA repair enzyme recognize a mismatch base-pair that is not an A-T or G-C base pair. One or both the bases in the mismatch base-pair are then removed by the enzyme. For example, the TDG enzyme is capable of excising thymine from G:T mismatches. Endonucleases are often employed to nick DNA in the region of mismatches or damaged DNA, including but not limited to T7 Endonuclease I, E. coli Endonuclease V, T4 Endonuclease VII, mung bean nuclease, Cel-1 endonuclease, E. coli Endonuclease IV and UVDE. Cel-1 endonuclease from celery and similar enzymes, typically plant enzymes, exhibit properties that detect a variety of errors in double stranded nucleic acids. For example, such enzymes can detect polynucleotide loops and insertions, detect mismatches in base pairing, recognize sequence differences in polynucleotide strands between about 100 bp and 3 kb in length and recognize such mutations in a target polynucleotide sequence without substantial adverse effects of flanking DNA sequences.

In some cases, a modified base is released from a dsDNA molecule by a DNA glycosylase resulting in an abasic site. This abasic site (AP site) is further processed by an endonuclease which cleaves the phosphate backbone at the abasic site. Endonucleases include AP endonucleases such as class I and class II AP endonucleases, which incise DNA at the phosphate groups 3′ and 5′ to the baseless site leaving 3′ OH and 5′ phosphate termini. In some cases, an endonuclease is a class III or class IV AP endonuclease which cleaves DNA at the phosphate groups 3′ and 5′ to the baseless site to generate 3′ phosphate and 5′ OH.

AP endonucleases are grouped into families based on sequence similarity and structure, for example, AP endonuclease family 1 or AP endonuclease family 2. Examples of AP endonuclease family 1 members include, without limitation, E. coli exonuclease III, S. pneumoniae and B. subtilis exonuclease A, mammalian AP endonuclease 1 (API), Drosophila recombination repair protein 1, Arabidopsis thaliana apurinic endonuclease-redox protein, Dictyostelium DNA-(apurinic or apyrimidinic site) lyase, enzymes comprising one or more domains thereof, and enzymes having at least 75% sequence identity to one or more domains or regions thereof. Examples of AP endonuclease family 2 members include, without limitation, bacterial endonuclease IV, fungal and Caenorhabditis elegans apurinic endonuclease APN1, Dictyostelium endonuclease 4 homolog, Archaeal probable endonuclease 4 homologs, mimivirus putative endonuclease 4, enzymes comprising one or more domains thereof, and enzymes having at least 75% sequence identity to one or more domains or regions thereof. Exemplary, endonucleases include endonucleases derived from both Prokaryotes (e.g., endonuclease IV, RecBCD endonuclease, T7 endonuclease, endonuclease II) and Eukaryotes (e.g., Neurospora endonuclease, S1 endonuclease, P1 endonuclease, Mung bean nuclease I, Ustilago nuclease). In some cases, an endonuclease functions as both a glycosylase and an AP-lyase. In some cases, the endonuclease is endonuclease VIII. In some instances, the endonuclease is S1 endonuclease. In some cases, the endonuclease is endonuclease III. In some cases, the endonuclease is endonuclease IV. In some instances, an endonuclease is a protein comprising an endonuclease domain having endonuclease activity that cleaves a phosphodiester bond.

In some primer removal methods provided herein, a modified base of the primer is removed with a DNA excision repair enzyme and endonuclease or lyase, wherein the endonuclease or lyase activity is optionally from an excision repair enzyme or a region of the excision repair enzyme. Excision repair enzymes include, without limitation, Methyl Purine DNA Glycosylase (recognizes methylated bases), 8-Oxo-GuanineGlycosylase 1 (recognizes 8-oxoG:C pairs and has lyase activity), Endonuclease Three Homolog 1 (recognizes T-glycol, C-glycol, and formamidopyrimidine and has lyase activity), inosine, hypoxanthine-DNA glycosylase; 5-Methylcytosine, 5-Methylcytosine DNA glycosylase; Formamidopyrimidine-DNA-glycosylase (excision of oxidized residue from DNA: hydrolysis of the N-glycosidic bond (DNA glycosylase), and beta-elimination (AP-lyase reaction)). In some cases, the DNA excision repair enzyme is uracil DNA glycosylase. DNA excision repair enzymes include also include, without limitation, Aag (catalyzes excision of 3-methyladenine, 3-methylguanine, 7-methylguanine, hypoxanthine, 1,N6-ethenoadenine), endonuclease III (catalyzes excision of cis- and trans-thymine glycol, 5,6-dihydrothymine, 5,6-dihydroxydihydrothymine, 5-hydroxy-5-methylhydantoin, 6-hydroxy-5,6-dihydropyrimidines, 5-hydroxycytosine and 5-hydroxyuracil, 5-hydroxy-6-hydrothymine, 5,6-dihydrouracil, 5-hydroxy-6-hydrouracil, AP sites, uracil glycol, methyltartronylurea, alloxan), endonuclease V (cleaves AP sites on dsDNA and ssDNA), Fpg (catalyzes excision of 8-oxoguanine, 5-hydroxycytosine, 5-hydroxyuracil, aflatoxin-bound imidazole ring-opened guanine, imidazole ring-opened N-2-aminofluorene-C8-guanine, open ring forms of 7-methylguanine), and Mug (catalyzes the removal of uracil in U:G mismatches in double-stranded oligonucleic acids, excision of 3, N4-ethenocytosine (eC) in eC:G mismatches in double-, or single-stranded oligonucleic acids). Non-limiting DNA excision repair enzymes are listed in Curr Protoc Mol Biol. 2008 October; Chapter 3:Unit3.9, herein incorporated by reference. DNA excision repair enzymes, such as endonucleases, may be selected to excise a specific modified base. As an example, endonuclease V, T. maritima is a 3′-endonuclease which initiates the removal of deaminated bases such as uracil, hypoxanthine, and xanthine. In some cases, a DNA excision repair enzyme having endonuclease activity functions to remove a modified or non-canonical base from a strand of a dsDNA molecule without the use of an enzyme having glycosylase activity.

DNA repair enzymes may comprise glycosylase activity, lyase activity, endonuclease activity, or any combination thereof. In some methods, one or more DNA excision repair enzymes are used, for example one or more glycosylases or a combination of one or more glycosylases and one or more endonucleases. As an example, Fpg (formamidopyrimidine [fapy]-DNA glycosylase), also known as 8-oxoguanine DNA glycosylase, acts both as a N-glycosylase and an AP-lyase. The N-glycosylase activity releases a modified base (e.g., 8-oxoguanine, 8-oxoadenine, fapy-guanine, methy-fapy-guanine, fapy-adenine, aflatoxin Bi-fapy-guanine, 5-hydroxy-cytosine, 5-hydroxy-uracil) from dsDNA, generating an abasic site. The lyase activity then cleaves both 3′ and 5′ to the abasic site thereby removing the abasic site and leaving a 1 base gap or nick. Additional enzymes which comprise more than enzymatic activities include, without limitation, endonuclease III (Nth) protein from E. coli (N-glycosylase and AP-lyase) and Tma endonuclease III (N-glycosylase and AP-lyase). For a list of DNA repair enzymes having lyase activity, see the New England BioLabs® Inc. catalog.

Exonuclease Reactions

In some primer removal methods, one or more modified bases are excised from a dsDNA molecule which is subsequently treated with an enzyme comprising exonuclease activity. In some cases, the exonuclease comprises 3′ DNA polymerase activity. Exonucleases include those enzymes in the following groups: exonuclease I, exonuclease II, exonuclease III, exonuclease IV, exonuclease V, exonuclease VI, exonuclease VII, and exonuclease VIII. In some instances, an exonuclease has AP endonuclease activity. In some cases, the exonuclease is any enzyme comprising one or more domains or amino acid regions suitable for cleaving nucleotides from either 5′ or 3′ end or both ends, of a nucleic acid chain. Exonucleases include wild-type exonucleases and derivatives, chimeras, and/or mutants thereof. Mutant exonucleases include enzymes comprising one or more mutations, insertions, deletions or any combination thereof within the amino acid or nucleic acid sequence of an exonuclease.

Exonucleases are often used at a temperature optimal for enzymatic activity, for example, a temperature of 25-80° C., 25-70° C., 25-60° C., 25-50° C., or 25-40° C. In some cases, reactions involving an exonuclease occur for at least about 1, 5, 10, 15, 30, 45, 60, 75, 90, 120, 180, or 240 minutes or about 30-150 minutes. In some instances, exonuclease is inactivated after use, for example, by an inhibitor or heat.

Nuclease Reactions

Some methods for removing a select sequence from a nucleic acid involve treating the nucleic acid with an enzyme comprising nuclease activity, such as S1 nuclease. In some cases, the nuclease is combined with a nucleic acid comprising a nick between a primer sequence and a target sequence, wherein the nuclease removes the primer sequence. Nuclease reactions often occur at a temperature optimal for enzymatic activity, for example, a temperature of 25-80° C., 25-70° C., 25-60° C., 25-50° C., or 25-40° C. In some cases, nuclease reactions occur for at least about 1, 5, 10, 15, 30, 45, 60, 75, 90, 120, 180, or 240 minutes or about 30-150 minutes. In some instances, a nuclease is inactivated after use, for example, by an inhibitor or heat.

Phosphatase Reactions

Some reactions described herein utilize a phosphatase, such as shrimp alkaline phosphatase, to hydrolyze dNTPs in the reaction mixture. In some cases, the reaction mixture comprises dNTPs from a previous amplification step producing an amplicon comprising an incorporated primer having a modified base. In some cases, a phosphatase is combined in a reaction mixture with the amplicon after removal of the modified base, for example, by using one or more DNA repair enzymes. In some cases, a phosphatase is combined in a reaction mixture comprising an exonuclease and the amplicon after removal of the modified base. In some cases, a phosphatase is combined with an amplicon after treatment with a DNA repair enzyme and exonuclease. Phosphatase reactions often occur at a temperature optimal for enzymatic activity, for example, a temperature of 25-80° C., 25-70° C., 25-60° C., 25-50° C., or 25-40° C. In some cases, phosphatase reactions occur for at least about 1, 5, 10, 15, 30, 45, 60, 75, 90, 120, 180, or 240 minutes or about 30-150 minutes. In some instances, a phosphatase is inactivated after use, for example, by an inhibitor or heat.

End Repair Reactions

In some cases, methods for removing modified nucleic acid sequences and their complementary sequences from a dsDNA molecule results in a pool of dsDNA molecules having first and second nucleic acid strands with different 5′ and/or 3′ ends resulting from digestion with one or more enzymes, for example, an enzyme having exonuclease activity. These digested nucleic acid strands are end-repaired by providing the dsDNA molecules with dNTPs and optionally a polymerase at a temperature suitable for annealing. In some cases, this end-repair reaction occurs in about 5-120 minutes, or about 10, 15, 20, 25, 30, 45, or 60 minutes. In some cases, the end-repair reaction occurs at a temperature of about 50-80° C., 60-80° C., 70-80° C. or about 72° C. In some cases, end-repaired dsDNA comprise a first target nucleic acid sequence and a second target nucleic acid sequence having the same sequence as their dsDNA starting material, for example, an amplicon, prior to treatment to remove modified nucleic acids. In some cases, end-repair occurs by the addition of dNTPs at about 68° C., 69° C., 70° C., 71° C., 72° C., 73° C., 74° C., 75° C., or 76° C.

The temperatures and time durations for one or more steps of a method provided herein are sometimes dependent on the sequence identity, length, composition, GC content, etc. of a primer sequence, target nucleic acid sequence, adapter sequence, and any combination thereof. In some cases, the temperatures and time durations for one or more steps of a method provided herein comprising one or more enzymes are dependent on the identity of the one or more enzymes.

Various methods described herein comprise one or more modular steps which may be combined with another step or methods described herein. Such methods include synthesis of target nucleic acids optionally comprising an adapter sequences and/or modified base, amplification of a target nucleic acid with a primer comprising a modified base, removal of non-target nucleic acid sequences such as primer sequences, adapter sequences, and extension sequences, and methods for end-repair.

Amplification Reactions

Various methods and systems described herein employ nucleic acid amplification reactions performed by any method known in the art that results in production of a plurality of copies of a template nucleic acid. Although certain embodiments herein exemplify nucleic acid amplification by a polymerase chain reaction (PCR), the methods are not limited to this type of reaction and should not be construed as limiting. Accordingly, reference herein to PCR is extended to any amplification reaction described herein or known in the art. Non-limiting methods of nucleic acid amplification include ligase chain reaction, oligonucleic acid ligations assay, hybridization assay, amplified fragment length polymorphism, allele-specific PCR, Alu PCR, asymmetric PCR, Helicase-dependent isothermal DNA amplification, hot start PCR, inverse PCR, in situ PCR, intersequence-specific PCR, digital PCR, linear-after-the-exponential-PCR, long PCR, nested PCR, real-time PCR, duplex PCR, multiplex PCR, quantitative PCR, quantitative fluorescent PCR (QF-PCR), multiplex fluorescent PCR (MF-PCR), restriction fragment length polymorphism PCR (PCR-RFLP), PCK-RFLPIRT-PCR-IRFLP, polonony PCR, rolling circle amplification (RCA), bridge PCR, picotiter PCR, emulsion PCR, single cell PCR, transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleic acid-primed PCR (DOP-PCR), amplification of a single stranded nucleic acid using a single oligonucleic acid primer, nucleic acid sequence based amplification (NASBA), Q-beta-replicase method, 3SR, Transcription Mediated Amplification (TMA), and Strand Displacement Amplification (SDA). In some instances, amplification methods are solid-phase amplification, polony amplification, colony amplification, emulsion PCR, bead RCA, surface RCA, surface SDA, etc., as will be recognized by one of skill in the art. In some instances, a target nucleic acid, adapter sequence, and/or primer is immobilized to a surface during a nucleic acid amplification reaction. Surfaces include those which are planer, microparticles, and nanoparticles. In some instances, amplification is performed in a solution, without restraint of a reaction component tethered to a surface.

For some nucleic acid amplification reactions, one or more bases in a primer is labeled with a distinguishing and/or detectable tag. The tag may be distinguishable by fluorescence, Raman spectrum, charge, mass, refractive index, luminescence, length, or any other measurable property. The labeled primer is sometimes useful for monitoring an amplification reaction described herein, for example, in a real-time PCR.

Amplification reaction mixtures often include, but are not limited to, enzymes such as a polymerase, dNTPs, template nucleic acid molecules comprising a target nucleic acid sequence, primer nucleic acids, salts, buffers, small molecules, co-factors, metals, ions, chelating agents and salts. Ions include divalent catalytic ions such as Mg2+ or Mn2+, Co2+, and Ba2+; non-catalytic ions such as Ca2+, St2+, Zn2+, Cu2+, Co2+, Fe2+, and Ni2+; as well as and non-covalent metal ions. Salts include NaCl, KCl, K-acetate, NH4-acetate, K-glutamate, NH4Cl, and (NH4)2S04. Buffers include Tris, Tricine, HEPES, MOPS, ACES, MES, phosphate-based buffers, and acetate-based buffers. Chelating agents include EDTA and EGTA. In some cases, an amplification reaction mixture comprises a cross-linking reagent.

Provided herein are methods for amplifying a plurality of non-identical molecules using a single primer pair population, wherein each primer comprises a 3′ modified base. In many amplification methods, the 3′ modified base is a uracil nucleobase. The method comprises tagging the plurality of non-identical molecules with identical primer binding sites in an adapter sequence. An amplification reaction is performed using the primer pair population, wherein the primers anneal to their cognate identical primer binding site. The amplification reaction products comprise the incorporated primers in place of the identical primer binding sites. The incorporated primers are removed using any method or combination of methods described herein for removing nucleic acid sequences comprising one or more modified bases.

DNA Libraries

Provided herein are libraries comprising a plurality of dsDNA molecules having different sequences generated using a nucleic acid amplification reaction described herein. As an example, a plurality of template nucleic acids having different target sequences each comprise shared forward and reverse primer binding sequences within an adapter appended to each of the different target sequences. A pair of universal primers specific for the shared forward and reverse primer binding sequences is used to amplify the plurality of template nucleic acids. The universal primers comprise a modified base such as a uracil to facilitate removal of universal primer sequence from the amplification products. The universal primer sequences are removed in a process involving excision of the modified base as detailed elsewhere herein to generate a library of DNA molecules comprising different target sequences. Further provided herein are libraries comprising any intermediate product of a nucleic acid amplification reaction described herein. For example, each dsDNA molecule in a library comprises a shared universal primer binding sequence comprising one or more modified bases. In addition, a library of DNA molecules may also comprise one or more enzymes used during any reaction described herein, such as a DNA repair enzyme, endonuclease, glycosylase, exonuclease, and/or polymerase. Libraries provided herein also include DNA molecules purified from any reaction described herein.

Libraries provided herein may comprise a large number of different target nucleic acid sequences. For example, a library comprises more than 100, 200, 300, 400, 500, 600, 750, 1000, 15000, 20000, 30000, 40000, 50000, 60000, 75000, 100000, 200000, 300000, 400000, 500000, 600000, 750000, 1000000, 2000000, 3000000, 4000000, 5000000, or more different target nucleic acids. The different nucleic acids may be related to predetermined/preselected sequences. In some instances, the library comprises nucleic acids that are over 500 bp, 600 bp, 700 bp, 800 bp, 900 bp, 1000 bp, 1250 bp, 1500 bp, 1750 bp, 2000 bp, 2500 bp, 3000 bp, 4000 bp, 5000 bp, 6000 bp, 7000 bp, 8000 bp, 9000 bp, 10 kb, 20 kb, 30 kb, 40 kb, 50 kb, 60 kb, 80 kb, 90 kb, or 100 kb in length. It is understood that a library may comprise of a plurality of different subsections, such as 2, 3, 4, 5, 6, 7, 8, 9, 10 subsections or more, that are governed by different construct sizes.

Applications

Nucleic acids prepared and amplified using the methods and compositions disclosed herein may be used in any application including, by way of example, probes for hybridization methods such as gene expression analysis, genotyping by hybridization (competitive hybridization and heteroduplex analysis), sequencing by hybridization, probes for Southern blot analysis (labeled primers), probes for array (either microarray or filter array) hybridization, “padlock” probes usable with energy transfer dyes to detect hybridization in genotyping or expression assays, and other types of probes. The nucleic acids prepared in accordance with the this disclosure may also be used in enzyme-based reactions such as polymerase chain reaction (PCR), as primers for PCR, templates for PCR, allele-specific PCR (genotyping/haplotyping) techniques, real-time PCR, quantitative PCR, reverse transcriptase PCR, and other PCR techniques. The nucleic acids may be used for various ligation techniques, including ligation-based genotyping, oligo ligation assays (OLA), ligation-based amplification, ligation of adapter sequences for cloning experiments, Sanger dideoxy sequencing (primers, labeled primers), high throughput sequencing (using electrophoretic separation or other separation method), primer extensions, mini-sequencings, and single base extensions (SBE).

The nucleic acids produced in accordance with this disclosure may be used in mutagenesis studies, (introducing a mutation into a known sequence with an oligo), reverse transcription (making a cDNA copy of an RNA transcript), gene synthesis, introduction of restriction sites (a form of mutagenesis), protein-DNA binding studies, and like experiments.

Methods provided herein produce DNA products lacking extraneous nucleobases such as adapter sequences or primers from an amplification reaction. In some cases, these DNA products are used in recombinant DNA technologies such as cloning. In some cases, the DNA products are used in vivo in a cell. For example, the DNA products are expressed in a cell such as an eukaryotic cell, a prokaryotic cell, or a viral cell.

Provided herein are systems for performing one or more methods or one or more steps of a method described herein. In some instances, a system comprises components and reagents necessary to perform nucleic acid hybridization between a primer comprising a modified nucleobase and a nucleic acid comprising a target sequence. This nucleic acid hybridization occurs during an amplification reaction, wherein the nucleic acid comprising the target sequence is amplified, generating amplicons comprising the primer sequence and modified base. In some instances, a system comprises components and reagents necessary to remove the modified base from the amplicon. In some instances, a system comprises components and reagents necessary to remove unwanted fragmented nucleic acid sequence from the amplicon after removal of the modified base.

De Novo Nucleic Acid Synthesis

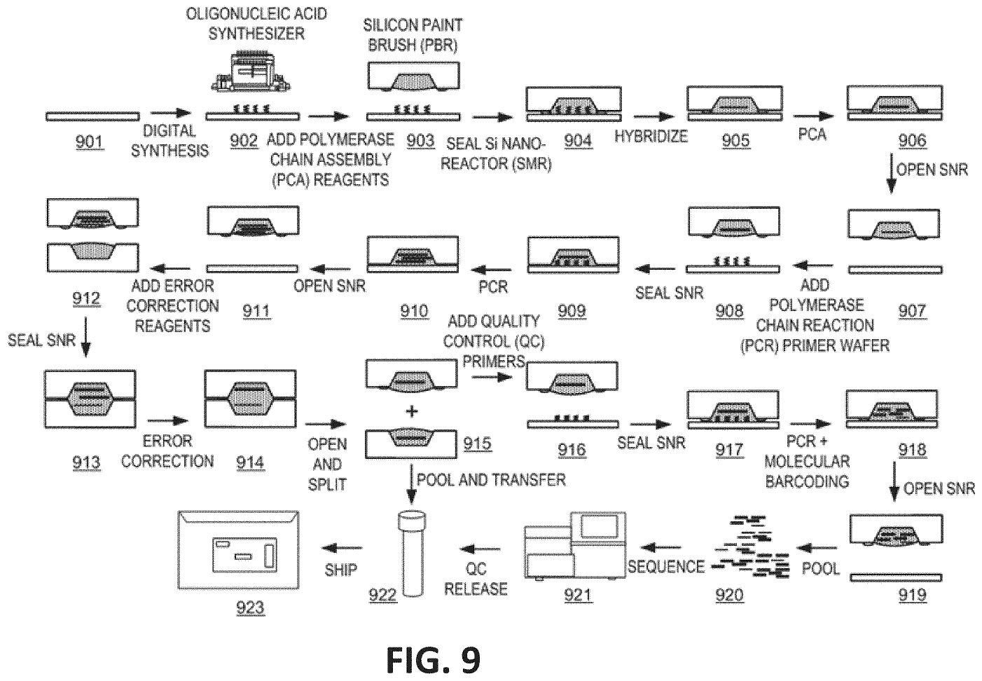

Referring to FIG. 9, an exemplary process workflow is depicted for for synthesis of large nucleic acids, where the large nucleic acids are amplified using one or more methods described herein. The workflow can be divided generally into phases: (1) de novo synthesis of a single stranded oligonucleic acid library, (2) joining oligonucleic acids to form larger fragments, (3) error correction, (4) quality control, and (5) shipment. Prior to de novo synthesis, an intended nucleic acid sequence or group of nucleic acid sequences is preselected. For example, a group of genes is preselected for generation.

Once large oligonucleic acids for generation are selected, a predetermined library of oligonucleic acids is designed for de novo synthesis. Various suitable methods are known for generating high density oligonucleic acid arrays. In the workflow example, a substrate surface layer 901 is provided. In the example, chemistry of the surface is altered in order to improve the oligonucleic acid synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry.

In situ preparation of oligonucleic acid arrays is generated on a solid support and utilizes single nucleotide extension process to extend multiple oligomers in parallel. A device, such as an inkjet printer, is designed to release reagents in a step wise fashion such that multiple oligonucleic acids extend, in parallel, one residue at a time to generate oligomers with a predetermined nucleic acid sequence 902. In some cases, oligonucleic acids are cleaved from the surface at this stage. Cleavage may include gas cleavage, e.g., with ammonia or methylamine.

The generated oligonucleic acid libraries are placed in a reaction chamber. In this exemplary workflow, the reaction chamber (also referred to as “nanoreactor”) is a silicon coated well, containing PCR reagents and lowered onto the oligonucleic acid library 903. Prior to or after the sealing 904 of the oligonucleic acids, a reagent is added to release the oligonucleic acids from the substrate. In the exemplary workflow, the oligonucleic acids are released subsequent to sealing of the nanoreactor 905. Once released, fragments of single stranded oligonucleic acids hybridize in order to span an entire long range sequence of DNA. Partial hybridization 905 is possible because each synthesized oligonucleic acid is designed to have a small portion overlapping with at least one other oligonucleic acid in the pool.

After hybridization, a PCA reaction is commenced. During the polymerase cycles, the oligonucleic acids anneal to complementary fragments and gaps are filled in by a polymerase. Each cycle increases the length of various fragments randomly depending on which oligonucleic acids find each other. Complementarity amongst the fragments allows for forming a complete large span of double stranded DNA 906.

After PCA is complete, the nanoreactor is separated from the substrate 907 and positioned for interaction with a substrate (also referred to as “wafer”) having primers for PCR 908. After sealing, the nanoreactor is subject to PCR 909 and the larger nucleic acids are amplified. After PCR 910, the nanochamber is opened 911, error correction reagents are added 912, the chamber is sealed 913 and an error correction reaction occurs to remove mismatched base pairs and/or strands with poor complementarity from the double stranded PCR amplification products 914. The nanoreactor is opened and separated 915. Error corrected product is next subject to additional processing steps, such as PCR and molecular bar coding, and then packaged 922 for shipment 923. The additional process step is sometimes PCR, which is performed using a pair of universal primers having one or more modified nucleobases as described herein. The universal primer sequences are then removed from the amplification products using any primer removal method described herein.

In some cases, quality control measures are taken. After error correction, PCR amplification with universal primers, and universal primer removal, quality control steps include interaction with another wafer having sequencing primers for amplification of the error corrected product 916, sealing the wafer to a chamber containing error corrected amplification product 917, and performing an additional round of amplification 918. The nanoreactor is opened 919 and the products are pooled 920 and sequenced 921. After an acceptable quality control determination is made, the packaged product 922 is approved for shipment 923.

Computers and Software

In various instances, methods and systems of the described herein further comprise software programs on computer systems and uses thereof. Accordingly, computerized control for the optimization of methods described herein (e.g., amplification, enzyme treatment), including the supply of reagents and control of reaction conditions, as well as design of primers for use according to the methods, are within the bounds of this disclosure.

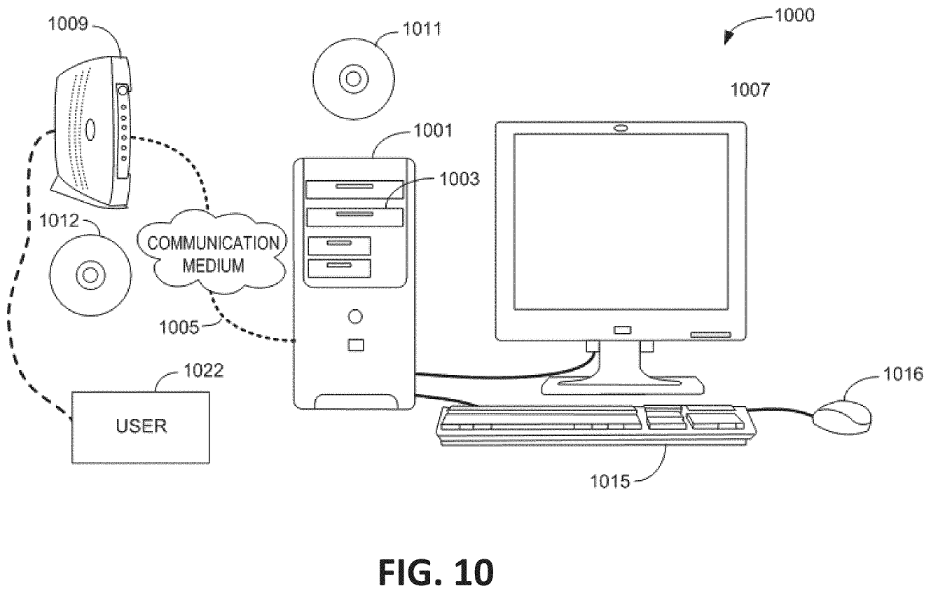

FIG. 10 illustrates an exemplary of a computer system 1000 useful for implementing one or more methods described herein. The computer system 1000 is a logical apparatus that can read instructions from media 1011 and/or a network port 1005, which can optionally be connected to server 1009 having fixed media 1012. The system can include a CPU 1001, disk drives 10, optional input devices such as keyboard 1015 and/or mouse 1016 and optional monitor 1007. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 1022 as illustrated in FIG. 10.

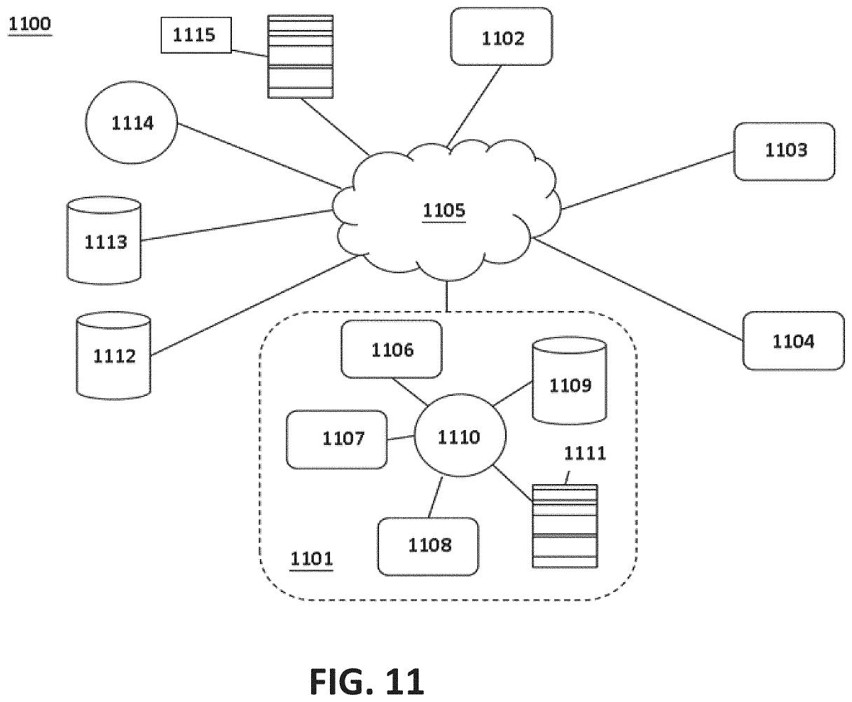

FIG. 11 provides an exemplary system 1100 for implementing one or more methods described herein. The system 1100 may be adapted to interface with various entities or systems associated with such entities, such as, for example, a third party provider or a system associated with a third party provider, a user network provider or a system associated with a user network provider, or a user or a system associated with a user. The systems associated with entities can include computer systems.

The system 1100 can include a computer system 1101 that is in communication with a first entity 1102 (e.g., a third party provider), a second entity 1103 (e.g., a user network provider) and a third entity 1104 (e.g., a user). The system 1100 can interface with an entity with the aid of a network 1105. The network 1105 may include the Internet, an intranet and the extranet. For example, the network 1105 can be the Internet or an intranet that is operatively coupled to the Internet. In some contexts, the network 1105 can be referred to as the “cloud.” In some cases, multiple networks can be used for interfacing with each entity or for interfacing with different entities.

The computer system (“system”) 1101 includes a memory location 1106, a communications interface 1107, a display interface 1108 and, in some cases, a data storage unit 1109, which are all operatively coupled to a processor 1110, such as a central processing unit (CPU) or a plurality of CPU's for parallel processing. The system 1101 may include one or more servers, such as, for example, data or database servers, file servers, web servers, or application servers. The system 1101 can have software that is configured to operate on various operating systems, such as Linux-based operating systems, Windows-based operating systems, or any other operating system described herein. The operating system can reside on a memory location of the system 1101. In some cases, the operating system can be provided by cloud computing.

The memory location 1106 may include one or more of flash memory, cache and a hard disk. In some situations, the memory location 1106 is read-only memory (ROM) or random-access memory (RAM), to name a few examples. The data storage unit 1109 can include one or more hard disks, memory and/or cache for data transfer and storage. The data storage unit 1109 can include one or more databases, such as, for example, document-oriented database (e.g., MongoDB), relational databases (e.g., Microsoft® SQL Server, mySQL™, Oracle®), non-relational databases, object or object-oriented databases, entity-relationship model databases, associative databases, and XML databases. In some cases, the system 1101 further includes a data warehouse for storing information, such as user information. In some examples, the data warehouse resides on a computer system remote from the system 1101. In further examples, one or more components of the system 1101 can reside on a computer system remote from the system 1101. In some cases, remote components may be added in addition to components residing on the system 1101. For example, data storage units 1112 and 1113, a processor 1114, or a server 1115 can be in communication with the computer system 1101 over the network 1105.

The communications interface 1107 can include a network interface for allowing the system 1101 to interact with the network 1105, which may include an intranet, including other systems and subsystems, and the Internet, including the World Wide Web. In some cases, the communications interface 1107 includes interfaces for enabling the system 1100 to interact with multiple networks. The system 1101 may include one or more communication interfaces or ports (COM PORTS), or one or more input/output (I/O) modules, such as an I/O interface.

In some situations, the communications interface 1107 functions with the system 1101 to wirelessly interface with the network 1105. In such a case, the communications interface 1107 includes a wireless interface (e.g., 2G, 3G, 4G, long term evolution (LTE), WiFi, Bluetooth) that brings the system 1101 in wireless communication with a wireless access point that is in communication with the network 1105.

The communications interface 1107 may be configured to allow the system 1101 to collect information from various sources (e.g., user network information from user network providers, or third party information from third party providers). For example, the system 1101 can be programmed or otherwise configured to access user network information.

The computer system of the user 1104 can include, for example, a personal computer (PC), a terminal, a server, a slate or tablet PC, a smart phone, a netbook, a personal digital assistant, or systems and devices with optional computer network connectivity. For example, the system 1104 can be a user terminal comprising a display and an input device such as a keyboard, a pointing device, a touch screen, a microphone to capture voice or other sound input, or a video camera or other sensor to capture motion or visual input. In another example, the system 1104 can comprise a memory location (e.g., a hard disk) and a processor in addition to the display and the input device. In some cases, the system 1104 may also comprise a data storage unit. The computer system 1104 can comprise an operating system, such as, for example, a server operating system, a personal computer operating system, or a mobile or smart phone operating system. In some implementations, the operating system is provided by cloud computing.

Information may be communicated between various components of the system 1100 over the network 1105 to facilitate processing and/or storage. As an example, software and algorithms can be configured to be processed locally by the user (e.g., by the processor on the user system 1104), remotely (e.g., by the processor 1114), remotely via a cloud server (e.g., server 1115), remotely by the system 1101 (e.g., by the processor 1110), or a combination thereof. In some cases, when user terminals are used, software and algorithms may be configured to only be processed remotely. In some implementations, software and associated data of the system 1100 can be centrally hosted on the cloud (e.g., on the computer system 1101, the data storage units 1112 and 1113, the processor 1114, the server 1115, or a combination thereof) and accessed by users using a thin client via a web browser (e.g., via the network 1105). In some examples, a client-server architecture is provided that may require installation of software on the user system 1104. In some examples, different user access levels may be provided. For example, individual users may be able to access the system 1100 at any or at limited levels of the system hierarchy.

In some examples, the user interface is a web-based user interface (also “web interface” herein) that is configured (e.g., programmed) to be accessed using an Internet of a computer system of the user 1104. Multiple threads of execution can be used for parallel processing. In some instances, multiple processors or processors with multiple cores can be used, whether in a single computer system, in a cluster, or distributed across systems over a network comprising a plurality of computers, cell phones, and/or personal data assistant devices.

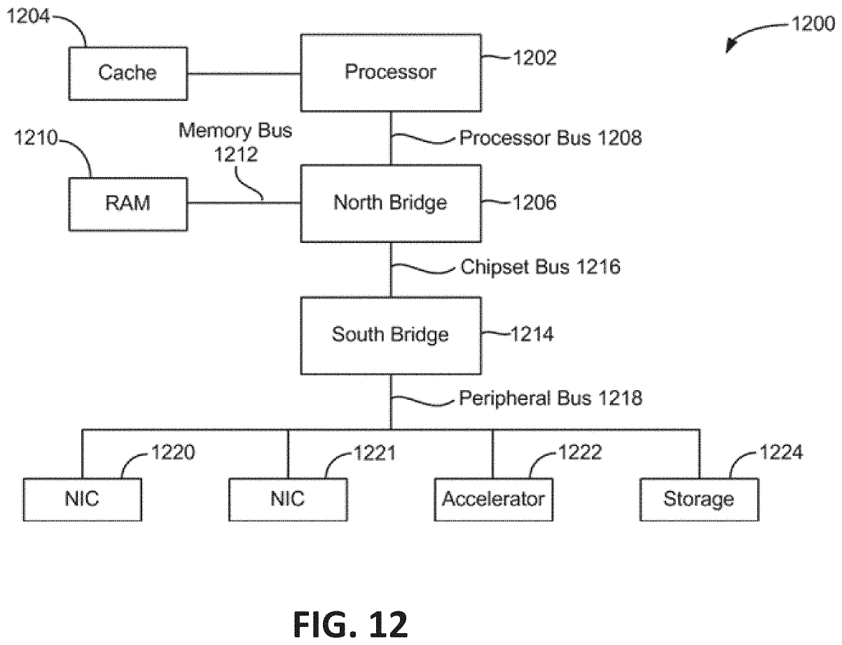

As illustrated in FIG. 12, a high speed cache 1201 can be connected to, or incorporated in, the processor 1202 to provide a high speed memory for instructions or data that have been recently, or are frequently, used by processor 1202. The processor 1202 is connected to a north bridge 1206 by a processor bus 1205. The north bridge 1206 is connected to random access memory (RAM) 1203 by a memory bus 1204 and manages access to the RAM 1203 by the processor 1202. The north bridge 1206 is also connected to a south bridge 1208 by a chipset bus 1207. The south bridge 1208 is, in turn, connected to a peripheral bus 1209. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 1209. In some architectures, the functionality of the north bridge can be incorporated into the processor instead of using a separate north bridge chip.

Software and data are stored in external storage 1213 and can be loaded into RAM 1203 and/or cache 1201 for use by the processor. The system 1200 includes an operating system for managing system resources; non-limiting examples of operating systems include: Linux, Windows™, MACOS™, BlackBerry OS™, iOS™, and other functionally-equivalent operating systems, as well as application software running on top of the operating system.

The above computer architectures and systems are examples only, and a wide variety of other computer, cell phone, and personal data assistant architectures and systems can be used in connection with exemplary arrangements described herein, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used, including random access memory, hard drives, flash memory, tape drives, disk arrays, Network Attached Storage (NAS) and other local or distributed data storage devices and systems.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: PCR Amplification of a Target Sequence Using Universal Primers Comprising One or More Modified Bases

A plurality of DNA duplexes comprising complementary nucleic acid strands were amplified by PCR. The first nucleic acid strand in each duplex comprised in 5′ to 3′ order: a first adapter sequence, a first target nucleic acid sequence, and a second adapter sequence. The second nucleic acid strand in each duplex comprised in 5′ to 3′ order: a nucleic acid sequence with a sequence reverse complement to the second adapter sequence (“complementary second adapter sequence”), a second target nucleic acid with a sequence reverse complement to the first target nucleic acid sequence, and a nucleic acid sequence with a sequence reverse complement to the first adapter sequence (“complementary first adapter sequence”). Nucleic acid sequences for the first strands of each DNA duplex are provided in Table 1, where the corresponding nucleic acid sequences for the second strands of each DNA duplex comprise a nucleic acid sequence reverse complementary to each first strand sequence.

TABLE 1

| | First | | Second | |
|---|---|---|---|---|
| Adaptor | adapter | | adapter | |
| name | sequence | First target sequence | sequence | First strand sequence |
| Uni5 | Uni5_1: | ATCTGAAGCGGAGTCC | Uni5_2: | GCCGTACTCTCAACTCACATATCTG |
| | GCCGTA | ACACAACAAGATCGGG | AGACCAG | AAGCGGAGTCCACACAACAAGATC |
| | CTCTCA | TTAATTGAATGCTCAGA | GCTACGA | GGGTTAATTGAATGCTCAGATGGT |
| | ACTCAC | TGGTGCGCCGTCTGTCC | TACAAC | GCGCCGTCTGTCCTCTCTCTTGTCC |
| | AT (SEQ | TCTCTCTTGTCCTCTAC | (SEQ ID | TCTACGGATCAGTAGCCAGAGATT |
| | ID NO: 5) | GGATCAGTAGCCAGAG | NO: 11) | GTTGCTACTAGGCTCCGAGAAGAG |
| | | ATTGTTGCTACTAGGCT | | ACTCGAAGCAGGATAGACCAGGCT |
| | | CCGAGAAGAGACTCGA | | ACGATACAAC (SEQ ID NO: 16) |
| | | AGCAGGAT (SEQ ID NO: | | |
| | | 10) | | |
| Uni6v21 | Uni6v21_1 | ATCTGAAGCGGAGTCC | Uni6v21_2 | GGACTTGTATGCTGACTGCTATCTG |
| | GGACT | ACACAACAAGATCGGG | AGACCGT | AAGCGGAGTCCACACAACAAGATC |
| | TGTATG | TTAATTGAATGCTCAGA | AGGGCAA | GGGTTAATTGAATGCTCAGATGGT |
| | CTGACT | TGGTGCGCCGTCTGTCC | TGATTC | GCGCCGTCTGTCCTCTCTCTTGTCC |
| | GCT | TCTCTCTTGTCCTCTAC | (SEQ ID | TCTACGGATCAGTAGCCAGAGATT |
| | (SEQ ID | GGATCAGTAGCCAGAG | NO: 12) | GTTGCTACTAGGCTCCGAGAAGAG |
| | NO: 6) | ATTGTTGCTACTAGGCT | | ACTCGAAGCAGGATAGACCGTAGG |
| | | CCGAGAAGAGACTCGA | | GCAATGATTC (SEQ ID NO: 17) |
| | | AGCAGGAT (SEQ ID NO: | | |
| | | 10) | | |
| Uni7 | Uni7_1: | ATCTGAAGCGGAGTCC | Uni7_2: | GACTCGTCAGCATCAAGACTATCTG |
| | GACTCG | ACACAACAAGATCGGG | ATGCTTG | AAGCGGAGTCCACACAACAAGATC |
| | TCAGCA | TTAATTGAATGCTCAGA | ATGAGTG | GGGTTAATTGAATGCTCAGATGGT |
| | TCAAGA | TGGTGCGCCGTCTGTCC | ACCTGG | GCGCCGTCTGTCCTCTCTCTTGTCC |
| | CT (SEQ | TCTCTCTTGTCCTCTAC | (SEQ ID | TCTACGGATCAGTAGCCAGAGATT |
| | ID NO: 7) | GGATCAGTAGCCAGAG | NO: 13) | GTTGCTACTAGGCTCCGAGAAGAG |
| | | ATTGTTGCTACTAGGCT | | ACTCGAAGCAGGATATGCTTGATG |
| | | CCGAGAAGAGACTCGA | | AGTGACCTGG (SEQ ID NO: 18) |
| | | AGCAGGAT (SEQ ID NO: | | |
| | | 10) | | |
| Uni8 | Uni8_1: | ATCTGAAGCGGAGTCC | Uni8_2: | GTTCTATCCAACTGCGGTCTATCTG |
| | GTTCTA | ACACAACAAGATCGGG | AGCAGGT | AAGCGGAGTCCACACAACAAGATC |
| | TCCAAC | TTAATTGAATGCTCAGA | AGTGATA | GGGTTAATTGAATGCTCAGATGGT |
| | TGCGGT | TGGTGCGCCGTCTGTCC | CTTGGC | GCGCCGTCTGTCCTCTCTCTTGTCC |
| | CT (SEQ | TCTCTCTTGTCCTCTAC | (SEQ ID | TCTACGGATCAGTAGCCAGAGATT |
| | ID NO: 8) | GGATCAGTAGCCAGAG | NO: 14) | GTTGCTACTAGGCTCCGAGAAGAG |
| | | ATTGTTGCTACTAGGCT | | ACTCGAAGCAGGATAGCAGGTAGT |
| | | CCGAGAAGAGACTCGA | | GATACTTGGC (SEQ ID NO: 19) |
| | | AGCAGGAT (SEQ ID NO: | | |
| | | 10) | | |
| Uni-GT | UniGT_1 | ATCTGAAGCGGAGTCC | UniGT_2: | GTTGGGTGGGTGGTGTGTGTATCTG |
| | GTTGGG | ACACAACAAGATCGGG | ACCACAC | AAGCGGAGTCCACACAACAAGATC |
| | TGGGTG | TTAATTGAATGCTCAGA | ACCACCC | GGGTTAATTGAATGCTCAGATGGT |
| | GTGTGT | TGGTGCGCCGTCTGTCC | ACCACA | GCGCCGTCTGTCCTCTCTCTTGTCC |
| | GT (SEQ | TCTCTCTTGTCCTCTAC | (SEQ ID | TCTACGGATCAGTAGCCAGAGATT |
| | ID NO: 9) | GGATCAGTAGCCAGAG | NO: 15) | GTTGCTACTAGGCTCCGAGAAGAG |
| | | ATTGTTGCTACTAGGCT | | ACTCGAAGCAGGATACCACACACC |
| | | CCGAGAAGAGACTCGA | | ACCCACCACA (SEQ ID NO: 20) |
| | | AGCAGGAT (SEQ ID NO: | | |
| | | 10) | | |

Table 1. Target nucleic acid sequences to be amplified using universal primers.

The DNA duplexes were amplified using a set of primers comprising or not comprising one or more uracil bases. The forward primers comprised a sequence from their respective first adapter sequence. The reverse primers comprised a sequence from their respective complementary second adapter sequence. Some primers comprised a uracil at their 3′ ends. Some primers comprised one or more internal uracils. Table 2 provides sets of primers corresponding to the adapter sequences of Table 1. An internal uracil is denoted by “ideoxyU”. A 3′ uracil is denoted by “3deoxyU”. Uracil bases protected at their 3′ end with a phosphorothioate bond are marked with an asterisk, “*”.

TABLE 2

| Primer | Target | SEQ ID | |
|---|---|---|---|
| Name | adapter | NO. | Sequence (5′ to 3′) |
| Uni5-0U_F | Uni5_1 | 5 | GCCGTACTCTCAACTCACAT |
| Uni5-1U_F | Uni5_1 | 21 | GCCGTACTCTCAACTCACA*/3deoxyU/ |
| Uni5-2U_F | Uni5_1 | 22 | GCCGTACTC/ideoxyU/CAACTCACA*/3deoxyU/ |
| Uni5-4U_F | Uni5_1 | 23 | GCCG/ideoxyU/ACTC/ideoxyU/CAAC/ideoxyU/CACA*/3deoxyU/ |
| UNI5-0U_R | Uni5_2 | 24 | GTTGTATCGTAGCCTGGTCT |
| UNI5-1U_R | Uni5_2 | 25 | GTTGTATCGTAGCCTGGTC*/3deoxyU/ |
| UNI5-2U_R | Uni5_2 | 26 | GTTGTATCG/ideoxyU/AGCCTGGTC*/3deoxyU/ |
| UNI5-4U_R | Uni5_2 | 27 | GTTG/ideoxyU/ATCG/ideoxyU/AGCC/ideoxyU/GGTC*/3deoxyU/ |
| Uni6v2-0U_F | Uni6v21_1 | 6 | GGACTTGTATGCTGACTGCT |
| Uni6v2-1U_F | Uni6v21_1 | 28 | GGACTTGTATGCTGACTGC*/3deoxyU/ |
| Uni6v2-2U_F | Uni6v21_1 | 29 | GGACTTGTA/ideoxyU/GCTGACTGC*/3deoxyU/ |
| Uni6v2-3U_F | Uni6v21_1 | 30 | GGACT/ideoxyU/GTATGC/ideoxyU/GACTGC*/3deoxyU/ |
| Uni6v2-5U_F | Uni6v21_1 | 31 | GGAC/ideoxyU/TGTA/ideoxyU/GC/ideoxyU/GAC/ideoxyU/ |
| | | | GC*/3deoxyU/ |
| Uni6v1-0U_R | Uni6v21_2 | 32 | GAATCATTGCCCTACGGTCT |
| Uni6v1-1U_R | Uni6v21_2 | 33 | GAATCATTGCCCTACGGTC*/3deoxyU/ |
| Uni6v1-3U_R | Uni6v21_2 | 34 | GAATCA/ideoxyU/TGCCC/ideoxyU/ACGGTC*/3deoxyU/ |
| Uni6v1-5U_R | Uni6v21_2 | 35 | GAA/ideoxyU/CAT/ideoxyU/GCCC/ideoxyU/ACGG/ideoxy |
| | | | U/C*/3deoxyU/ |
| Uni7-0U_F | Uni7_1 | 7 | GACTCGTCAGCATCAAGACT |
| Uni7-1U_F | Uni7_1 | 36 | GACTCGTCAGCATCAAGAC*/3deoxyU/ |
| Uni7-3U_F | Uni7_1 | 37 | GACTCG/ideoxyU/CAGCA/ideoxyU/CAAGAC*/3deoxyU/ |
| Uni7-0U_R | Uni7_2 | 38 | CCAGGTCACTCATCAAGCAT |
| Uni7-1U_R | Uni7_2 | 39 | CCAGGTCACTCATCAAGCA*/3deoxyU/ |
| Uni7-3U_R | Uni7_2 | 40 | CCAGG/ideoxyU/CACTCA/ideoxyU/CAAGCA*/3deoxyU/ |
| Uni8-0U_F | Uni8_1 | 8 | GTTCTATCCAACTGCGGTCT |
| Uni8-1U_F | Uni8_1 | 41 | GTTCTATCCAACTGCGGTC*/3deoxyU/ |
| Uni8-3U_F | Uni8_1 | 42 | GTTCTA/ideoxyU/CCAAC/ideoxyU/GCGGTC*/3deoxyU/ |
| Uni8-0U_R | Uni8_2 | 43 | GCCAAGTATCACTACCTGCT |
| Uni8-1U_R | Uni8_2 | 44 | GCCAAGTATCACTACCTGC*/3deoxyU/ |
| Uni8-3U_R | Uni8_2 | 45 | GCCAAG/ideoxyU/ATCAC/ideoxyU/ACCTGC*/3deoxyU/ |
| GT-0U_R | Uni-GT_2 | 46 | TGTGGTGGGTGGTGTGTGGT |
| GT-1U_R | Uni-GT_2 | 47 | TGTGGTGGGTGGTGTGTGG*/3deoxyU/ |
| GT-2U_R | Uni-GT_2 | 48 | TGTGGTGGG/ideoxyU/GGTGTGTGG*/3deoxyU/ |
| GT-3U_R | Uni-GT_2 | 49 | TGTGG/ideoxyU/GGGTGG/ideoxyU/GTGTGG*/3deoxyU/ |
| GT-4U_R | Uni-GT_2 | 50 | TGTGG/ideoxyU/GGG/ideoxyU/GGTG/ideoxyU/GTGG*/3deoxyU/ |
| GT-6U_R | Uni-GT_2 | 51 | TG/ideoxyU/GG/ideoxyU/GGG/ideoxyU/GG/ideoxyU/GTG/ideoxyU/ |
| | | | GG*/3deoxyU/ |
| GT-0U_F | Uni-GT_1 | 9 | GTTGGGTGGGTGGTGTGTGT |
| GT-1U_F | Uni-GT_1 | 52 | GTTGGGTGGGTGGTGTGTG*/3deoxyU/ |
| GT-2U_F | Uni-GT_1 | 53 | GTTGGGTGGG/ideoxyU/GGTGTGTG*/3deoxyU/ |
| GT-3U_F | Uni-GT_1 | 54 | GTTGGG/ideoxyU/GGGTGG/ideoxyU/GTGTG*/3deoxyU/ |
| GT-5U_F | Uni-GT_1 | 55 | GT/ideoxyU/GGG/ideoxyU/GGG/ideoxyU/GGTG/ideoxyU/ |
| | | | GTG*/3deoxyU/ |

Table 2. Universal primer sets for PCR amplification.

Target amplification reaction mixture components and reactions conditions are provided in Table 3 and Table 4, respectively. KAPA HiFi HotStart Uracil+ReadyMix (2×) comprising KAPA HiFi DNA polymerase, reaction buffer, dNTPs and MgCl₂ was obtained from Kapa Biosystems (Product #KK2802, Wilmington, Mass.).

TABLE 3

| Components | Volume (μL) | Final Concentration |
|---|---|---|
| H₂O | 45 | |
| KAPA Uracil+ 2x mix | 50 | 1x |
| UniX_F 20 uM | 1.5 | 0.3 uM |
| UniX_R 20 uM | 1.5 | 0.3 uM |
| DNA template (50 pg/μL) | 2 | |

Table 3. PCR amplification reaction mixture using modified primers.

TABLE 4

| Step | Temp (° C.) | Time |
|---|---|---|
| Initial Denaturation | 98 | 45 sec |
| 25 Cycles | 98 | 10 sec |
| | 57 | 15 sec |
| | (67 for GT primers) | |
| | 72 | 12 sec |
| Final Extension | 72 | 30 sec |

Table 4. Thermocycling conditions using modified primers.

The PCR reaction products comprised amplicons comprising a) a first amplicon nucleic acid strand comprising in 5′ to 3′ order: a forward primer sequence, a first target nucleic acid sequence, and a second adapter sequence and b) a second amplicon nucleic acid strand comprising in 5′ to 3′ order: a reverse primer sequence, a second target nucleic acid sequence, and a complementary first adapter sequence.

The PCR reaction amplicons comprising universal primers were optionally purified using, for example, a commercial purification kit such as QIAGEN PCR Purification Kit (e.g., QIAquick) or Promega PCR Clean-UP System (e.g., Wizard®). As a further option, the pH of the purified PCR reaction mixture was corrected by addition of 1/10 volume of 3 M sodium acetate, pH 5.

### Example 2: PCR Amplification of Target Sequences Using Universal Primers

A plurality of dsDNA molecules comprising two nucleic acid strands in a complementary base pair were amplified by PCR using pairs of primers comprising adapter sequences. The first nucleic acid strand in each dsDNA molecule comprised a first target sequence and the second nucleic acid in each dsDNA molecule comprised a second target sequence reverse complementary and base paired to the first target nucleic acid sequence. The first strand target nucleic acid sequences are shown in Table 5.

TABLE 5

| | SEQ ID | |
|---|---|---|
| Name | NO. | Target Sequence |
| gBlock | 56 | ATCTGAAGCGGAGTCCACACAACAAGATCGGGTTAATTGAATGCTCA |
| 340 | | GATGGTGCGCCGTCTGTCCTCTCTCTTGTCCTCTCCACTATCTCCACTA |
| | | CGCCTTAGGACCAGCGGTGAGCTATGTCTGAGGTCACCTACGGACCC |
| | | GGTTTATGTGAGCAACCAGAATTCGCCTTGACTCGTCAGCACGTTATT |
| | | AAAGCTATTGCTATGTTTGCGGCGGACACTTATCCTGGTGTCGTACTT |
| | | TCCTCGTGAAATCGCGTTCTGAGCTGGATCCCTCTGAGCCTTACGGAT |
| | | CAGTAGCCAGAGATTGTTGCTACTAGGCTCCGAGAAGAGACTCGAAG |
| | | CAGGAT |
| gBlock | 57 | ATCTGAAGCGGAGTCCACACAACAAGATCGGGTTAATTGAATGCTCA |
| 700 | | GATGGTGCGCCGTCTGTCCTCTCTCTTGTCCTCTCCACTATCTCCACTA |
| | | CGCCTTAGGACCAGCGGTGAGCTATGTCTGAGGTCACCTACGGACCC |
| | | GGTTTATGTGAGCAACCAGAATTCGCCTTGACTCGTCAGCACGTTATT |
| | | AAAGCTATTGCTATGTTTGCGGCGGACACTTATCCTGGTGTCGTACTT |
| | | TCCTCGTGAAATCGCGTTCTGCGTTGTCTTGGGTAGCTATATGGACTA |
| | | GTGTATACGATCCTTTGAGGAGCTAGAGGCGGTCACTGAACTCCTCTT |
| | | AATCCTGACACAATATGGAGGTGGCTGGCTCGAATCGACCAACCTGC |
| | | GCCGCCTGAGCCGACCTACGGACTACTCGTGCAGTGAGCTCTTTATTT |
| | | ATTAGTTCGCGCTACCTTTGATACAGGGCAAACATACTCAAGCTTTGT |
| | | ATTCACTCGCCGTCCCTTCAACGCCGCCTATGTTAACGAGATTAATGC |
| | | AACCTTAGGTCCGCAGCACACGCATAAGGGATTGGTGTATGTGGGCG |
| | | CCCAACCACAATGCGTGCGCAGTTAGAAAGCAATTAAGTGGGACTGG |
| | | AGCTGGATCCCTCTGAGCCTTACGGATCAGTAGCCAGAGATTGTTGCT |
| | | ACTAGGCTCCGAGAAGAGACTCGAAGCAGGAT |

Table 5. Target nucleic acid sequences.

The dsDNA molecules comprising a first target nucleic acid sequence from Table 5 and its reverse complement were amplified using a set of primers comprising adapter sequences to generate amplicons comprising said adapter sequences. Each forward primer comprised a first adapter sequence and a sequence from a first target sequence. Each reverse primer comprised a second adapter sequence and a sequence reverse complementary to the first target sequence. Table 6 provides sets of primers comprising adapter sequences used to amplify the target dsDNA molecules of Table 5. The adapter sequences are underlined.

TABLE 6

| | SEQ ID | |
|---|---|---|
| Name | NO. | Target Sequence |
| Uni5_PS2306 F | 58 | GCCGTACTCTCAACTCACATATCTGAAGCG |
| | | GAGTCCAC |
| Uni5_PS2307 R | 59 | GTTGTATCGTAGCCTGGTCTATCCTGCTTC |
| | | GAGTCTCTTCT |
| Uni6_PS2306 F | 60 | GGACTTGCTATGCTACGTGTATCTGAAGCG |
| | | GAGTCCAC |
| Uni6_PS2307 R | 61 | GAATCATTGCCCTACGGTCTATCCTGCTTC |
| | | GAGTCTCTTCT |

Table 6. Adaptor primer sequences.

The dsDNA target molecules were amplified by PCR using a set of primers from Table 6. Amplification reaction mixture components and reactions conditions used are provided in Table 7 and Table 8, respectively.

TABLE 7

| Components | Volume (μL) | Final Concentration |
|---|---|---|
| H2O | 11 | |
| Q5 polymerase | 25 | 1x |
| 2x Q5 Master Mix | 12.5 | 1x |
| F primer 20 μM (Uni5_PS2306 | 0.625 | 0.5 μM |
| or Uni6_PS2306) | | |
| R primer 20 μM | 0.625 | 0.5 μM |
| (Uni5_PS2307 or Uni6_PS2307) | | |
| DNA template (1 ng/μL) | 0.25 | 20 nM |

Table 7. PCR amplification reaction mixture using primers comprising adapter sequences.

TABLE 8

| Step | Temp (° C.) | Time |
|---|---|---|
| Initial Denaturation | 98 | 30 sec |
| 20 Cycles | 98 | 10 sec |
| | 62 | 15 sec |
| | 72 | 15 sec |
| Final Extension | 72 | 5 min |

Table 8. Thermocycling conditions used for appending adapter sequences to target nucleic acids.

The PCR reaction products comprised a) a first adapter amplicon nucleic acid strand comprising in 5′ to 3′ order: a forward adapter primer sequence, a first target nucleic acid sequence, a sequence reverse complementary to a reverse adapter primer sequence and b) a second adapter amplicon nucleic acid strand comprising in 5′ to 3′ order: a reverse adapter primer sequence, a second target nucleic acid sequence, and a sequence reverse complementary to a forward adapter primer sequence. The first adapter amplicon nucleic acid strand sequences are shown in Table 9. Adapter sequences and sequences reverse complementary to adapter sequences are underlined.

TABLE 9

| | SEQ ID | |
|---|---|---|
| Name | NO. | First adapter amplicon nucleic acid sequence |
| Uni5_PS | 62 | GCCGTACTCTCAACTCACATATCTGAAGCGGAGTCCACACAACA |
| 2306-340 | | AGATCGGGTTAATTGAATGCTCAGATGGTGCGCCGTCTGTCCTCT |
| first strand | | CTCTTGTCCTCTCCACTATCTCCACTACGCCTTAGGACCAGCGGT |
| | | GAGCTATGTCTGAGGTCACCTACGGACCCGGTTTATGTGAGCAAC |
| | | CAGAATTCGCCTTGACTCGTCAGCACGTTATTAAAGCTATTGCTA |
| | | TGTTTGCGGCGGACACTTATCCTGGTGTCGTACTTTCCTCGTGAA |
| | | ATCGCGTTCTGAGCTGGATCCCTCTGAGCCTTACGGATCAGTAGC |
| | | CAGAGATTGTTGCTACTAGGCTCCGAGAAGAGACTCGAAGCAGG |
| | | ATAGACCAGGCTACGATACAAC |
| Uni6_PS2 | 63 | GGACTTGCTATGCTACGTGTATCTGAAGCGGAGTCCACACAACA |
| 306-340 | | AGATCGGGTTAATTGAATGCTCAGATGGTGCGCCGTCTGTCCTCT |
| first strand | | CTCTTGTCCTCTCCACTATCTCCACTACGCCTTAGGACCAGCGGT |
| | | GAGCTATGTCTGAGGTCACCTACGGACCCGGTTTATGTGAGCAAC |
| | | CAGAATTCGCCTTGACTCGTCAGCACGTTATTAAAGCTATTGCTA |
| | | TGTTTGCGGCGGACACTTATCCTGGTGTCGTACTTTCCTCGTGAA |
| | | ATCGCGTTCTGAGCTGGATCCCTCTGAGCCTTACGGATCAGTAGC |
| | | CAGAGATTGTTGCTACTAGGCTCCGAGAAGAGACTCGAAGCAGG |
| | | ATAGACCGTAGGGCAATGATTC |
| Uni5_PS | 64 | GCCGTACTCTCAACTCACATATCTGAAGCGGAGTCCACACAACA |
| 2306-700 | | AGATCGGGTTAATTGAATGCTCAGATGGTGCGCCGTCTGTCCTCT |
| first strand | | CTCTTGTCCTCTCCACTATCTCCACTACGCCTTAGGACCAGCGGT |
| | | GAGCTATGTCTGAGGTCACCTACGGACCCGGTTTATGTGAGCAAC |
| | | CAGAATTCGCCTTGACTCGTCAGCACGTTATTAAAGCTATTGCTA |
| | | TGTTTGCGGCGGACACTTATCCTGGTGTCGTACTTTCCTCGTGAA |
| | | ATCGCGTTCTGCGTTGTCTTGGGTAGCTATATGGACTAGTGTATA |
| | | CGATCCTTTGAGGAGCTAGAGGCGGTCACTGAACTCCTCTTAATC |
| | | CTGACACAATATGGAGGTGGCTGGCTCGAATCGACCAACCTGCG |
| | | CCGCCTGAGCCGACCTACGGACTACTCGTGCAGTGAGCTCTTTAT |
| | | TTATTAGTTCGCGCTACCTTTGATACAGGGCAAACATACTCAAGC |
| | | TTTGTATTCACTCGCCGTCCCTTCAACGCCGCCTATGTTAACGAG |
| | | ATTAATGCAACCTTAGGTCCGCAGCACACGCATAAGGGATTGGT |
| | | GTATGTGGGCGCCCAACCACAATGCGTGCGCAGTTAGAAAGCAA |
| | | TTAAGTGGGACTGGAGCTGGATCCCTCTGAGCCTTACGGATCAGT |
| | | AGCCAGAGATTGTTGCTACTAGGCTCCGAGAAGAGACTCGAAGC |
| | | AGGATAGACCAGGCTACGATACAAC |
| Uni6_PS | 65 | GGACTTGCTATGCTACGTGTATCTGAAGCGGAGTCCACACAACA |
| 2306-700 | | AGATCGGGTTAATTGAATGCTCAGATGGTGCGCCGTCTGTCCTCT |
| first strand | | CTCTTGTCCTCTCCACTATCTCCACTACGCCTTAGGACCAGCGGT |
| | | GAGCTATGTCTGAGGTCACCTACGGACCCGGTTTATGTGAGCAAC |
| | | CAGAATTCGCCTTGACTCGTCAGCACGTTATTAAAGCTATTGCTA |
| | | TGTTTGCGGCGGACACTTATCCTGGTGTCGTACTTTCCTCGTGAA |
| | | ATCGCGTTCTGCGTTGTCTTGGGTAGCTATATGGACTAGTGTATA |
| | | CGATCCTTTGAGGAGCTAGAGGCGGTCACTGAACTCCTCTTAATC |
| | | CTGACACAATATGGAGGTGGCTGGCTCGAATCGACCAACCTGCG |
| | | CCGCCTGAGCCGACCTACGGACTACTCGTGCAGTGAGCTCTTTAT |
| | | TTATTAGTTCGCGCTACCTTTGATACAGGGCAAACATACTCAAGC |
| | | TTTGTATTCACTCGCCGTCCCTTCAACGCCGCCTATGTTAACGAG |
| | | ATTAATGCAACCTTAGGTCCGCAGCACACGCATAAGGGATTGGT |
| | | GTATGTGGGCGCCCAACCACAATGCGTGCGCAGTTAGAAAGCAA |
| | | TTAAGTGGGACTGGAGCTGGATCCCTCTGAGCCTTACGGATCAGT |
| | | AGCCAGAGATTGTTGCTACTAGGCTCCGAGAAGAGACTCGAAGC |
| | | AGGATAGACCGTAGGGCAATGATTC |

Table 9. PCR amplicons comprising adapter sequences.

The PCR amplicons comprising adapter sequences were PCR amplified using modified primers having sequences complementary to the adapter sequences, where the modified primers comprise one or more bases that have been replaced with uracils. Two sets of modified primers are shown in Table 10. An internal uracil is denoted by “ideoxyU”. A 3′ uracil is denoted by “3deoxyU”. The PCR amplicons having adapter sequences were PCR amplified using the primers of Table 10 to generate amplicons comprising a plurality of uracil bases.

TABLE 10

| | SEQ | |
|---|---|---|
| | ID | |
| Primer Name | NO. | Sequence (5′ to 3′) |
| Uni5_PS2306U F | 66 | GCCG/ideoxyU/ACTC/ideoxyU/CAAC/ |
| | | ideoxyU/CACA/3deoxyU/ |
| Uni5_PS2307U R | 67 | GTTG/ideoxyU/ATCG/ideoxyU/AGCC/ |
| | | ideoxyU/GGTC/3deoxyU/ |
| Uni6_PS2306U F | 68 | GGAC/ideoxyU/TGC/ideoxyU/ATGC/ |
| | | ideoxyU/ACG/ideoxyU/G/3deoxyU/ |
| Uni6_PS2307U R | 69 | GAA/ideoxyU/CAT/ideoxyU/GCCC/ |
| | | ideoxyU/ACGG/ideoxyU/C/3deoxyU/ |

Table 10. Universal primer sets comprising uracils.

The PCR amplicons comprising adapter sequences were PCR amplified using the primers of Table 10 and the mixture components and reactions conditions provided in Table 11 and Table 12, respectively.

TABLE 11

| Components | Volume (μL) | Final Concentration |
|---|---|---|
| H₂O | 35.5 | |
| 5x Phusion HF | 10 | 1x |
| Phusion U (2U/μL) | 0.5 | 1U/50 μL |
| 10 mM dNTP | 1 | 200 μM |
| UniX_340-F 20 μM | 1.25 | 0.5 μM |
| UniX_340-R 20 μM | 1.25 | 0.5 μM |
| DNA template (100x dilution | 0.5 | |
| from previous PCR reaction) | | |

Table 11. PCR amplification reaction mixture using universal primers.

TABLE 12

| Step | Temp (° C.) | Time |
|---|---|---|
| Initial Denaturation | 98 | 30 sec |
| 20 Cycles | 98 | 10 sec |
| | 60 | 15 sec |
| | 72 | 15 sec |
| Final Extension | 72 | 5 min |

Table 12. Thermocycling conditions used with universal primers.

The PCR reaction products amplified with uracil-containing primers comprised a) a first uracil amplicon nucleic acid strand comprising in 5′ to 3′ order: a forward universal primer sequence (e.g., Uni5_PS2306U), a first target nucleic acid sequence, and a sequence reverse complementary to a reverse adapter primer sequence and b) a second uracil amplicon nucleic acid strand comprising in 5′ to 3′ order: a reverse universal primer sequence (e.g., Uni5_PS2307U), a second target nucleic acid sequence, and a sequence reverse complementary to a forward adapter primer sequence. The first uracil amplicon nucleic acid strand sequences are shown in Table 13.

TABLE 13

| | SEQ ID | |
|---|---|---|
| Name | NO. | First uracil amplicon nucleic acid sequence |
| Uni5_PS2 | 70 | GCCG/ideoxyU/ACTC/ideoxyU/CAAC/ideoxyU/CACA/ideoxyU/ATCTG |
| 306U-340 | | AAGCGGAGTCCACACAACAAGATCGGGTTAATTGAATGCTCAGA |
| first strand | | TGGTGCGCCGTCTGTCCTCTCTCTTGTCCTCTCCACTATCTCCACT |
| | | ACGCCTTAGGACCAGCGGTGAGCTATGTCTGAGGTCACCTACGGA |
| | | CCCGGTTTATGTGAGCAACCAGAATTCGCCTTGACTCGTCAGCAC |
| | | GTTATTAAAGCTATTGCTATGTTTGCGGCGGACACTTATCCTGGT |
| | | GTCGTACTTTCCTCGTGAAATCGCGTTCTGAGCTGGATCCCTCTGA |
| | | GCCTTACGGATCAGTAGCCAGAGATTGTTGCTACTAGGCTCCGAG |
| | | AAGAGACTCGAAGCAGGATAGACCAGGCTACGATACAAC |
| Uni6_PS2 | 71 | GGAC/ideoxyU/TGC/ideoxyU/ATGC/ideoxyU/ACG/ideoxyU/G/ideoxyU/ |
| 306U-340 | | ATCTGAAGCGGAGTCCACACAACAAGATCGGGTTAATTGAATGC |
| first strand | | TCAGATGGTGCGCCGTCTGTCCTCTCTCTTGTCCTCTCCACTATCT |
| | | CCACTACGCCTTAGGACCAGCGGTGAGCTATGTCTGAGGTCACCT |
| | | ACGGACCCGGTTTATGTGAGCAACCAGAATTCGCCTTGACTCGTC |
| | | AGCACGTTATTAAAGCTATTGCTATGTTTGCGGCGGACACTTATC |
| | | CTGGTGTCGTACTTTCCTCGTGAAATCGCGTTCTGAGCTGGATCCC |
| | | TCTGAGCCTTACGGATCAGTAGCCAGAGATTGTTGCTACTAGGCT |
| | | CCGAGAAGAGACTCGAAGCAGGATAGACCGTAGGGCAATGATTC |
| Uni5_PS2 | 72 | GCCG/ideoxyU/ACTC/ideoxyU/CAAC/ideoxyU/CACA/ideoxyU/ATCTG |
| 306U-700 | | AAGCGGAGTCCACACAACAAGATCGGGTTAATTGAATGCTCAGA |
| first strand | | TGGTGCGCCGTCTGTCCTCTCTCTTGTCCTCTCCACTATCTCCACT |
| | | ACGCCTTAGGACCAGCGGTGAGCTATGTCTGAGGTCACCTACGGA |
| | | CCCGGTTTATGTGAGCAACCAGAATTCGCCTTGACTCGTCAGCAC |
| | | GTTATTAAAGCTATTGCTATGTTTGCGGCGGACACTTATCCTGGT |
| | | GTCGTACTTTCCTCGTGAAATCGCGTTCTGCGTTGTCTTGGGTAGC |
| | | TATATGGACTAGTGTATACGATCCTTTGAGGAGCTAGAGGCGGTC |
| | | ACTGAACTCCTCTTAATCCTGACACAATATGGAGGTGGCTGGCTC |
| | | GAATCGACCAACCTGCGCCGCCTGAGCCGACCTACGGACTACTCG |
| | | TGCAGTGAGCTCTTTATTTATTAGTTCGCGCTACCTTTGATACAGG |
| | | GCAAACATACTCAAGCTTTGTATTCACTCGCCGTCCCTTCAACGC |
| | | CGCCTATGTTAACGAGATTAATGCAACCTTAGGTCCGCAGCACAC |
| | | GCATAAGGGATTGGTGTATGTGGGCGCCCAACCACAATGCGTGC |
| | | GCAGTTAGAAAGCAATTAAGTGGGACTGGAGCTGGATCCCTCTG |
| | | AGCCTTACGGATCAGTAGCCAGAGATTGTTGCTACTAGGCTCCGA |
| | | GAAGAGACTCGAAGCAGGATAGACCAGGCTACGATACAAC |
| Uni6_PS2 | 73 | GGAC/ideoxyU/TGC/ideoxyU/ATGC/ideoxyU/ACG/ideoxyU/G/ideoxyU/ |
| 306U-700 | | ATCTGAAGCGGAGTCCACACAACAAGATCGGGTTAATTGAATGC |
| first strand | | TCAGATGGTGCGCCGTCTGTCCTCTCTCTTGTCCTCTCCACTATCT |
| | | CCACTACGCCTTAGGACCAGCGGTGAGCTATGTCTGAGGTCACCT |
| | | ACGGACCCGGTTTATGTGAGCAACCAGAATTCGCCTTGACTCGTC |
| | | AGCACGTTATTAAAGCTATTGCTATGTTTGCGGCGGACACTTATC |
| | | CTGGTGTCGTACTTTCCTCGTGAAATCGCGTTCTGCGTTGTCTTGG |
| | | GTAGCTATATGGACTAGTGTATACGATCCTTTGAGGAGCTAGAGG |
| | | CGGTCACTGAACTCCTCTTAATCCTGACACAATATGGAGGTGGCT |
| | | GGCTCGAATCGACCAACCTGCGCCGCCTGAGCCGACCTACGGACT |
| | | ACTCGTGCAGTGAGCTCTTTATTTATTAGTTCGCGCTACCTTTGAT |
| | | ACAGGGCAAACATACTCAAGCTTTGTATTCACTCGCCGTCCCTTC |
| | | AACGCCGCCTATGTTAACGAGATTAATGCAACCTTAGGTCCGCAG |
| | | CACACGCATAAGGGATTGGTGTATGTGGGCGCCCAACCACAATG |
| | | CGTGCGCAGTTAGAAAGCAATTAAGTGGGACTGGAGCTGGATCC |
| | | CTCTGAGCCTTACGGATCAGTAGCCAGAGATTGTTGCTACTAGGC |
| | | TCCGAGAAGAGACTCGAAGCAGGATAGACCGTAGGGCAATGATTC |

Table 13. PCR amplicon sequences comprising uracils.

The PCR reaction amplicons comprising universal primers were optionally purified using Promega PCR Clean-UP System (e.g., Wizard®).

### Example 3: Primer Removal from PCR Amplicons Comprising Universal Primers

PCR amplicon products comprising uracils from Examples 1 and 2 were purified and then enzymatically treated with DNA repair enzymes to initiate the removal of primer and adapter sequences by excision of the modified uracil bases. The amplicon products were combined in a cleavage reaction comprising endonuclease VIII (Endo VIII) and uracil DNA glycosylase (UDG). The cleavage reaction components are shown in Table 14. 10× CutSmart® was obtained from New England BioLabs (Product # B7204S). UDG and Endo VIII were obtained from Enzymatics (Product # Y9180L). The cleavage reactions occurred at 37° C. for 1 hour (Example 1 amplicons) or 1.5 hours (Example 2 amplicons).

TABLE 14

| | | Example 2 | Example 2 |
|---|---|---|---|
| | Example 1 | amplicons having | amplicons having |
| | amplicons | Uni5_PS2306U | Uni6_PS2306U |
| | (volume | primers | primers |
| Components | in μL) | (volume in μL) | (volume in μL) |
| H₂O | 6 | 295.64 | 306.28 |
| 10x Buffer | 2 | 48 | 48 |
| (CutSmart ® | | | |
| or ThermoPol) | | | |
| PCR amplicon | 10 | 88.36 (12 μg) | 77.72 (12 μg) |
| | (100 ng/μL) | | |
| Endo VIII | 1 | 24 | 24 |
| (Enzymatics) | | | |
| UDG (Enzymatics) | 1 | 24 | 24 |
| (Final Volume) | (20) | (480) | (480) |

Table 14. Primer cleavage reaction mixture.

Following the excision of uracils from the PCR amplicons, the cleaved amplicons from Example 1 were combined with an equal volume (20 μL) of PCR mixture A (8 μL 5× Kapa buffer, 0.8 μL 10 mM dTNPs, 0.8 μL Kapa HiFi at 1 U/μL, 10.4 μL water) and incubated at 70° C. for 1 hour. The exonuclease activity of the Kapa HiFi DNA polymerase removed the primer and adapter bases downstream from the excision sites. The reactions were optionally purified using a commercial PCR clean up kit.

Following excision of uracils from the PCR amplicons, aliquots of the cleaved amplicons from Example 2 were combined with Deep Vent DNA Polymerase from NEB (final concentration 2 U/50 μL) and various amounts of dNTPs (final concentrations between 100 μM and 200 μM). The aliquots were incubated at 75° C. for 60 min, 90 min, or 120 min to melt off the primers and adapter sequences, followed by a final extension at 68° C. for 15 min.

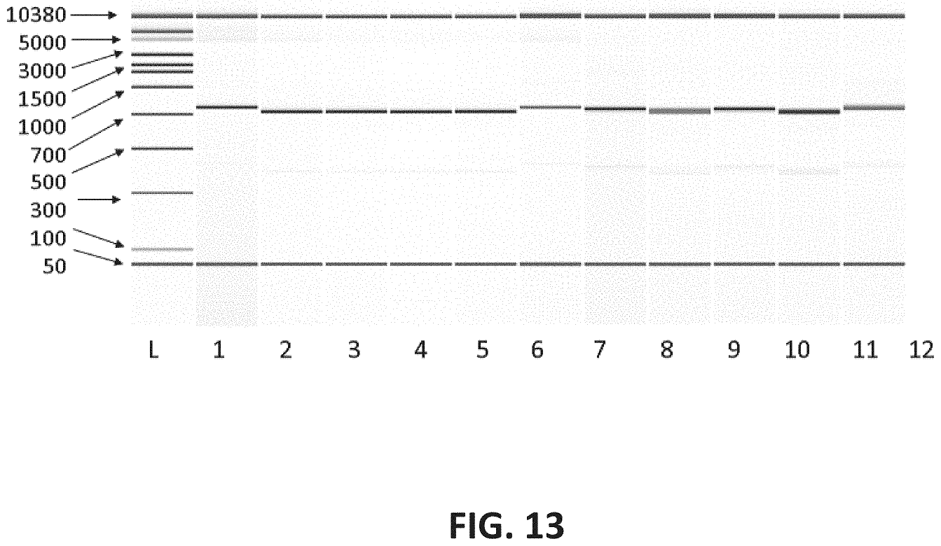

Particular PCR amplicon products comprising Uni6_PS2306-700 (first strand sequence shown in Table 9) were prepared by PCR using the conditions described in Tables 7 and 8, where the extension time during the 20 cycles was increased from 15 seconds to 30 seconds. These amplicon products were further amplified using the Uni6_PS2306U F and Uni6_PS2307U R primers from Table 10 with slight modifications to the amplification reaction conditions of Tables 11 and 12 (e.g., primer concentration of 0.2 μM and cycle extension time was increased to 30 seconds). Either Phusion or Kapa enzymes were used for amplification with the universal primers. The amplicons comprising uracils (100 μL crude product) were then treated with EndoVIII (5 μL) and UDG (5 μL) for 90 min at 37° C. without amplicon purification. Following digestion, some digested samples were supplemented with Phusion DNA polymerase and incubated at 75° C. for 90 min or 120 min, followed by a final incubation at 72° C. for 15 min. The digested amplicons were visualized using gel electrophoresis and are shown in FIG. 13, with lane descriptions provided in Table 15.

TABLE 15

| Lane | 1 | 2 | 3 | 4 | 5 | 6 | 7 | 8 | 9 | 10 | 11 |
|---|---|---|---|---|---|---|---|---|---|---|---|
| DNA | K | K | K | K | K | P | P | P | P | P | P |
| Polymerase | | | | | | | | | | | |
| USER | — | + | + | + | + | + | + | + | + | + | + |
| Supplement | N/A | — | + | — | + | N/A | — | + | — | + | N/A |
| with DNA | | | | | | | | | | | |
| Polymerase | | | | | | | | | | | |
| Reaction time | N/A | 90 | 90 | 120 | 120 | N/A | 90 | 90 | 120 | 120 | N/A |
| (min) | | | | | | | | | | | |

Table 15. Primer removal from crude PCR products. Abbreviations: P=Phusion, K=Kappa

### Example 4: Visualization of Universal Primer Removal

The efficiency of universal primer removal was tested by first performing modified methods of Examples 1 and 3, followed by visualization of reaction products by gel electrophoresis.

DNA duplexes comprising a first target sequence in Table 1 of Example 1 and a second target sequence reverse complementary to the first target sequence, were amplified using a set of primers selected from Table 2 of Example 1. The set of primers were selected so that only one of the primers in the primer pair comprised a uracil base. For example, primer GT-4U_R was paired with GT-0U_F. The DNA duplexes were amplified using the reaction conditions described in Tables 3 and 4 of Example 1. A list of primer pairs used to amplify the DNA duplexes from is shown in Table 16.

TABLE 16

| Primer Set | | |
|---|---|---|
| Number | Primer 1 | Primer 2 |
| 1 | Uni5-1U_F | Uni5-0U_R |
| 2 | Uni5-2U_F | Uni5-0U_R |
| 3 | Uni5-4U_F | Uni5-0U_R |
| 4 | UNI5-1U_R | Uni5-0U_F |
| 5 | UNI5-2U_R | Uni5-0U_F |
| 6 | UNI5-4U_R | Uni5-0U_F |
| 7 | Uni6v2-1U_F | Uni6v2-0U_R |
| 8 | Uni6v2-2U_F | Uni6v2-0U_R |
| 9 | Uni6v2-3U_F | Uni6v2-0U_R |
| 10 | Uni6v2-5U_F | Uni6v2-0U_R |
| 11 | Uni6v1-1U_R | Uni6v2-0U_F |
| 12 | Uni6v1-3U_R | Uni6v2-0U_F |
| 13 | Uni6v1-5U_R | Uni6v2-0U_F |
| 14 | Uni7-1U_F | Uni7-0U_R |
| 15 | Uni7-3U_F | Uni7-0U_R |
| 16 | Uni7-1U_R | Uni7-0U_F |
| 17 | Uni7-3U_R | Uni7-0U_F |
| 18 | Uni8-1U_F | Uni8-0U_R |
| 19 | Uni8-3U_F | Uni8-0U_R |
| 20 | Uni8-1U_R | Uni8-0U_F |
| 21 | Uni8-3U_R | Uni8-0U_F |
| 22 | GT-1U_F | GT-0U_R |
| 23 | GT-2U_F | GT-0U_R |
| 24 | GT-3U_F | GT-0U_R |
| 25 | GT-5U_F | GT-0U_R |
| 26 | GT-1U_R | GT-0U_F |
| 27 | GT-2U_R | GT-0U_F |
| 28 | GT-3U_R | GT-0U_F |
| 29 | GT-4U_R | GT-0U_F |
| 30 | GT-6U_R | GT-0U_F |

Table 16. Universal primer sets comprising a first primer having a uracil base and a second primer not having a uracil base.

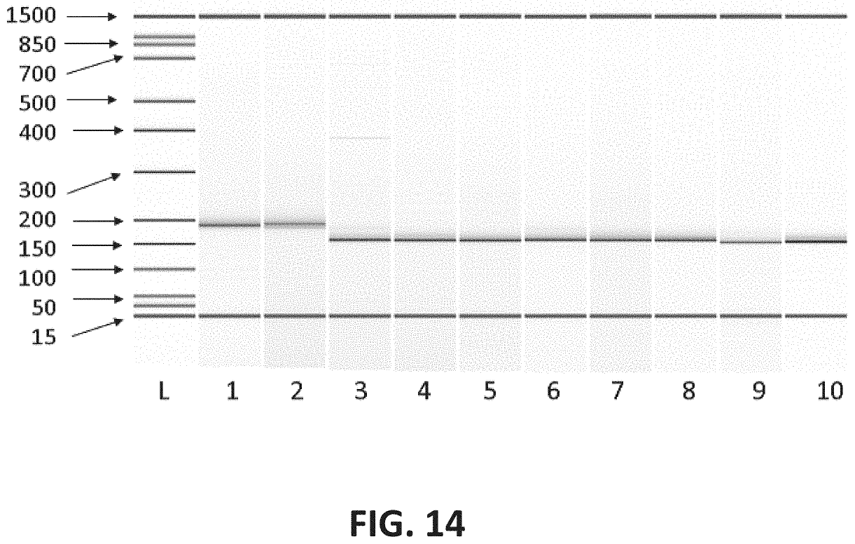

The PCR reaction amplicons comprised either a) a first strand comprising a uracil base and a second strand not comprising a uracil base or b) a first strand not comprising a uracil base and a second strand comprising a uracil base. The PCR reaction amplicons were treated with DNA repair enzymes as described in Example 3. Briefly, a sample of each PCR reaction product was combined with 10× buffer, EndoIII, UDG and water, and incubated at 37° C. for 1 hour. Following treatment with the DNA repair enzymes, the samples were supplemented with dNTPs, Kapa HiFi DNA polymerase and Kapa HiFi buffer, as described in Example 3. The digested amplicons were visualized by gel electrophoresis for a decrease in product size. FIG. 14 is an image capture of gel showing a decrease in product size after universal primer removal. Table 17 provides a legend for each lane of FIG. 14.

TABLE 17

| Lane | Primer Set | |
|---|---|---|
| Number | Number | Remarks |
| L | — | Marker |
| 1 | 5 (not treated) | Control product, not treated with DNA |
| | | repair enzymes |
| 2 | 6 (not treated) | Control product, not treated with DNA |
| | | repair enzymes |
| 3 | 1 | Decrease in product size after enzymatic |
| | | treatment indicating primer removal |
| 4 | 2 | Decrease in product size |
| 5 | 3 | Decrease in product size |
| 6 | 4 | Decrease in product size |
| 7 | 5 | Decrease in product size |
| 8 | 6 | Decrease in product size |
| 9 | 7 | Decrease in product size |
| 10 | 8 | Decrease in product size |

### Table 17. Legend for FIG. 14.

### Example 5: Measurement of Universal Primer Removal Efficiency

The efficiency of universal primer removal tested in Example 4 was further analyzed by next generation sequencing. The digested amplicons of Example 4 were each cloned into a TOPO per4 blunt vector, amplified by PCR using primers having barcodes for sequencing, and then sequenced using MiSeq Gene & Small Genome Sequencer (Illumina). Amplicons that had greater than 90% primer sequence removal during a sequencing read are shown in Table 18.

TABLE 18

| Amplicon | |
|---|---|
| primer set | Uracil primer |
| 24 | GT-3U_F |
| 27 | GT-2U_R |
| 1 | Uni5-1U_F |
| 4 | UNI5-1U_R |
| 5 | UNI5-2U_R |
| 9 | Uni6v2-3U_F |
| 14 | Uni7-1U_F |
| 15 | Uni7-3U_F |
| 16 | Uni7-1U_R |
| 17 | Uni7-3U_R |
| 19 | Uni8-3U_F |
| 21 | Uni8-3U_R |

Table 18. DNA amplicons having at least 90% primer sequence removal.

### Example 6: Measurement of Universal Primer Removal Efficiency

The efficiency of universal primer removal was tested by first performing amplification and digestion methods described in Examples 1-3, followed by analyzing digested amplicons for primer removal using next generation sequencing. DNA duplexes comprising the target sequence gBlock 340 (Table 5, Example 2) and its reverse complement were amplified using pairs of primers listed in Table 19. The DNA duplexes were amplified using modifications to the reaction conditions described in Tables 3 and 4 of Example 1, which are briefly referenced in Table 19. The PCR reaction amplicons were treated with DNA repair enzymes to remove universal primers by performing the cleavage reactions described in Example 3, with various modifications referenced in Table 19.

TABLE 19

| Sample | | Primer Sequences | |
|---|---|---|---|
| Number | Sample Name | (Forward and Reverse) | Reaction Conditions |
| Smp01 | Uni6-2U_Taq_EM- | Uni6-2U-F: | Target nucleic acid sequence |
| | USER_75C-60m- | GGA CTT GCT A/ideoxyU/G | amplified using Taq polymerase. |
| | cyc | CTA CGT G/3deoxyU/ (SEQ | USER enzyme from NEB was used |
| | | ID NO: 74) | to digest the amplicons. |
| | | Uni6-2U-R: | Thermocycling was performed |
| | | GAA TCA T/ideoxyU/G CCC | after PCR and enzyme digestion. |
| | | TAC GGT C/3deoxyU/ (SEQ | The thermocycling extension time |
| | | ID NO: 75) | was 60 min at 75° C. |
| Smp02 | Uni6*U_Kapa_EM- | Uni6-2*U_F: | Target nucleic acid sequence |
| | USER_75C-60m- | GGA CTT GCT A/ideoxyU/G | amplified using Kapa Uracil |
| | cyc | CTA CGT G*/3deoxyU/ (SEQ | polymerase. USER enzyme from |
| | | ID NO: 74) | NEB was used to digest the |
| | | Uni603*U_R: | amplicons. Thermocycling was |
| | | GAA TCA /ideoxyU/TG CCC/ | performed after PCR and enzyme |
| | | ideoxyU/AC GGT | digestion. The thermocycling |
| | | C*/3deoxyU/ (SEQ ID NO: | extension time was 60 min at |
| | | 34) | 75∞ C. |
| Smp03 | Uni6_UA*T*C_Kapa_EM- | Uni-2U_F_A*T*C: | Target nucleic acid sequence |
| | USER_75C- | GGA CTT GCT A/ideoxyU/G | amplified using Kapa Uracil |
| | 60m-cyc | CTA CGT G/ideoxyU/A*T*C | polymerase. USER enzyme from |
| | | (SEQ ID NO: 77) | NEB was used to digest the |
| | | Uni6-3U_R_A*T*C: | amplicons. Thermocycling was |
| | | GAA TCA /ideoxyU/TG CCC/ | performed after PCR and enzyme |
| | | ideoxyU/AC GGT | digestion. The thermocycling |
| | | C/ideoxyU/A*T*C (SEQ ID | extension time was 60 min at |
| | | NO: 78) | 75° C. |
| Smp04 | Uni6-2U_Taq_EM- | Uni6-2U-F: | Target nucleic acid sequence |
| | USER_75C-60m | GGA CTT GCT A/ideoxyU/G | amplified using Taq polymerase. |
| | | CTA CGT G/3deoxyU/ (SEQ | USER enzyme from NEB was used |
| | | ID NO: 74) | to digest the amplicons. Digested |
| | | Uni6-2U-R: | amplicons were incubated for |
| | | GAA TCA T/ideoxyU/G CCC | 60 min at 75° C. |
| | | TAC GGT C/3deoxyU/ (SEQ | |
| | | ID NO: 75) | |
| Smp05 | Uni6*U_Kapa_EM- | Uni6-2*U_F: | Target nucleic acid sequence |
| | USER_75C-60m | GGA CTT GCT A/ideoxyU/G | amplified using Kapa polymerase. |
| | | CTA CGT G*/3deoxyU/ (SEQ | USER enzyme from NEB was used |
| | | ID NO: 74) | to digest the amplicons. Digested |
| | | Uni603*U_R: | amplicons were incubated for |
| | | GAA TCA /ideoxyU/TG CCC/ | 60 min at 75° C. |
| | | ideoxyU/AC GGT | |
| | | C*/3deoxyU/ (SEQ ID NO: | |
| | | 34) | |
| Smp06 | Uni6_UA*A*T_Kapa_EM- | Uni-2U_F_A*T*C: | Target nucleic acid sequence |
| | USER_75C- | GGA CTT GCT A/ideoxyU/G | amplified using Kapa polymerase. |
| | 60m | CTA CGT G/ideoxyU/A*T*C | USER enzyme from NEB was used |
| | | (SEQ ID NO: 77) | to digest the amplicons. Digested |
| | | Uni6-3U_R_A*T*C: | amplicons were incubated for |
| | | GAA TCA /ideoxyU/TG CCC/ | 60 min at 75° C. |
| | | ideoxyU/AC GGT | |
| | | C/ideoxyU/A*T*C (SEQ ID | |
| | | NO: 78) | |
| Smp07 | Uni6*U_75C-90m | Uni6-2*U_F: | Enzymatics EndoVIII and UDG |
| | | GGA CTT GCT A/ideoxyU/G | enzymes were used to digest the |
| | | CTA CGT G*/3deoxyU/ (SEQ | amplicons. Digested amplicons |
| | | ID NO: 74) | were incubated for 90 min at 75° C. |
| | | Uni603*U_R: | |
| | | GAA TCA /ideoxyU/TG CCC/ | |
| | | ideoxyU/AC GGT | |
| | | C*/3deoxyU/ (SEQ ID NO: | |
| | | 34) | |
| Smp08 | Uni6U- | Uni-2U_F_A*T*C: | Enzymatics EndoVIII and UDG |
| | A*T*C_75C-90m | GGA CTT GCT A/ideoxyU/G | enzymes were used to digest the |
| | | CTA CGT G/ideoxyU/A*T*C | amplicons. Digested amplicons |
| | | (SEQ ID NO: 77) | were incubated for 90 min at 75° C. |
| | | Uni6-3U_R_A*T*C: | |
| | | GAA TCA /ideoxyU/TG CCC/ | |
| | | ideoxyU/AC GGT | |
| | | C/ideoxyU/A*T*C (SEQ ID | |
| | | NO: 78) | |
| Smp09 | Uni6*U_72C-cyc- | Uni6-2*U_F: | Enzymatics EndoVIII and UDG |
| | 90m | GGA CTT GCT A/ideoxyU/G | enzymes were used to digest the |
| | | CTA CGT G*/3deoxyU/ (SEQ | amplicons. Thermocycling was |
| | | ID NO: 74) | performed after PCR and enzyme |
| | | Uni603*U_R: | digestion. The thermocycling |
| | | GAA TCA /ideoxyU/TG CCC/ | extension time was 90 min at |
| | | ideoxyU/AC GGT | 72° C. |
| | | C*/3deoxyU/ (SEQ ID NO: | |
| | | 34) | |
| Smp10 | Uni6U- | Uni-2U_F_A*T*C: | Enzymatics EndoVIII and UDG |
| | A*T*C_72C-cyc- | GGA CTT GCT A/ideoxyU/G | enzymes were used to digest the |
| | 90m | CTA CGT G/ideoxyU/A*T*C | amplicons. Digested amplicons |
| | | (SEQ ID NO: 77) | were incubated for 90 min at 72° C. |
| | | Uni6-3U_R_A*T*C: | |
| | | GAA TCA /ideoxyU/TG CCC/ | |
| | | ideoxyU/AC GGT | |
| | | C/ideoxyU/A*T*C (SEQ ID | |
| | | NO: 78) | |
| Smp11 | Uni6*U_Kapa_2x- | Uni6-2*U_F: | Target nucleic acid sequence |
| | EM-USER_75C- | GGA CTT GCT A/ideoxyU/G | amplified using Kapa Uracil |
| | 90m | CTA CGT G*/3deoxyU/ (SEQ | polymerase. USER enzyme from |
| | | ID NO: 74) | NEB was used to digest the |
| | | Uni603*U_R: | amplicons. Digested amplicons |
| | | GAA TCA /ideoxyU/TG CCC/ | were incubated for 90 min at 75° C. |
| | | ideoxyU/AC GGT | |
| | | C*/3deoxyU/ (SEQ ID NO: | |
| | | 34) | |
| Smp12 | Uni6*U_Kapa_2x- | Uni6-2*U_F: | Target nucleic acid sequence |
| | EM-USER_75C- | GGA CTT GCT A/ideoxyU/G | amplified using Kapa Uracil |
| | 90m-cyc | CTA CGT G*/3deoxyU/ (SEQ | polymerase. USER enzyme from |
| | | ID NO: 74) | NEB was used to digest the |
| | | Uni603*U_R: | amplicons. Thermocycling was |
| | | GAA TCA /ideoxyU/TG CCC/ | performed after PCR and enzyme |
| | | ideoxyU/AC GGT | digestion. The thermocycling |
| | | C*/3deoxyU/ (SEQ ID NO: | extension time was 90 min at |
| | | 34) | 75° C. |
| Smp13 | Pre-act-Kapa_72C- | Uni6-2*U_F: | Target nucleic acid sequence |
| | 60m | GGA CTT GCT A/ideoxyU/G | amplification using pre-activated |
| | | CTA CGT G*/3deoxyU/ (SEQ | Kapa Uracil+. Digested amplicons |
| | | ID NO: 74) | were incubated for 60 min at 72° C. |
| | | Uni603*U_R: | |
| | | GAA TCA /ideoxyU/TG CCC/ | |
| | | ideoxyU/AC GGT | |
| | | C*/3deoxyU/ (SEQ ID NO: | |
| | | 34) | |
| Smp14 | Pre-act- | Uni6-2*U_F: | Target nucleic acid sequence |
| | Kapa + Q5_72C-60m | GGA CTT GCT A/ideoxyU/G | amplification using pre-activated |
| | | CTA CGT G*/3deoxyU/ (SEQ | Kapa Uracil+. Digested amplicons |
| | | ID NO: 74) | were incubated for 60 min at 72° C. |
| | | Uni603*U_R: | with Q5. |
| | | GAA TCA /ideoxyU/TG CCC/ | |
| | | ideoxyU/AC GGT | |
| | | C*/3deoxyU/ (SEQ ID NO: | |
| | | 34) | |
| Smp15 | Pre-act- | Uni6-2*U_F: | Target nucleic acid sequence |
| | Phusion + Q5_72C- | GGA CTT GCT A/ideoxyU/G | amplification using pre-activated |
| | 60m | CTA CGT G*/3deoxyU/ (SEQ | Phusion. Digested amplicons were |
| | | ID NO: 74) | incubated for 60 min at 72° C. with |
| | | Uni603*U_R: | Q5. |
| | | GAA TCA /ideoxyU/TG CCC/ | |
| | | ideoxyU/AC GGT | |
| | | C*/3deoxyU/ (SEQ ID NO: | |
| | | 34) | |

Table 19. Universal primer sequences used in primer removal efficiency studies.

The digested amplicons were each cloned into a TOPO per4 blunt vector and then sequenced using MiSeq Gene & Small Genome Sequencer (Illumina). A summary of the primer removal efficiencies as well as target sequence truncations are shown in Table 20. A sample of the amplicons had 100% primer removal using the methods provided in the examples. Colony number legend: H>200; M>100 and <200; LM>50 and <100; L<50 colonies per plate.

TABLE 20

| | | Total | | | | | Total | | | | |
|---|---|---|---|---|---|---|---|---|---|---|---|
| | | valid | Perfect | | | | valid | Perfect | | | |
| Sample | Colony | Fwd | Fwd | Efficiency | Truncation | | Rev | Rev | Efficiency | Truncation | |
| number | # | seqs | End | (%) | 5′ end | Residue | seqs | End | (%) | 5′ end | Residue |
| Smp01 | H | 30 | 30 | 100 | 0 | 0 | 29 | 28 | 97 | 1 | 0 |
| Smp02 | H | 30 | 30 | 100 | 0 | 0 | 30 | 30 | 100 | 0 | 0 |
| Smp03 | L | 4 | 2 | 50 | 1 | 1 | 4 | 2 | 50 | 2 | 0 |
| Smp04 | H | 29 | 26 | 90 | 3 | 0 | 28 | 28 | 100 | 0 | 0 |
| Smp05 | H | 25 | 22 | 88 | 1 | 2 | 25 | 24 | 96 | 1 | 0 |
| Smp06 | L | 9 | 4 | 44 | 4 | 1 | 9 | 8 | 89 | 1 | 0 |
| Smp07 | LM | 31 | 29 | 94 | 0 | 2 | 30 | 29 | 97 | 0 | 1 |
| Smp08 | L | 0 | 0 | N/A | 0 | 0 | 0 | 0 | N/A | 0 | 0 |
| Smp09 | L | 28 | 19 | 68 | 2 | 7 | 29 | 29 | 100 | 0 | 0 |
| Smp10 | L | 7 | 6 | 86 | 1 | 0 | 7 | 4 | 57 | 2 | 1 |
| Smp11 | H | 30 | 20 | 67 | 2 | 8 | 30 | 30 | 100 | 0 | 0 |
| Smp12 | M | 5 | 2 | 40 | 1 | 2 | 5 | 3 | 60 | 2 | 0 |
| Smp13 | LM | 26 | 13 | 50 | 1 | 12 | 26 | 26 | 100 | 0 | 0 |
| Smp14 | L | 30 | 13 | 43 | 4 | 13 | 32 | 31 | 97 | 1 | 0 |
| Smp15 | L | 21 | 12 | 57 | 6 | 3 | 21 | 19 | 90 | 2 | 0 |

Table 20. Primer removal efficiency.

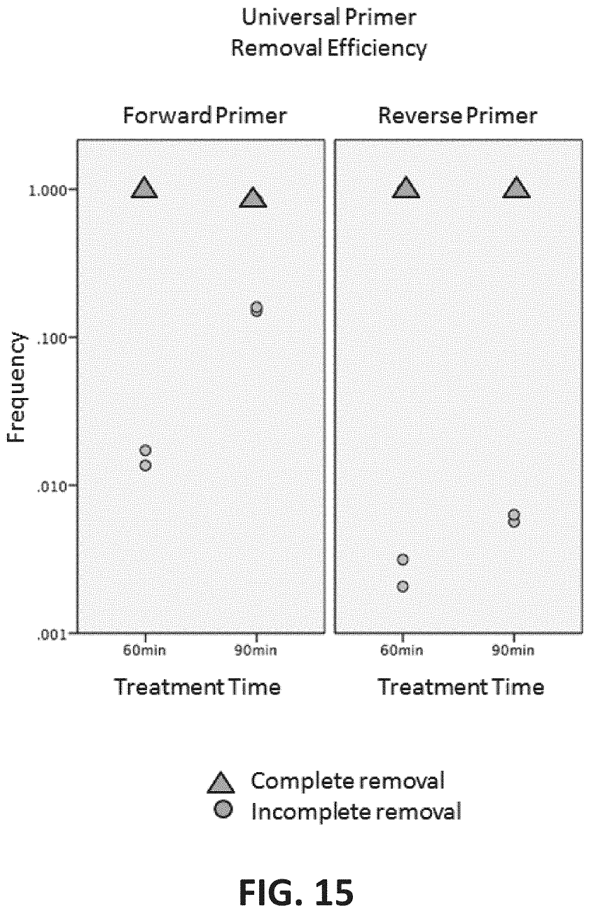

Primers from sample 02 in Table 19 were used to amplify target sequence gBlock 340. The primers were removed for either 60 min or 90 min using DNA repair enzymes. The digested amplicons were cloned into TOPO PCR4 blunt and ran on the MisSeq. The removal efficiency is shown in FIG. 15 and Table 21. Primer removal efficiency with 60 min digestion was 98%.

TABLE 21

| | | Primer | | | Total | |
|---|---|---|---|---|---|---|
| Treatment | Primer | removal | Search Pattern | Count | count | Frequency |
| 60 min | F | Complete | TTATCTGAAGCG | 3759 | 3826 | 0.982 |
| | | | (SEQ ID NO: 79) | | | |
| 60 min | F | Incomplete | GTATCTGAAGCG | 59 | 3826 | 0.015 |
| | | | (SEQ ID NO: 80) | | | |
| 60 min | R | Complete | TTATCCTGCTTC | 4138 | 4158 | 0.995 |
| | | | (SEQ ID NO: 81) | | | |
| 60 min | R | Incomplete | CTATCCTGCTTC | 11 | 4158 | 0.003 |
| | | | (SEQ ID NO: 82) | | | |
| 90 min | F | Complete | TTATCTGAAGCG | 1050 | 1245 | 0.843 |
| | | | (SEQ ID NO: 79) | | | |
| 90 min | F | Incomplete | GTATCTGAAGCG | 193 | 1245 | 0.155 |
| | | | (SEQ ID NO: 80) | | | |
| 90 min | R | Complete | TTATCCTGCTTC | 1330 | 1340 | 0.993 |
| | | | (SEQ ID NO: 81) | | | |
| 90 min | R | Incomplete | CTATCCTGCTTC | 8 | 1340 | 0.006 |
| | | | (SEQ ID NO: 82) | | | |

Table 21. Primer removal efficiency.

### Example 7: Universal Primer Selection for Nucleic Acid Amplification Methods

A plurality of universal primer pairs were generated having different numbers and configurations of uracil nucleobases. Each uracil nucleobase was separated by a subsequent or preceding nucleobase within a primer by four to ten non-uracil nucleobases. Each of the plurality of universal primers was used to amplify a target nucleic acid using PCR to extend each of the plurality of primers into amplicons of the target nucleic acid. Primer sequence was removed from the amplicons, and the efficiency of primer sequence removal for each of the plurality of universal primers was evaluated. A correlation between universal primer sequence removal efficiency and number and position of uracil nucleobases within the universal primer sequence was identified.

Universal Primers

Universal primers designed for target nucleic acid amplification are listed in Table 22, where primers having the same number are part of a single primer pair, and F indicates a forward primer and R indicates a reverse primer in the primer pair. Each universal primer comprises a 3′ terminal uracil nucleobase bound to the preceding nucleobase base a nuclease-resistant phosphorothioate bond.

TABLE 22

| Primer Name | SEQ ID NO. | Sequence |
|---|---|---|
| F03 with 1 U | 83 | TGTTCAGCGATCCGTCCACU |
| R03 with 1 U | 84 | TGCCACAGAGTTCCACCAGU |
| F04 with 1 U | 85 | AAGCCAGACCGTTCCACAAU |
| R04 with 1 U | 86 | GCCGTTACAGCCTAATCCGU |
| F06 with 1 U | 87 | GTGCCATTCCAACTCTCCGU |
| R06 with 1 U | 88 | GAAGCAAGCCTGTCTGCCTU |
| F07 with 1 U | 89 | GCTCTGTGGACGAAGGATGU |
| R07 with 1 U | 90 | TGCAAGCGTGTATGATGGCU |
| F10 with 2 U | 91 | AAGCTCAGCCUCCAGTTGCTCU |
| R10 with 2 U | 92 | ACACTCAGCGUTCCGTTACCGU |
| F13 with 2 U | 93 | CCGCACACCTUAGTTCGACGAU |
| R13 with 2 U | 94 | CGATGCCAAGUTCACCACCGTU |
| F16 with 2 U | 95 | CCAGCCAGTCUCCGTCAGTCAU |
| R16 with 2 U | 96 | GCCGCGTTATUCATACAGCCGU |
| F24 with 2 U | 97 | ACCAGTTCCGUTCCACCTGAGU |
| R24 with 2 U | 98 | CCTTCATCGTUGCCACCGAGAU |
| F28 with 3 U | 99 | GCCACATCUCTCAATCUGCCGTGU |
| R28 with 3 U | 100 | GGTCAATCUGCCGCAAUCCAGTCU |
| F32 with 3 U | 101 | CCACCAGTUGTCAGAGUTGTGCCU |
| R32 with 3 U | 102 | GTGAACGAUGCCAGAGUTGCCAGU |
| F34 with 3 U | 103 | CGCCAGCCUCAAGTGTUGTCATGU |
| R34 with 3 U | 104 | CGCTCAAGUCGCATCCUTCGTCAU |
| F36 with 3 U | 105 | CATAAGCAUCGCCGCAUCGTACCU |
| R36 with 3 U | 106 | GCCGTCTGUGCCGAGAUAACCAGU |
| F45 with 3 U | 107 | GCCGAACCUCCGTGAAUCTGACCU |
| R45 with 3 U | 108 | GACGCTAAUCTCCGCCUGTGACCU |
| F47 with 3 U | 109 | GTCAATCGUCAGTCCAUCACGCCU |
| R47 with 3 U | 110 | CCAGTCAGUGCCGTCAUCAGCCTU |
| F52 with 3 U | 111 | ATGAGCAGUCTAACCTUGCCGCCU |
| R52 with 3 U | 112 | TCAGCACAUCATCACCUGCGTTGU |
| F53 with 3 U | 113 | ACCGTAAGUCCGTCTGUCACCGAU |
| R53 with 3 U | 114 | GCTGGCGTUGAGTCCAUTAACCGU |
| F61 with 4 U | 115 | CGCCGAUAGTGTUGCCGAUCCGAU |
| R61 with 4 U | 116 | TCGAAGUGCCATUCCGCCUGACCU |
| F66 with 4 U | 117 | GCAGCGUCACCAUCTTGCUCACCU |
| R66 with 4 U | 118 | GCAGCGUCCACAUCCAACUCCGTU |
| F67 with 4 U | 119 | CGAGCCUGCCATUCACCTUGCCAU |
| R67 with 4 U | 120 | AAGTGAUCCGTGUCACCGUTGCGU |
| F73 with 4 U | 121 | CATCCGUGACCGUGCCGAUTGAGU |
| R73 with 4 U | 122 | ACAACCUTGCCGUGCCAGUGAGTU |
| F75 with 4 U | 123 | GCACGCUCGTCCUCACAGUCACTU |
| R75 with 4 U | 124 | CGCAGGUCACCAUTCTCAUCGCCU |
| F84 with 4 U | 125 | CGCCAGUCCGCTUCCATCUGACAU |
| R84 with 4 U | 126 | GCTCGCUCCGCCUTGAAGUAACCU |
| F89 with 4 U | 127 | AGCCGAUTCCGTUCCACCUTGCAU |
| R89 with 4 U | 128 | GTCCACUGAGCCUCCACCUAGCCU |
| F95 with 4 U | 129 | TGCCAAUCATGCUCCACCUTGCGU |
| R95 with 4 U | 130 | GCAGCCUACACCUTGCCTUACCGU |

Table 22. Universal primers for target nucleic acid amplification.

Amplification with Universal Primers

A double-stranded target nucleic acid (180 bp) was PCR amplified using each of the pairs of universal primers listed in Table 22. The amplification reaction was prepared using the reagents as shown in Table 23. The amplification cycles were performed according to the sequence shown in Table 24.

TABLE 23

| Reaction Components | Volume (μL) | Final Concentration |
|---|---|---|
| 2x KAPA Uracil + ready mix | 100 | 1x |
| Target DNA (50 pg/μl) | 4 | 1 pg/μl |
| Universal forward primer (20 μM) | 3 | 0.3 uM |
| Universal reverse primer (20 μM) | 3 | 0.3 uM |
| Water | 90 | |

Table 23. PCR reaction mixture using universal primers.

TABLE 24

| Step | Temperature (° C.) | Time (seconds) |
|---|---|---|
| Initial Denaturation | 95 | 45 |
| 25 Cycles | 98 | 10 |
| | 66, 71 or 72 | 15 |
| | (primer dependent) | |
| | 72 | 12 |
| Final Extension | 72 | 30 |

Table 24. Thermocycling conditions for PCR.

The PCR reaction products comprised double-stranded amplicons comprising copies of the target nucleic acid, with each strand of the double-stranded amplicons further comprising a sequence from the forward universal primer or the second universal primer extended during the PCR reaction. The amplicons were purified with Promega PCR Clean-UP System (e.g., Wizard®), and then quantified.

Removal of Universal Primer Sequence from Amplicons

To remove uracil nucleobases from the forward and reverse universal primer sequences of the double-stranded amplicons, the double-stranded amplicons were combined with in a cleavage reaction comprising endonuclease VIII (Endo VIII) and uracil DNA glycosylase (UDG). The cleavage reaction components are shown in Table 25. 10× CutSmart® was obtained from New England BioLabs (Product # B7204S). UDG and Endo VIII were obtained from Enzymatics (Product # Y9180L). The cleavage reactions occurred at 37° C. for 1 hour.

TABLE 25

| Cleavage Reaction Components | Volume (μL) |
|---|---|
| PCR amplicon (37-89 ng/μl) | 16 |
| 10x CutSmart ® Buffer | 2 |
| EndoVIII | 1 |
| UDG | 1 |

Table 25. PCR amplicon cleavage reaction mixture.

Products of the cleavage reaction comprised copies of the target nucleic acid and non-uracil nucleobases remaining from the extended universal primers. Removal of non-target nucleic acid sequence was achieved by combining the products of the cleavage reaction with an equal volume (20 μL) of PCR mixture A (8 μL 5×KAPA buffer, 0.8 μL of 10 mM dTNPs, 0.8 μL KAPA HiFi at 1 U/μL, 10.4 μL water), with incubation at 70° C. for 1 hour. The resulting products were resolved on a DNA agarose gel and removal of universal primer sequence was evaluated by analysis of BioA traces from the DNA agarose gel.

Primer Sequence Removal Efficiency

The yield of target nucleic acid amplicons after universal primer sequence removal was lower with universal primers comprising one or two uracil nucleobases as compared to universal primers comprising three or four uracil nucleobases. Select target nucleic acid amplicons were cloned into a TOPO vector and sequenced using next generation sequencing. The removal efficiency correlating with universal primer sequence is shown in Table 26. Removal efficiency was calculated by the equation: 100*(perfect removals)/(imperfect removals), where perfect removals is the number of instances where no primer sequence was identified between the Topo vector and the 5′ terminal nucleobase of the target nucleic acid, and where imperfect removals is the number of instances where Topo vector sequence was separated by a 5′ or 3′ terminal nucleobase of the target nucleic acid with a primer sequence. Universal primers having three or four nucleobases spaced apart by three to ten non-uracil nucleobases had universal primer sequence removal efficiencies in resulting amplicons greater than 99%. Universal primer pairs in rows 1 to 3 in Table 26 had removal efficiencies from their amplified target nucleic acid of 100%.

TABLE 26

| | | SEQ | | |
|---|---|---|---|---|
| | Primer from | ID | | Removal |
| | Primer Pair | NO. | Sequence | Efficiency (%) |
| 1 | R16 with 2 U | 96 | GCCGCGTTATUCATACAGCCGU | 100.000 |
| 2 | R52 with 3 U | 112 | TCAGCACAUCATCACCUGCGTTGU | 100.000 |
| 3 | R28 with 3 U | 100 | GGTCAATCUGCCGCAAUCCAGTCU | 100.000 |
| 4 | R10 with 2 U | 92 | ACACTCAGCGUTCCGTTACCGU | 99.999 |
| 5 | R47 with 3 U | 110 | CCAGTCAGUGCCGTCAUCAGCCTU | 99.992 |
| 6 | F04 with 1 U | 85 | AAGCCAGACCGTTCCACAAU | 99.992 |
| 7 | R61 with 4 U | 116 | TCGAAGUGCCATUCCGCCUGACCU | 99.971 |
| 8 | R53 with 3 U | 114 | GCTGGCGTUGAGTCCAUTAACCGU | 99.966 |
| 9 | R67 with 4 U | 120 | AAGTGAUCCGTGUCACCGUTGCGU | 99.923 |
| 10 | R36 with 3 U | 106 | GCCGTCTGUGCCGAGAUAACCAGU | 99.664 |
| 11 | F16 with 2U | 95 | CCAGCCAGTCUCCGTCAGTCAU | 98.530 |
| 12 | F10 with 2U | 91 | AAGCTCAGCCUCCAGTTGCTCU | 97.833 |

Table 26. Primer sequence removal efficiency.

### Example 8: Selection of Uracil Nucleobase Number and Position in Universal Primers

Universal primer pairs were designed having three or four uracil nucleobases separated within each universal primer by five to eight non-uracil nucleobases. Each of the plurality of universal primers was used to amplify a target nucleic acid flanked with adapter sequences by PCR; wherein each of the plurality of universal primers bound to an adapter sequence, followed by extension into amplicons of the target nucleic acid. PCR reaction conditions were optimized to increase universal primer and adapter sequence removal efficiency, while decreasing incidence of 3′ uracil nucleobase removal from universal primers by DNA polymerase during amplification.

Preparation of a Target Nucleic Acid for Universal Primer Amplification

Target nucleic acids were amplified with primer pairs comprising an adapter sequence that differs from a universal primer sequence by replacing the uracil nucleobases from the universal primer sequence with a thymidine nucleobase in the adapter sequence. Adapter and corresponding universal primer sequences are shown in Table 27. Amplification was performed using amplification reagents combined as shown in Table 28. Amplification was performed by PCR using the cycles shown in Table 29.

TABLE 27

| | SEQ | | SEQ | |
|---|---|---|---|---|
| Primer | ID | Adapter Primer | ID | Universal Primer |
| Name | NO. | Sequence | NO. | Sequence |
| prm2_0 | 131 | CGCCACGGTTCCTTATCG | 227 | CGCCACGGUTCCTTAU |
| 1F | | CAGTT | | CGCAGTU |
| prm2_0 | 132 | ACCCACCCTCAGCAGTTC | 228 | ACCCACCCUCAGCAGU |
| 2F | | ACGAT | | TCACGAU |
| prm2_0 | 133 | GCTCTGACTGCGCCATAG | 229 | GCTCTGACUGCGCCAU |
| 3F | | ACTGT | | AGACTGU |
| prm2_0 | 134 | TCACCGCCTCTCCTTTGA | 230 | TCACCGCCUCTCCTTU |
| 4F | | CCAGT | | GACCAGU |
| prm2_0 | 135 | TCACCCAGTCAGCCGTTT | 231 | TCACCCAGUCAGCCGU |
| 5F | | ACAGT | | TTACAGU |
| prm2_0 | 136 | CGTTCGCCTTGCCTGTAG | 232 | CGTTCGCCUTGCCTGU |
| 6F | | ACACT | | AGACACU |
| prm2_0 | 137 | AGCTGCGGTCTGATGTCA | 233 | AGCTGCGGUCTGATGU |
| 7F | | CTGAT | | CACTGAU |
| prm2_0 | 138 | CCCCCATATCGCCGTTCA | 234 | CCCCCATAUCGCCGTU |
| 8F | | GCTAT | | CAGCTAU |
| prm2_0 | 139 | TCTCCCCATCGCCAGTCA | 235 | TCTCCCCAUCGCCAGU |
| 9F | | GTCAT | | CAGTCAU |
| prm2_1 | 140 | GCCCGCCCTTTTCAATGT | 236 | GCCCGCCCUTTTCAAU |
| 0F | | GTCAT | | GTGTCAU |
| prm2_1 | 141 | CGCCGTCCTAAGCCCTGT | 237 | CGCCGTCCUAAGCCCU |
| 1F | | ATGAT | | GTATGAU |
| prm2_1 | 142 | CGACGCCGTTGACCATAG | 238 | CGACGCCGUTGACCAU |
| 2F | | ATGCT | | AGATGCU |
| prm2_1 | 143 | CCGATTCCTCTCCCGTGC | 239 | CCGATTCCUCTCCCGU |
| 3F | | AGTAT | | GCAGTAU |
| prm2_1 | 144 | GCCGCACTTTGAGAATGA | 240 | GCCGCACTUTGAGAAU |
| 4F | | CGCTT | | GACGCTU |
| prm2_1 | 145 | GCAGCCCCTAGTCAGTCG | 241 | GCAGCCCCUAGTCAGU |
| 5F | | CTAAT | | CGCTAAU |
| prm2_1 | 146 | AACCTGTGTTCCGCCTGA | 242 | AACCTGTGUTCCGCCU |
| 6F | | GTGAT | | GAGTGAU |
| prm2_1 | 147 | CCCGAGAGTGTGTGATGC | 243 | CCCGAGAGUGTGTGAU |
| 7F | | CGTAT | | GCCGTAU |
| prm2_1 | 148 | TGGACCCTTGCCAGATGA | 244 | TGGACCCTUGCCAGAU |
| 8F | | GCCTT | | GAGCCTU |
| prm2_1 | 149 | CGCACCCTTCCCTAGTAA | 245 | CGCACCCTUCCCTAGU |
| 9F | | TCCGT | | AATCCGU |
| prm2_2 | 150 | GCCCACCCTACAGAGTTG | 246 | GCCCACCCUACAGAGU |
| 0F | | ACCCT | | TGACCCU |
| prm2_2 | 151 | CCCCCCTTTACCCCTTGCC | 247 | CCCCCCTTUACCCCTU |
| 1F | | AGAT | | GCCAGAU |
| prm2_2 | 152 | GACCCTTCTGTGCCTTGA | 248 | GACCCTTCUGTGCCTU |
| 2F | | CGACT | | GACGACU |
| prm2_2 | 153 | TCGTCCACTGTCCCCTAA | 249 | TCGTCCACUGTCCCCU |
| 3F | | TGGCT | | AATGGCU |
| prm2_2 | 154 | CCCGACTATCCTGCGTGA | 250 | CCCGACTAUCCTGCGU |
| 4F | | ACCAT | | GAACCAU |
| prm2_2 | 155 | TCAACCCCTGTAAGATGG | 251 | TCAACCCCUGTAAGAU |
| 5F | | CCCGT | | GGCCCGU |
| prm2_2 | 156 | CCACTGGTTGTTCACTGC | 252 | CCACTGGTUGTTCACU |
| 6F | | TCGGT | | GCTCGGU |
| prm2_2 | 157 | TGACACTGTACACGATTG | 253 | TGACACTGUACACGAU |
| 7F | | GCCCT | | TGGCCCU |
| prm2_2 | 158 | GGTTACTCTCTCGGCTCG | 254 | GGTTACTCUCTCGGCU |
| 8F | | CGTAT | | CGCGTAU |
| prm2_2 | 159 | TGAGGGTTTCATCCGTGT | 255 | TGAGGGTTUCATCCGU |
| 9F | | CGCAT | | GTCGCAU |
| prm2_3 | 160 | CCCCTATGTCATGAGTGC | 256 | CCCCTATGUCATGAGU |
| 0F | | CCGAT | | GCCCGAU |
| prm2_3 | 161 | TGCTATGCGTGTCCATGT | 257 | TGCTAUGCGTGUCCAT |
| 1F | | ACCGTT | | GUACCGTU |
| prm2_3 | 162 | GCCAATCCGTGTCCGTGT | 258 | GCCAAUCCGTGUCCGT |
| 2F | | AACTTT | | GUAACTTU |
| prm2_3 | 163 | AACGCTTGGGGTGGAACT | 259 | AACGCUTGGGGUGGAA |
| 3F | | CTTAGT | | CUCTTAGU |
| prm2_3 | 164 | GTCGATGCCCCTCCAGCT | 260 | GTCGAUGCCCCUCCAG |
| 4F | | ATGAAT | | CUATGAAU |
| prm2_3 | 165 | CCCCGTCACCTTTGGCTT | 261 | CCCCGUCACCTUTGGC |
| 5F | | ATCAGT | | TUATCAGU |
| prm2_3 | 166 | GCCCCTTCGACTGAACTT | 262 | GCCCCUTCGACUGAAC |
| 6F | | TACGGT | | TUTACGGU |
| prm2_3 | 167 | GGCCCTGAGTATGGACCT | 263 | GGCCCUGAGTAUGGAC |
| 7F | | AGCTGT | | CUAGCTGU |
| prm2_3 | 168 | TGCCGTCCAAGTCTCAGT | 264 | TGCCGUCCAAGUCTCA |
| 8F | | CTCAGT | | GUCTCAGU |
| prm2_3 | 169 | GCAGGTTATCATCCCAGT | 265 | GCAGGUTATCAUCCCA |
| 9F | | GACGCT | | GUGACGCU |
| prm2_4 | 170 | CGGTGTCATGGTGAAGAT | 266 | CGGTGUCATGGUGAAG |
| 0F | | CAGGCT | | AUCAGGCU |
| prm2_4 | 171 | GCCAGTTGCCATCAGAAT | 267 | GCCAGUTGCCAUCAGA |
| 1F | | CCCAGT | | AUCCCAGU |
| prm2_4 | 172 | ACGTTTCGCACTCTTGGT | 268 | ACGTTUCGCACUCTTG |
| 2F | | AGCACT | | GUAGCACU |
| prm2_4 | 173 | ATGTGTGTAGGTCAAGAT | 269 | ATGTGUGTAGGUCAAG |
| 3F | | GCCCGT | | AUGCCCGU |
| prm2_4 | 174 | CCCCATTCGTTTCAGCGT | 270 | CCCCAUTCGTTUCAGC |
| 4F | | ACCAGT | | GUACCAGU |
| prm2_4 | 175 | CTC CCTTAGGTTTGGCAT | 271 | CTCCCUTAGGTUTGGC |
| 5F | | CGCAGT | | AUCGCAGU |
| prm2_4 | 176 | CGCAGTTTCCCTTTAGAT | 272 | CGCAGUTTCCCUTTAG |
| 6F | | CGCCGT | | AUCGCCGU |
| prm2_4 | 177 | CTTGTTGAGCCTGTGAGT | 273 | CTTGTUGAGCCUGTGA |
| 7F | | GACCCT | | GUGACCCU |
| prm2_4 | 178 | TTACCTTACCCTCGGACT | 274 | TTACCUTACCCUCGGA |
| 8F | | CAGCGT | | CUCAGCGU |
| prm2_0 | 179 | CGCCCCCGTTCCTAATCA | 275 | CGCCCCCGUTCCTAAU |
| 1R | | GTTCT | | CAGTTCU |
| prm2_0 | 180 | GCCAGTCCTCCTCAGTTT | 276 | GCCAGTCCUCCTCAGU |
| 2R | | ACCGT | | TTACCGU |
| prm2_0 | 181 | CCTGCCCGTATCGCCTGT | 277 | CCTGCCCGUATCGCCU |
| 3R | | AAGTT | | GTAAGTU |
| prm2_0 | 182 | ACGCCGCCTGTTATATGA | 278 | ACGCCGCCUGTTATAU |
| 4R | | AGCCT | | GAAGCCU |
| prm2_0 | 183 | CGCCAGCCTTGTCAGTCT | 279 | CGCCAGCCUTGTCAGU |
| 5R | | TGCAT | | CTTGCAU |
| prm2_0 | 184 | GCCGACCCTCTGAATTAT | 280 | GCCGACCCUCTGAATU |
| 6R | | GCCGT | | ATGCCGU |
| prm2_0 | 185 | AGGGGGTTTGTGCCATTG | 281 | AGGGGGTTUGTGCCAU |
| 7R | | TCGAT | | TGTCGAU |
| prm2_0 | 186 | AGCTTGCCTCGTCAGTCA | 282 | AGCTTGCCUCGTCAGU |
| 8R | | GTCTT | | CAGTCTU |
| prm2_0 | 187 | CGCACCCGTCAACAATCG | 283 | CGCACCCGUCAACAAU |
| 9R | | CTTAT | | CGCTTAU |
| prm2_1 | 188 | TGTGCCAGTCCCGAATCC | 284 | TGTGCCAGUCCCGAAU |
| 0R | | AGAGT | | CCAGAGU |
| prm2_1 | 189 | GACCGCTCTCCCAGATTG | 285 | GACCGCTCUCCCAGAU |
| 1R | | ACACT | | TGACACU |
| prm2_1 | 190 | ACCGACCCTTTCCGATGT | 286 | ACCGACCCUTTCCGAU |
| 2R | | TCCAT | | GTTCCAU |
| prm2_1 | 191 | CCTGATGCTCCCGTGTGA | 287 | CCTGATGCUCCCGTGU |
| 3R | | CAACT | | GACAACU |
| prm2_1 | 192 | TGGGACGCTGCTAGGTAG | 288 | TGGGACGCUGCTAGGU |
| 4R | | ACACT | | AGACACU |
| prm2_1 | 193 | GCAGACCGTACCTTGTAG | 289 | GCAGACCGUACCTTGU |
| 5R | | ACCGT | | AGACCGU |
| prm2_1 | 194 | AGACACCCTCCGAAGTTG | 290 | AGACACCCUCCGAAGU |
| 6R | | CCGAT | | TGCCGAU |
| prm2_1 | 195 | ACGTAGCTTCCCCCCTGA | 291 | ACGTAGCTUCCCCCCU |
| 7R | | TGAGT | | GATGAGU |
| prm2_1 | 196 | GAGGACTTTGCCCCGTGA | 292 | GAGGACTTUGCCCCGU |
| 8R | | GACTT | | GAGACTU |
| prm2_1 | 197 | GCCACTCATTGACAATAC | 293 | GCCACTCAUTGACAAU |
| 9R | | GCCGT | | ACGCCGU |
| prm2_2 | 198 | GTTCACAGTCCCCAATGA | 294 | GTTCACAGUCCCCAAU |
| 0R | | GCCCT | | GAGCCCU |
| prm2_2 | 199 | CCCAGGACTTGTGAATTG | 295 | CCCAGGACUTGTGAAU |
| 1R | | CCCGT | | TGCCCGU |
| prm2_2 | 200 | GGGGGCGTTTACTGATAC | 296 | GGGGGCGTUTACTGAU |
| 2R | | CGACT | | ACCGACU |
| prm2_2 | 201 | ACTGCACATCCCTCGTGA | 297 | ACTGCACAUCCCTCGU |
| 3R | | ACCTT | | GAACCTU |
| prm2_2 | 202 | ATGCCTCCTCCTGACTGA | 298 | ATGCCTCCUCCTGACU |
| 4R | | CCGTT | | GACCGTU |
| prm2_2 | 203 | CAACGTGCTTTCCGATCC | 299 | CAACGTGCUTTCCGAU |
| 5R | | CGTGT | | CCCGTGU |
| prm2_2 | 204 | GGGGTAGGTCACTGGTCG | 300 | GGGGTAGGUCACTGGU |
| 6R | | CTCAT | | CGCTCAU |
| prm2_2 | 205 | CTGTTGTATCCCACCTGA | 301 | CTGTTGTAUCCCACCU |
| 7R | | CGGCT | | GACGGCU |
| prm2_2 | 206 | CGTCTACATCCAAGGTCC | 302 | CGTCTACAUCCAAGGU |
| 8R | | GCACT | | CCGCACU |
| prm2_2 | 207 | GGATCGTGTGTGTAGTCC | 303 | GGATCGTGUGTGTAGU |
| 9R | | AGCGT | | CCAGCGU |
| prm2_3 | 208 | GTGTAGAATGACCAGTGC | 304 | GTGTAGAAUGACCAGU |
| 0R | | CCCGT | | GCCCCGU |
| prm2_3 | 209 | AGCCGTCGTCCTCTAAGT | 305 | AGCCGUCGTCCUCTAA |
| 1R | | CACTGT | | GUCACTGU |
| prm2_3 | 210 | AGTGCTGAACCTCCTTGT | 306 | AGTGCUGAACCUCCTT |
| 2R | | GAACCT | | GUGAACCU |
| prm2_3 | 211 | GGACTTTGGGCTCGGACT | 307 | GGACTUTGGGCUCGGA |
| 3R | | CTTGAT | | CUCTTGAU |
| prm2_3 | 212 | ATCCCTTTGCCTGCCTATC | 308 | ATCCCUTTGCCUGCCT |
| 4R | | GGAAT | | AUCGGAAU |
| prm2_3 | 213 | CGGTCTTAGCGTCCAGAT | 309 | CGGTCUTAGCGUCCAG |
| 5R | | GTCACT | | AUGTCACU |
| prm2_3 | 214 | CCGGCTCCCATTTGATTTT | 310 | CCGGCUCCCATUTGAT |
| 6R | | GCGAT | | TUTGCGAU |
| prm2_3 | 215 | CCCCGTAGTGTTACCGTT | 311 | CCCCGUAGTGTUACCG |
| 7R | | CCGAAT | | TUCCGAAU |
| prm2_3 | 216 | ACCGCTCTCCATATCAAT | 312 | ACCGCUCTCCAUATCA |
| 8R | | CGCAGT | | AUCGCAGU |
| prm2_3 | 217 | GGCATTCCCTCTCTTCGT | 313 | GGCATUCCCTCUCTTC |
| 9R | | AGCCAT | | GUAGCCAU |
| prm2_4 | 218 | CCCCCTACTCGTTACACT | 314 | CCCCCUACTCGUTACA |
| 0R | | GGCATT | | CUGGCATU |
| prm2_4 | 219 | GCTTGTCCCTTTCCCAATC | 315 | GCTTGUCCCTTUCCCA |
| 1R | | CGAGT | | AUCCGAGU |
| prm2_4 | 220 | AGCCGTTCTTGTTCCTGTC | 316 | AGCCGUTCTTGUTCCT |
| 2R | | GCAAT | | GUCGCAAU |
| prm2_4 | 221 | CACCCTCTTAGTCCCAGT | 317 | CACCCUCTTAGUCCCA |
| 3R | | CCAGGT | | GUCCAGGU |
| prm2_4 | 222 | CATAGTCGCAGTTCCCGT | 318 | CATAGUCGCAGUTCCC |
| 4R | | CAGAGT | | GUCAGAGU |
| prm2_4 | 223 | ATTGGTCTCGCTCTCAGT | 319 | ATTGGUCTCGCUCTCA |
| 5R | | CAGGCT | | GUCAGGCU |
| prm2_4 | 224 | ATGACTGGCTTTGACCGT | 320 | ATGACUGGCTTUGACC |
| 6R | | GGACAT | | GUGGACAU |
| prm2_4 | 225 | AAGCCTGCCGATATGCCT | 321 | AAGCCUGCCGAUATGC |
| 7R | | GAGACT | | CUGAGACU |
| prm2_4 | 226 | GCACATCATCGTCAGGCT | 322 | GCACAUCATCGUCAGG |
| 8R | | CACAGT | | CUCACAGU |

Table 27. Universal and adapter primers for target nucleic acid amplification.

TABLE 28

| Reaction Components | Volume (μL) | Final Concentration |
|---|---|---|
| Q5 DNA Polymerase (2 U/μL) | 0.5 | 0.2 U/μL |
| 5x Q5 Buffer | 10 | 1x |
| Target DNA (50 pg/μl) | 1 | 1 pg/μl |
| Forward adapter primer (20 μM) | 1.25 | 0.5 μM |
| Reverse adapter primer (20 μM) | 1.25 | 0.5 μM |
| dNTP (10 mM) | 1 | 200 μM |
| Water | 35 | |

Table 28. PCR reaction mixture using adapter primers.

TABLE 29

| Step | Temperature (° C.) | Time (seconds) |
|---|---|---|
| Initial Denaturation | 98 | 30 |
| 25 Cycles | 98 | 10 |
| | 70 | 10 |
| | 72 | 10 |
| Final Extension | 72 | 300 |

Table 29. Thermocycling conditions for PCR with adapter primers.

The amplification products comprised: a) a first strand comprising a forward adapter primer sequence, a target nucleic acid sequence, and a reverse adapter primer sequence complement; and b) a second strand comprising a reverse adapter primer sequence, a target nucleic acid sequence complement, and a forward adapter primer sequence complement. The first adapter primer sequence complement is a binding site for the universal primer sharing sequence with the first adapter sequence, and the second adapter primer sequence complement is a binding site for the universal primer sharing sequence with the second adapter sequence. The amplification products were purified using Ampure XP beads following the manufacturer's protocol.

Amplification with Universal Primers

The purified amplification products generated using adapter primers were the templates for another round of PCR amplification using universal primers corresponding in sequence to the adapter primers. The universal primers are shown in Table 27. The amplification reaction was prepared using the reagents as shown in Table 30. The amplification cycles were performed according to the sequence shown in Table 31.

TABLE 30

| | Volume | Final |
|---|---|---|
| Reaction Components | (μL) | Concentration |
| Phusion U HS DNA Polymerase (2 U/μ,L) | 1 | 0.2 U/μL |
| 5× Phusion HF Buffer | 20 | 1× |
| Template DNA with adapter sequence | 2 | 1 pg/μl |
| (10×-200× dilution with TE buffer) | | |
| Universal forward primer (20 μM) | 1.5 | 0.3 μM |
| Universal reverse primer (20 μM) | 1.5 | 0.3 μM |
| dNTP (10 mM) | 2 | 200 μM |
| Water | 72 | |

Table 30. PCR reaction mixture using universal primers.

TABLE 31

| Step | Temperature (° C.) | Time (seconds) |
|---|---|---|
| Initial Denaturation | 98 | 30 |
| 22 Cycles | 98 | 10 |
| | 72 | 20 |
| Final Extension | 72 | 300 |

Table 31. Thermocycling conditions for PCR with universal primers.

The universal PCR reaction products comprised double-stranded amplicons comprising a) a first strand comprising the forward adapter primer sequence, target nucleic acid sequence, and reverse universal primer sequence; and b) a second strand comprising the reverse adapter primer sequence, the target nucleic acid sequence complement, and the forward universal primer sequence. A sample of each universal PCR reaction product was separated by gel electrophoresis and imaged using a BioA Analyzer. Remaining universal PCR reaction products were purified using Ampure XP beads.

Removal of Universal Primer Sequence from Amplicons

To remove uracil nucleobases from the forward and reverse universal primer sequences of the universal PCR reaction products, the universal PCR reaction products were combined with in a cleavage reaction comprising a combination of endonuclease VIII (Endo VIII) and uracil DNA glycosylase (UDG) in USER by New England Biolabs. The cleavage reaction components are shown in Table 32. The cleavage reactions occurred at 37° C. for 1.5 hours.

TABLE 32

| Cleavage Reaction Components | Volume (μL) |
|---|---|
| PCR amplicon | 16 |
| 10× CutSmart ® Buffer | 2 |
| USER | 2 |

Table 32. Cleavage reaction mixture for removing uracil nucleobases from universal PCR reaction products.

Products of the cleavage reaction had a plurality of gaps where the uracil nucleobases from each universal PCR reaction product was removed. To remove the remaining nucleobases of forward adapter primer sequence, reverse universal primer sequence, reverse adapter primer sequence, and forward universal primer sequence, the products were combined with 16 μL of PCR mixture B (4 μL 5×KAPA buffer, 0.8 μL of 10 mM dTNPs, 0.8 μL KAPA HiFi at 1 U/μL, and 10.4 μL water), with incubation at 75° C. for 1 hour. The resulting products were resolved on a DNA agarose gel and removal of universal primer sequence was evaluated by analysis of BioA traces from the DNA agarose gel.

Primer Sequence Removal Efficiency

The BioA traces were analyzed and peaks corresponding to DNA fragments resolved on the DNA agarose gel were integrated. To evaluate removal of universal primer and adapter sequences, the ratio of integrated target peak to side peaks was calculated. The results are shown in Table 33. Primer pairs having the highest ratios (from 96 to 100) were prm2_12, prm2_14, prm2_23, prm2_32, and prm2_35—each of which comprise three or four uracil nucleobases spaced apart by five to eight non-uracil nucleobases. In comparison Uni7 and Uni9 control primers having a single uracil nucleobase at the 3′ terminal end, had a ratio of 88.15.

TABLE 33

| Primer | Uracil | GC | Goal Peak | Side Peak | Ratio |
|---|---|---|---|---|---|
| Name | No. | content | (molarity) | (molarity) | (completion) |
| prm2_01 | 3 | 40 | 14.4 | 0.6 | 96.00 |
| prm2_02 | 3 | | 23.9 | 1 | 95.98 |
| prm2_03 | 3 | | 33 | 2.1 | 94.02 |
| prm2_04 | 3 | | 32.3 | 1.2 | 96.42 |
| prm2_05 | 3 | | 28.5 | 1.1 | 96.28 |
| prm2_06 | 3 | | 34.8 | 2.2 | 94.05 |
| prm2_07 | 3 | | 59.6 | 6.4 | 90.30 |
| prm2_08 | 3 | | 72 | 3.9 | 94.86 |
| prm2_09 | 3 | | 26.9 | 1.2 | 95.73 |
| prm2_10 | 3 | | 24.9 | 1.4 | 94.68 |
| prm2_11 | 3 | | 83 | 4.2 | 95.18 |
| prm2_12 | 3 | | 23.7 | 0.8 | 96.73 |
| prm2_13 | 3 | 50 | 36.2 | 1.7 | 95.51 |
| prm2_14 | 3 | | 51.3 | 0 | 100.00 |
| prm2_15 | 3 | | 48.4 | 2 | 96.03 |
| prm2_16 | 3 | | 57.4 | 4 | 93.49 |
| prm2_17 | 3 | | 33.3 | 2.1 | 94.07 |
| prm2_18 | 3 | | 0 | 13 | 0.00 |
| prm2_19 | 3 | | 11.4 | 3.8 | 75.00 |
| prm2_20 | 3 | | 29.3 | 1.2 | 96.07 |
| prm2_21 | 3 | | 26.9 | 2.2 | 92.44 |
| prm2_22 | 3 | | 3.2 | 1.2 | 72.73 |
| prm2_23 | 3 | | 34.3 | 1.1 | 96.89 |
| prm2_24 | 3 | | 40.4 | 1.5 | 96.42 |
| prm2_25 | 3 | 60 | 70.1 | 6.3 | 91.75 |
| prm2_26 | 3 | | 47.5 | 3.4 | 93.32 |
| prm2_27 | 3 | | 40.6 | 1.9 | 95.53 |
| prm2_28 | 3 | | 39.3 | 2.2 | 94.70 |
| prm2_29 | 3 | | 72.2 | 3.9 | 94.88 |
| prm2_30 | 3 | | 51 | 2.1 | 96.05 |
| prm2_31 | 4 | 40 | 34.1 | 1.7 | 95.25 |
| prm2_32 | 4 | | 31.7 | 0 | 100.00 |
| prm2_33 | 4 | | 34 | 2.9 | 92.14 |
| prm2_34 | 4 | | 36.2 | 5 | 87.86 |
| prm2_35 | 4 | | 80 | 0 | 100.00 |
| prm2_36 | 4 | | 27 | 1.6 | 94.41 |
| prm2_37 | 4 | 50 | 33.4 | 5.4 | 86.08 |
| prm2_38 | 4 | | 40 | 5 | 88.89 |
| prm2_39 | 4 | | 34.2 | 3 | 91.94 |
| prm2_40 | 4 | | 22.8 | 3.1 | 88.03 |
| prm2_41 | 4 | | 45.9 | 4.6 | 90.89 |
| prm2_42 | 4 | | 24.1 | 1.8 | 93.05 |
| prm2_43 | 4 | 60 | 50.8 | 4.6 | 91.70 |
| prm2_44 | 4 | | 57.4 | 3.9 | 93.64 |
| prm2_45 | 4 | | 45.7 | 3.3 | 93.27 |
| prm2_46 | 4 | | 79.9 | 4 | 95.23 |
| prm2_47 | 4 | | 97.3 | 6.7 | 93.56 |
| prm2_48 | 4 | | 103.7 | 9.3 | 91.77 |
| Uni7 ctrl | 1 | | 11.9 | 1.6 | 88.15 |
| Uni9 ctrl | 1 | | 24.2 | 1.5 | 94.16 |

Table 33. Analysis of DNA fragments separated by gel electrophoresis.

While specific embodiments have been shown and described herein, it will be apparent to those skilled in the art that such embodiments are provided by way of example only. Numerous variations, changes, and substitutions will now occur to those skilled in the art without departing from the disclosed embodiments. It should be understood that various alternatives to the embodiments described herein may be employed.

What is claimed is:

1. A method for nucleic acid amplification comprising:
a) providing a double stranded template nucleic acid comprising a first strand and a second strand;
b) mixing the double stranded template nucleic acid with a first primer and a second primer, wherein:
i) the first primer comprises a first plurality of nucleobases that are not canonical DNA nucleobases, wherein each nucleobase of the first plurality of nucleobases that are not canonical DNA nucleobases is distanced from another by about 3 to about 8 nucleobases, and wherein one nucleobase of the first plurality of nucleobases that are not canonical DNA nucleobases is a 3′ terminal nucleobase bound to a preceding nucleobase in the first primer by a phosphorothioate bond, and
ii) the second primer comprises a second plurality of nucleobases that are not canonical DNA nucleobases, wherein each nucleobase of the second plurality of nucleobases that are not canonical DNA nucleobases is distanced from another by about 3 to about 8 nucleobases, and wherein one nucleobase of the second plurality of nucleobases that are not canonical DNA nucleobases is a 3′ terminal nucleobase bound to a preceding nucleobase in the second primer by a phosphorothioate bond; and
c) amplifying the double stranded template nucleic acid by extending the first primer to form a first extension strand and extending the second primer to form a second extension strand; and
d) removing the first primer from the first extension strand and the second primer from the second extension strand.

2. The method of claim 1, wherein the first plurality of nucleobases that are not canonical DNA nucleobases or the second plurality of nucleobases that are not canonical DNA nucleobases comprises 3 or 4 nucleobases.

3. The method of claim 1, wherein each nucleobase of the first plurality of nucleobases that are not canonical DNA nucleobases is distanced from another by about 4 to about 7 nucleobases.

4. The method of claim 3, wherein each nucleobase of the second plurality of nucleobases that are not canonical DNA nucleobases is distanced from another by about 4 to about 7 nucleobases.

5. The method of claim 1, wherein the first primer and the second primer each have a total length of from about 12 to about 50 nucleobases.

6. The method of claim 1, wherein each nucleobase of the first plurality of nucleobases that are not canonical DNA nucleobases or each nucleobase of the second plurality of nucleobases that are not canonical DNA nucleobases is uracil, 3-meA (3-methyladenine), hypoxanthine, 8-oxoG (7,8-dihydro-8-oxoguanine), FapyG, FapyA, Tg (thymine glycol), hoU (hydroxyuracil), hmU (hydroxymethyluracil), fU (formyluracil), hoC (hydroxycytosine), fC (formylcytosine), 5-meC (5-methylcytosine), 6-meG (O6-methylguanine), 7-meG (N7-methylguanine), EC (ethenocytosine), 5-caC (5-carboxylcytosine), 2-hA, EA (ethenoadenine), 5-fU (5-fluorouracil), 3-meG (3-methylguanine), or isodialuric acid.

7. The method of claim 6, wherein the first plurality of nucleobases that are not canonical DNA nucleobases and the second plurality of nucleobases that are not canonical DNA nucleobases are the same type of nucleobase.

8. The method of claim 1, wherein each nucleobase of the first plurality of nucleobases that are not canonical DNA nucleobases and each nucleobase of the second plurality of nucleobases that are not canonical DNA nucleobases is uracil.

9. The method of claim 8, wherein the nucleobase preceding the 3′ terminal uracil nucleobase in the first primer is a pyrimidine.

10. The method of claim 8, wherein the nucleobase preceding the 3′ terminal uracil nucleobase in the second primer is a pyrimidine.

11. The method of claim 9 or 10, wherein the pyrimidine is a cytosine.

12. The method of claim 8, wherein the first primer is removed from the first extension strand with an efficiency of at least about 90%, and the second primer is removed from the second extension strand with an efficiency of at least about 90%.

13. The method of claim 1, wherein the melting temperature of the first primer is between about 60° C. and about 66° C.

14. The method of claim 1, wherein the melting temperature of the second primer is between about 60° C. and about 66° C.

15. The method of claim 1, wherein the GC content of the first primer, the second primer, or both the first primer and the second primer is between about 40% and about 60%.

16. The method of claim 8, wherein removing the first primer from the first extension strand comprises excising the first plurality of uracil nucleobases from the first primer from the first extension strand, and wherein removing the second primer from the second extension strand comprises excising the second plurality of uracil nucleobases from the second primer from the second extension strand.

17. The method of claim 16, wherein excision of the first plurality of uracil nucleobases from the first primer of the first extension strand and excision of the second plurality of uracil nucleobases from the second primer of the second extension strand is achieved by treating the first extension strand and the second extension strand with a mixture of Uracil DNA glycosylase and a DNA glycosylase-lyase Endonuclease VIII.

18. The method of claim 8, wherein the first strand of the double stranded template nucleic acid comprises in 5′ to 3′ order: a first primer binding site complementary to the first primer, and a target nucleic acid sequence; and the second strand of the double stranded template nucleic acid comprises in 5′ to 3′ order: a second primer binding site complementary to the second primer, and a nucleic acid sequence complementary to the target nucleic acid sequence.

19. The method of claim 18, wherein the first strand of the double stranded template nucleic acid comprises in 5′ to 3′ order: the first primer binding site complementary to the first primer, the target nucleic acid sequence, and a nucleic acid sequence complementary to the second primer binding site; and the second strand of the double stranded template nucleic acid comprises in 5′ to 3′ order: the second primer binding site complementary to the second primer, the nucleic acid sequence complementary to the target nucleic acid sequence, and a nucleic acid sequence complementary to the first primer binding site.

20. The method of claim 19, wherein the first extension strand comprises in 5′ to 3′ order: the first primer, the target nucleic acid sequence, and a nucleic acid sequence complementary to the second primer; and the second extension strand comprises in 5′ to 3′ order: the second primer, the nucleic acid sequence complementary to the target nucleic acid sequence, and a nucleic acid sequence complementary to the first primer.

21. The method of claim 20, further comprising removing from the second extension strand the nucleic acid sequence complementary to the first primer and removing from the first extension strand the nucleic acid sequence complementary to the second primer.

22. The method of claim 21, wherein the nucleic acid sequence complementary to the first primer and the nucleic acid sequence complementary to the second primer are removed by digestion with an enzyme comprising exonuclease activity.

23. The method of claim 22, wherein the enzyme comprising exonuclease activity is a DNA polymerase.

24. The method of claim 1, wherein each strand of the double stranded templated nucleic acid is generated by assembly of de novo synthesis.

25. The method of claim 1, wherein each strand of the double stranded templated nucleic acid is generated by assembly of de novo synthesis, and wherein de novo synthesis comprises synthesis of a plurality of oligonucleotides which collectively encode for a sequence of the double stranded template nucleic acid.

26. The method of claim 1, wherein the double stranded target nucleic acid has a length from about 160 to about 10,000 nucleobases.

27. A method for amplifying a template nucleic acid, the method comprising:
a) providing a template nucleic acid comprising (i) a first template strand comprising in 5′ to 3′ order: a first primer binding site and a target nucleic acid, and (ii) a second template strand comprising in 5′ to 3′ order: a second primer binding site and a nucleic acid sequence complementary to the target nucleic acid;
b) annealing a first primer to the first primer binding site, and annealing a second primer to the second primer binding site, wherein:
the first primer comprises 3 or 4 uracil nucleobases, each uracil nucleobase distanced from another uracil nucleobase in the first primer by about 3 to about 8 non-uracil nucleobases,
the second primer comprises 3 or 4 uracil nucleobases, each uracil nucleobase distanced from another uracil nucleobase in the second primer by about 3 to about 8 non-uracil nucleobases,
one uracil nucleobase of the 3 or 4 uracil nucleobases in the first primer is a 3′ terminal uracil nucleobase bound to a preceding nucleobase in the first primer by a first nuclease resistant bond, and
one uracil nucleobase of the 3 or 4 uracil nucleobases in the second primer is a 3′ terminal uracil nucleobase bound to a preceding nucleobase in the second primer by a second nuclease resistant bond;
c) forming an amplicon nucleic acid by: extending the first primer and the second primer to generate a first extension strand comprising the first primer and the target nucleic acid, and extending the second primer to generate a second extension strand comprising the second primer and the nucleic acid sequence complementary to the target nucleic acid; and
d) removing the first primer and the second primer from the amplicon nucleic acid.

28. The method of claim 27, wherein the first nuclease resistant bond or the second nuclease resistant bond is a phosphorothioate bond.

29. The method of claim 27, wherein the first primer or the second primer comprises 3 uracil nucleobases.

30. The method of claim 27, wherein the first primer or the second primer comprises 4 uracil nucleobases.

31. The method of claim 27, wherein the nucleobase preceding the 3′ terminal uracil nucleobase in the first primer is a pyrimidine.

32. The method of claim 31, wherein the nucleobase preceding the 3′ terminal uracil nucleobase in the second primer is a pyrimidine.

33. The method of claim 27, wherein the first primer is removed from the first extension strand with an efficiency of at least about 90%, and the second primer is removed from the second extension strand with an efficiency of at least about 90%.

34. The method of claim 27, wherein removing the first primer and removing the second primer comprises: excising the 3 or 4 uracil nucleobases from the first primer of the first extension strand, and excising the 3 or 4 uracil nucleobases from the second primer of the second extension strand.

35. The method of claim 34, wherein excision of the 3 or 4 uracil nucleobases from the first primer of the first extension strand and excision of the 3 or 4 nucleobases from the second primer of the second extension strand is achieved by treating the first extension strand and the second extension strand with a mixture of Uracil DNA glycosylase and a DNA glycosylase-lyase Endonuclease VIII.

36. The method of claim 34, wherein the first template strand comprises in 5′ to 3′ order: the first primer binding site, the target nucleic acid, and a nucleic acid sequence complementary to the second primer binding site; and the second template strand comprises in 5′ to 3′ order: the second primer binding site, the nucleic acid sequence complementary to the target nucleic acid, and a nucleic acid sequence complementary to the first primer binding site.

37. The method of claim 36, wherein the first extension strand comprises in 5′ to 3′ order: the first primer, the target nucleic acid, and a nucleic acid sequence complementary to the second primer; and the second extension strand comprises in 5′ to 3′ order: the second primer, the nucleic acid sequence complementary to the target nucleic acid, and a nucleic acid sequence complementary to the first primer.

38. The method of claim 37, further comprising removing the nucleic acid sequence complementary to the first primer from the second extension strand, and removing the nucleic acid sequence complementary to the second primer from the first extension strand, to generate a double-stranded product comprising the target nucleic acid and the nucleic acid sequence complementary to the target nucleic acid.

39. The method of claim 38, wherein the nucleic acid sequence complementary to the first primer and the nucleic acid sequence complementary to the second primer are removed by digesting the first product strand and the second product strand with an enzyme comprising exonuclease activity.

40. The method of claim 39, wherein the enzyme comprising exonuclease activity is a DNA polymerase.

41. The method of claim 27, wherein the target nucleic acid has a length from about 160 to about 10,000 nucleobases.

42. A nucleic acid library comprising a plurality of double-stranded nucleic acids, each of the plurality of the double-stranded nucleic acids comprising:
a) a first strand comprising in 5′ to 3′ order: a first primer and a target nucleic acid, wherein the first primer comprises a first plurality of uracil nucleobases, wherein each uracil nucleobase of the first plurality of uracil nucleobases is distanced from another uracil nucleobase in the first primer by about 3 to about 8 non-uracil nucleobases, and wherein one uracil nucleobase of the first plurality of uracil nucleobases is a 3′ terminal uracil nucleobase bound to a preceding nucleobase in the first primer by a first nuclease resistant bond; and
b) a second strand comprising in 5′ to 3′ order: a second primer and a nucleic acid sequence complementary to the target nucleic acid, wherein the second primer comprises a second plurality of uracil nucleobases, wherein each uracil nucleobase of the second plurality of uracil nucleobases is distanced from another uracil nucleobase in the second primer by about 3 to about 8 non-uracil nucleobases, and wherein one uracil nucleobase of the second plurality of uracil nucleobases is a 3′ terminal uracil nucleobase bound to a preceding nucleobase in the second primer by a second nuclease resistant bond.

43. The nucleic acid library of claim 42, wherein each strand of the plurality of double-stranded nucleic acids has a length from about 160 to about 10,000 nucleobases.

44. The nucleic acid library of claim 43, wherein the first plurality of uracil nucleobases or the second plurality of uracil nucleobases comprises 3 or 4 uracil nucleobases.

45. The nucleic acid library of claim 42, wherein each uracil nucleobase of the first plurality of uracil nucleobases is distanced from another uracil nucleobase in the first primer by about 4 to about 7 non-uracil nucleobases.

46. The nucleic acid library of claim 42, wherein each uracil nucleobase of the second plurality of uracil nucleobases is distanced from another uracil nucleobase in the second primer by about 4 to about 7 non-uracil nucleobases.

47. The nucleic acid library of claim 42, wherein the nucleobase positioned immediately 5′ to the 3′ terminal uracil nucleobase of the first primer is a pyrimidine.

48. The nucleic acid library of claim 42, wherein the nucleobase positioned immediately 5′ to the 3′ terminal uracil nucleobase of the second primer is a pyrimidine.

49. The nucleic acid library of claim 47 or 48, wherein the pyrimidine is a cytosine nucleobase.

50. The nucleic acid library of claim 42, wherein the first nuclease resistant bond or the second nuclease resistant bond is a phosphorothioate bond.

51. The nucleic acid library of claim 42, wherein the length of the first primer or the second is from about 12 to about 50 nucleobases.

52. The nucleic acid library of claim 42, wherein the plurality of double-stranded nucleic acids is at least about 200 double-stranded nucleic acids.

53. The nucleic acid library of claim 42, wherein each first primer in the plurality of double-stranded nucleic acids comprises a first nucleic acid sequence that is the same.

54. The nucleic acid library of claim 42, wherein each second primer in the plurality of double-stranded nucleic acids comprises a second nucleic acid sequence that is the same.

55. A universal primer comprising: a non-naturally occurring sequence of nucleic acids comprising 3 or more uracil nucleobases; wherein one of the 3 or more uracil nucleobases is a 3′ terminal uracil nucleobase bound to a preceding nucleobase in the universal primer by a phosphorothioate bond; and wherein each of the 3 or more uracil nucleobases is distanced from a subsequent or a preceding uracil nucleobase in the universal primer by about 3 to about 8 non-uracil nucleobases.

56. The universal primer of claim 55, wherein the 3 or more uracil nucleobases is 3 or 4 uracil nucleobases.

57. The universal primer of claim 55, wherein each of the 3 or more uracil nucleobases is distanced from a subsequent or a preceding uracil nucleobase in the universal primer by about 4 to about 7 non-uracil nucleobases.

58. The universal primer of claim 55, wherein the universal primer has a length from about 12 to about 50 nucleobases.

59. The universal primer of claim 55, wherein the nucleobase preceding the 3′ terminal uracil nucleobase is a pyrimidine.

60. The universal primer of claim 59, wherein the pyrimidine is a cytosine nucleobase.

61. The universal primer of claim 55, wherein the melting temperature of the universal primer is between about 60° C. and about 66° C.

62. The universal primer of claim 55, wherein the GC content is between about 40% and about 60%.

63. A nucleic acid amplification reaction mixture comprising the universal primer of claim 55.
